(19) United States
(12) Patent Application Publication
(10) Pub. No.: US 2006/0014729 A1
(43) Pub. Date: Jan. 19, 2006

(54) Compounds and their preparation for the treatment of Alzheimer's disease by inhibiting beta-amyloid peptide production

(76) Inventors: Donald W. Landry, New York, NY (US); Tae-Wan Kim, East Brunswick, NJ (US); Shixian Deng, White Plains, NY (US)

Correspondence Address: BROWN RAYSMAN MILLSTEIN FELDER & STEINER LLP, 900 THIRD AVENUE, NEW YORK, NY, 10022, US

(21) Appl. No.: 10/963,214

(22) Filed: Oct. 12, 2004

Related U.S. Application Data
(60) Provisional application No. 60/588,433, filed on Jul. 16, 2004.

(51) Int. Cl. A61K 31/56; C07J 9/00

(52) U.S. Cl. 514/182; 552/540

(57) ABSTRACT

The present invention provides novel dammarane compounds, compositions (e.g., pharmaceutical compositions) comprising the dammarane compounds, and methods for the synthesis of these dammarane compounds. Additionally, the present invention provides methods for inhibiting beta-amyloid peptide production and methods for treating or preventing a pathological condition, particularly, neurodegeneration diseases (e.g., Alzheimer's disease), using these dammarane compounds.

### CROSS REFERENCE TO RELATED APPLICATIONS

The present application claims the benefit of U.S. application Ser. No. ______ (not yet assigned), filed Oct. 7, 2004, which claims the benefit of U.S. Application Ser. No. 60/588,433, filed Jul. 16, 2004, which is incorporated herein by reference thereto.

### STATEMENT OF GOVERNMENT INTEREST

This invention was made in part with government support under NIH Grant No. ROI N543467. As such, the United States government may have certain rights in this invention.

### FIELD OF THE INVENTION

The present invention provides novel compounds, compositions (e.g., pharmaceutical compositions) comprising the dammarane compounds, and methods for the synthesis of these compounds. Additionally, the present invention provides methods for inhibiting beta-amyloid peptide production and methods for treating or preventing a pathological condition, particularly, neurodegenerative diseases (e.g., Alzheimer's disease), using these dammarane compounds.

### BACKGROUND OF THE INVENTION

The amyloid-β peptide (Aβ), a proteolytic fragment of 39-43 amino acids derived from the integral membrane glycoprotein amyloid-β precursor protein (APP) (Kang et al., Nature, 325, pp. 733(1987); U.S. Pat. No. 6,262,302) participates in the pathogenesis of a variety of illness. Examples include progressive neurodegenerative diseases, e.g., Alzheimer's disease (“AD”) or related Aβ-mediated dementia, and certain cancers, such as breast and endometrial cancers (He et al., J. Biol. Chem., 274(21), 15014(1999)).

Alzheimer's disease (AD) is a progressive neurodegenerative disease of the brain resulting in diminished cognitive abilities, dementia, and ultimately death. A strong link has been established between the development of AD and the extracellular accumulation of Aβ in the brain. Aggregated Aβ appears to be toxic to neuronal cells in culture and has been reported to cause apoptotic cell death in vitro through the generation of nitric oxide and other free radicals. Aβ has also been reported to form plaques inside and outside nerve cells [Wilson et al., Journal of Neuropathology And Experimental Neurology, vol. 58, 787(1999)]. These plaques are strongly correlated with dementia and it is proposed that they disrupt the function of calcium channels.

Ginseng is the common name given to the dried roots of plants of the genus Panax which has been used extensively in Asia for thousands of years as a general health tonic and medicine for treating an array of diseases (Cho, et al. (1995) Pharmacological action of Korean ginseng. In the Society for Korean Ginseng (eds.): Understanding Korean Ginseng, Seoul: Hanlim Publishers, pp 35-54; Shibata S. (2001) Chemistry and cancer preventing activities of ginseng saponins and some related triterpenoid compounds. J Korean Med Sci. 16 Suppl:S28-37; Attele, et al. (1999); Ginseng pharmacology: multiple constituents and multiple actions. Biochem Pharmacol. 58:1685-1693; Coleman, et al. (2003). The effects of Panax ginseng on quality of life. J. Clin. Pharm. Ther. 28, 5-15; Coon and Ernst (2002). Panax ginseng: a systematic review of adverse effects and drug interactions. Drug Saf. 25:323-44). The Panax genus contains about six species native to eastern Asia and two species native to eastern North America. Panax ginseng (Asian ginseng) and Panax quinquefolius L. (North American ginseng) are the two species most commonly used in nutraceutical and pharmaceutical compositions. The roots and their extracts contain a variety of substances including saponins.

Ginseng has been well known to have specific pharmacological effects including improvement of liver function and immune enhancement, as well as anti-arteriosclerotic, anti-thrombotic, anti-stress, anti-diabetic, anti-hypertensive and anti-tumor effects. Among several classes of compounds isolated from the ginseng root, ginseng saponins are known to be the chemical constituents that contribute to its diverse pharmacological effects. (Kwon, et al. (2001) J. Chromatogr. A. 921; 335; Park, et al. (2002) Chem. Pharm. Bul. 50, 538; Park, et al. (2002) Arch. Pharm. Res. 25, 428, Kim, et al. (2000) J. Nat. Prod. 63:1702). The ginseng saponins (also known as dammaranes) are triterpene glycosides. To date, at least 31 dammaranes have been isolated from white and red ginseng. The dammaranes can be divided into three groups depending on their aglycons: protopanaxadiol-type dammaranes (e.g., Rb1, Rb2, Rc, Rd, (20R)Rg3, (20S)Rg3, Rh2), protopanaxatriol-type dammaranes (e.g., Re, Rf, Rg1, Rg2, Rh1), and oleanolic acid-type dammaranes (e.g., Ro). Both protopanaxadiol-type and protopanaxatriol-type dammaranes have a triterpene backbone structure, known as dammarane (Attele, et al. (1999) Ginseng pharmacology: multiple constituents and multiple actions. Biochem. Pharmacol. 58:1685-1693). Rk1, Rg5 (20R)Rg3 and (20S)Rg3 are dammaranes that are almost uniquely present in heat-processed ginseng, but are not found to exist as trace elements in unprocessed ginseng (Kwon, et al. (2001) Liquid chromatographic determination of less polar dammaranes in processed ginseng. J. Chromatogr. A. 921;335-339; Park, et al. (2002); Cytotoxic dammarane glycosides from processed ginseng. Chem. Pharm. Bul. 50, 538-540 Park, et al. (2002); Three new dammarane glycosides from heat-processed ginseng. Arch. Pharm. Res. 25, 428-432; Kim, et al. (2000); Steaming of ginseng at high temperature enhances biological activity. J. Nat. Prod. 63:1702-1702).

Processing of ginseng with steam at high temperature further enhances the content of these unique dammaranes Rk1, Rg5, (20R)Rg3 and (20S)Rg3, which appear to possess novel pharmacological activities.

U.S. Pat. No. 5,776,460 (“the '460 patent”) discloses a processed ginseng product having enhanced pharmacological effects. This ginseng product, commercially known as “sun ginseng,” contains increased levels of effective pharmacological components due to heat-treating of the ginseng at a high temperature for a particular period of time. As specifically disclosed in the '460 patent, heat treatment of ginseng may be performed at a temperature of 120° to 180° C. for 0.5 to 20 hours, and is preferably performed at a temperature of 120° to 140° C. for 2 to 5 hours. The heating time varies depending on the heating temperature such that lower heating temperatures require longer heating times while higher heating temperatures require comparatively shorter heating times. The '460 patent also discloses that the processed ginseng product has pharmacological properties specifically including anti-oxidant activity and vasodilation activity.

Recently, the inventors of the present application, Tae-Wan Kim, et al., demonstrated that the unique components of the heat-processed ginseng product disclosed in the '460 patent significantly lower the production of Aβ42 in cells (patent application pending). Specifically, the inventors discovered that at least three dammaranes Rk1, (20S)Rg3, and Rg5, unique components of the heat-processed ginseng known as “Sun Ginseng,” as well as Rgk351, which is a mixture of (20R)Rg3, (20S)Rg3, Rg5, and Rk1, lower the production of Aβ42 in mammalian cells. Rgk351 and Rk1 were most effective in reducing Aβ42 levels. Further, Rk1 was also shown to inhibit the Aβ42 production in a cell-free assay using a partially purified γ-secretase complex, suggesting that Rk1 modulates either specificity and/or activity of the γ-secretase enzyme. In addition, Tae-Wan Kim, et al., found that certain dammaranes that harbor no Aβ42-reducing activity in vitro, are effective in reducing Aβ42 in vivo. For example, some of the 20(S)-protopanaxatriol (PPT) group dammaranes, such as Rg1, can be converted into PPT after oral ingestion. Thus, while Rg1 generally has no amyloid-reducing activity in vitro, Rg1 may be converted into an active amyloid-reducing compound PPT in vivo.

However, due to the extremely low yield of these rare components from ginseng, the difficulty of cultivating ginseng and the problematic synthesis of the dammarane core structure, the development of these components to pharmaceutical agents is severely impeded. To find structurally simpler analogs or inexpensive sources for starting materials is crucial for drug development. Dammaranes are structurally similar to chemical constituents of other plants. These plants share the triterpene backbone structure, known as dammarane, although they differ in number, position and configuration of hydroxyl groups on the dammarane ring. For example, the major constituent of dammar resin, 20-hydroxy-(20S)-Dammar-24-en-3-one, from trees of the Dipertocarpaoeae family and of the genus agathis (such as Amboynas pine), differ from 20(S)-protopanaxadiol ginseng only at 12-hydroxyl group. Betulafolienetriol [dammar-24-ene-(3α, 12β)-3,20(S)-triol], isolated from birch leaves differ from 20(S)-protopanaxatriol ginseng, only in the configuration at C-3.

Birch (Betula platyphylla) has been extensively used as a medicinal plant in many areas of far-east Asia as well as Northern Europe. Traditionally, birch has been used for many ailments ranging from headache to fever, cramps, gout, wounds and skin ailments (David Hoffmann (199) The New Holistic Herbal). The components extracted from birch leave show various biological activities such as anti-cancer and, hemolytic activities (Fuchino, H. Chemical & Pharmaceutical Bulletin (1998), 46(1), 169; Chemical & Pharmaceutical Bulletin (1998), 46(1), 166; Hilpisch, U. Planta Medica (1997), 63(4), 347; Pokhilo, N. D., Khimiya Prirodnykh Soedinenii (1994), (5), 681). More than a dozen patents disclose the process of isolation of natural products from birch bark and their biological activity (WO 2001010885; Russ. (1999), RU 2131882; Jpn. Kokai Tokkyo Koho JP 2003192694). The components extracted from birch bark show antitumor, antiviral (HIV-1), anti-inflammatory and antiprotozoal properties (U.S. Pat. No. 5,750,578). Among them, Betulin, one of the major components, possesses antiviral activity and is used to prepare ablobetulin and derivatives which possess useful pharmacological properties. The components from birch leaves have been previously transformed into dammarane Rh2 (Atopkina, Carbohydrate Research (1997), 303(4), 449-451). WO9603419 disclosed that Dammar resin was used for preparation of its 17α-isomer with immunosuppressant and anti-inflammatory activities.

Considering the structural similarity and their abundance, the inventors decided to investigate natural products from other plants as sources for active compounds and/or starting materials to synthesize the active compounds that lower Aβ42 production in mammalian cells. The inventors disclose herein compounds having the general structure: 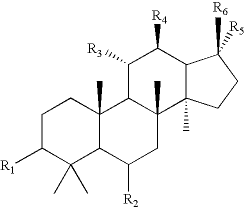
(general structure I) that are synthesized or extracted from readily available and inexpensive sources such as dammar resin and birch tree. Further, the present invention provides methods to chemically synthesize the compounds of general structure I including dammaranes Rg3, Rg5, and Rk1. Accordingly, the present invention provides novel compounds and methods for synthesizing these compounds from extracts from plants such as birch. Additionally, this invention provides methods for using these compounds for inhibiting beta-amyloid peptide production, as well as for treating or preventing neurodegeneration associated disorders (e.g., Alzheimer's disease).

### SUMMARY OF THE INVENTION

The present invention provides compounds, compositions and methods for preventing and treating neurodegenerative diseases, such as Alzheimer's disease, by inhibiting beta-amyloid peptide production. The present invention also provides methods of synthesis of the compounds and compositions.

In one aspect, the present invention provides a compound having the general formula: 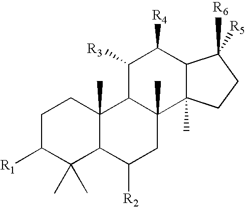
where R₁ is selected from the group consisting of α-OH, β-OH, α-O—X, β—O—X, α-R₇COO—, β-R₇COO—, α-R₇PO₃—, β—R₇PO₃—, α-NR₈R₉, β-NR₈R₉, ═O(oxo), ═NOH, ═NC(O)NHNH₂, and CH₂—X; where X is an alkyl or a carbohydrate containing one or more sugars or acylated derivatives thereof; R₇ is H, OH, an amino group, an alkenyl, aryl, or alkyl; R₈ is H, alkyl, aryl, acyl, or SO₂NHR₁₀; R₉ is H, alkyl, aryl, acyl, or SO₂NHR₁₀; R₁₀ is NH₂, OH, alkyl, aryl, or cycloalkyl; R₂ is selected from the group consisting of H, OH, OAc, and O—X, where X is a carbohydrate containing one or more sugars or acylated derivatives thereof, Ac═CH₃CO or acyl; R₃ is selected from the group consisting of H, OH, O—X and OAc, where X is an alkyl or a carbohydrate containing one or more sugars or acylated derivatives thereof; R₄ is selected from the group consisting of H, OH, O—X and OAc, where X is an alkyl or a carbohydrate containing one or more sugars or acylated derivatives thereof; R₅ is H or OH; and R₆ is alkenyl, aryl, or alkyl. R₇ may further contain oxygen, nitrogen, or phosphorus and R₆ may further contain a functional group selected from the group consisting of hydroxyl, ether, ketone, oxime, hydrazone, imine, and Schiff base. In one embodiment, the sugar group is selected from the group consisting of Glc, Ara(pyr), Ara(fur), Rha, and Xyl, and acylated derivatives thereof. In another embodiment, R₆ is selected from the group consisting of: 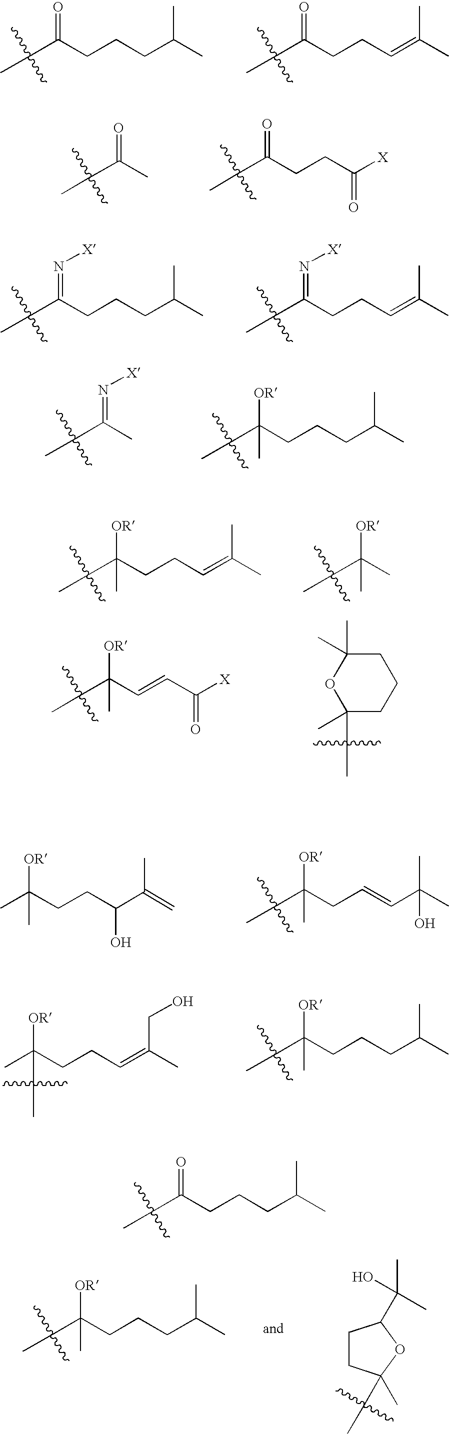
where the configuration of any stereo center is R or S; X is OR or NR, X′ is alkyl, OR, or NR; and where R is alkyl or aryl; and R′ is H, alkyl, or acyl.

In another embodiment, the present invention provides a composition, particularly, a pharmaceutical composition, comprising a compound having the general formula: 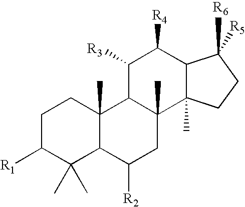
where R₁ is selected from the group consisting of α-OH, β-OH, α-O—X, β-O—X, α-R₇COO—, β-R₇COO—, α-R₇PO₃—, α-R₇PO₃—, α-NR₈R₉, β-NR₈R₉, ═O(oxo), ═NOH, ═NC(O)NHNH₂, and CH₂—X; where X is an alkyl or a carbohydrate containing one or more sugars or acylated derivatives thereof; R₇ is H, OH, an amino group, an alkenyl, aryl, or alkyl; R₈ is H, alkyl, aryl, acyl, or SO₂NHR₁₀; R₉ is H, alkyl, aryl, acyl, or SO₂NHR₁₀; R₁₀ is NH₂, OH, alkyl, aryl, or cycloalkyl; R₂ is selected from the group consisting of H, OH, OAc, and O—X, where X is a carbohydrate containing one or more sugars or acylated derivatives thereof, Ac═CH₃CO or acyl; R₃ is selected from the group consisting of H, OH, O—X and OAc, where X is an alkyl or a carbohydrate containing one or more sugars or acylated derivatives thereof;

R₄ is selected from the group consisting of H, OH, O—X and OAc, where X is an alkyl or a carbohydrate containing one or more sugars or acylated derivatives thereof; R5 is H or OH; and R6 is alkenyl, aryl, or alkyl. R₇ may further contain oxygen, nitrogen, or phosphorus and R₆ may further contain a functional group selected from the group consisting of hydroxyl, ether, ketone, oxime, hydrazone, imine, and Schiff base.

In a preferred embodiment of the present invention the compound or composition is selected from the group consisting of: 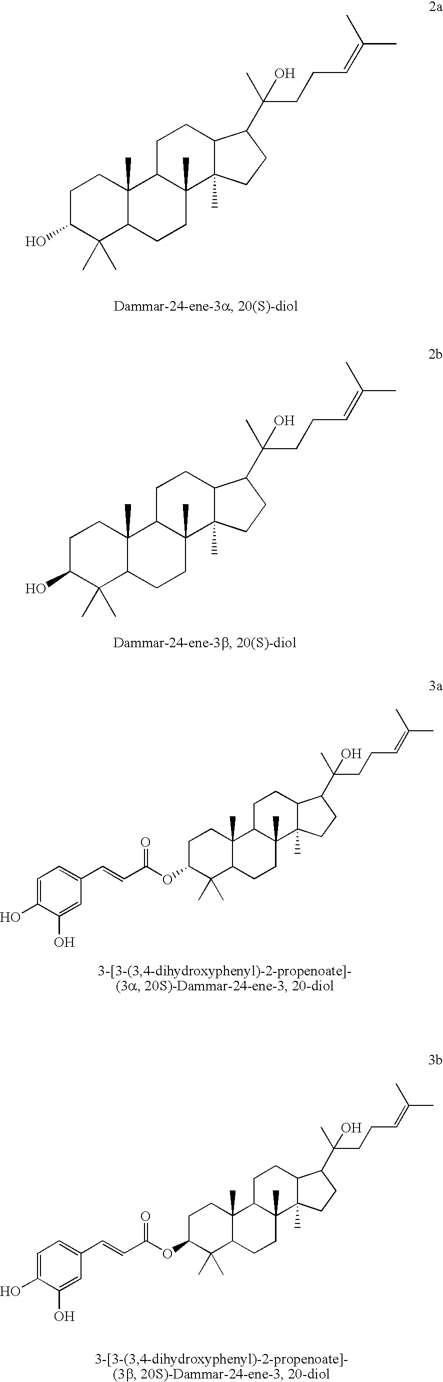 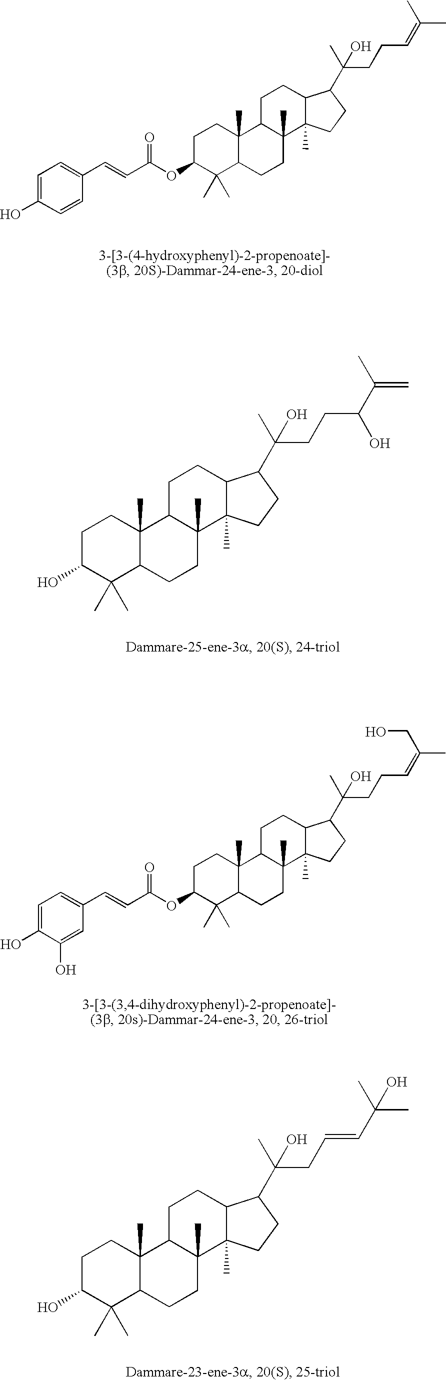 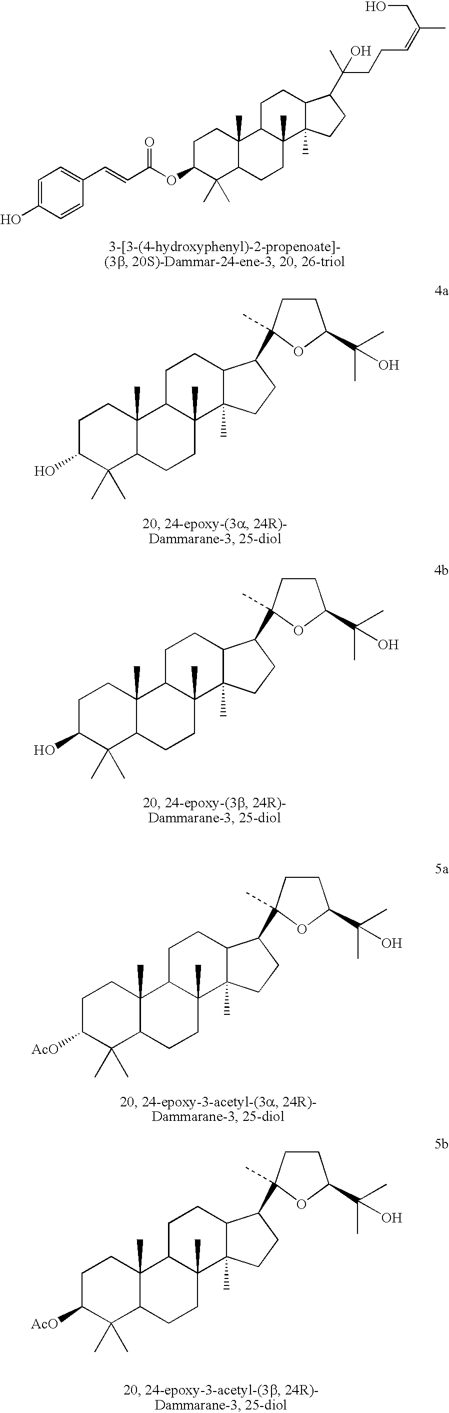 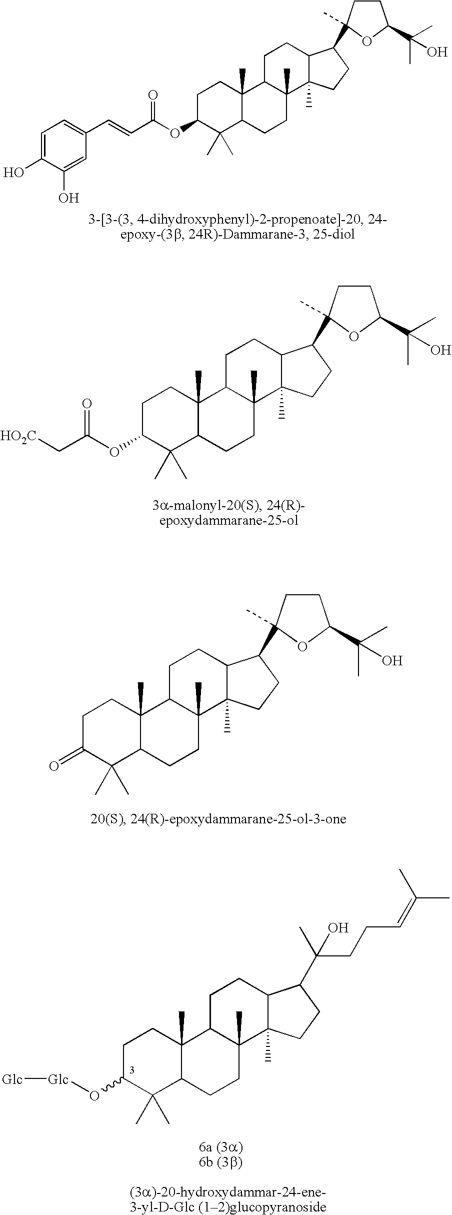 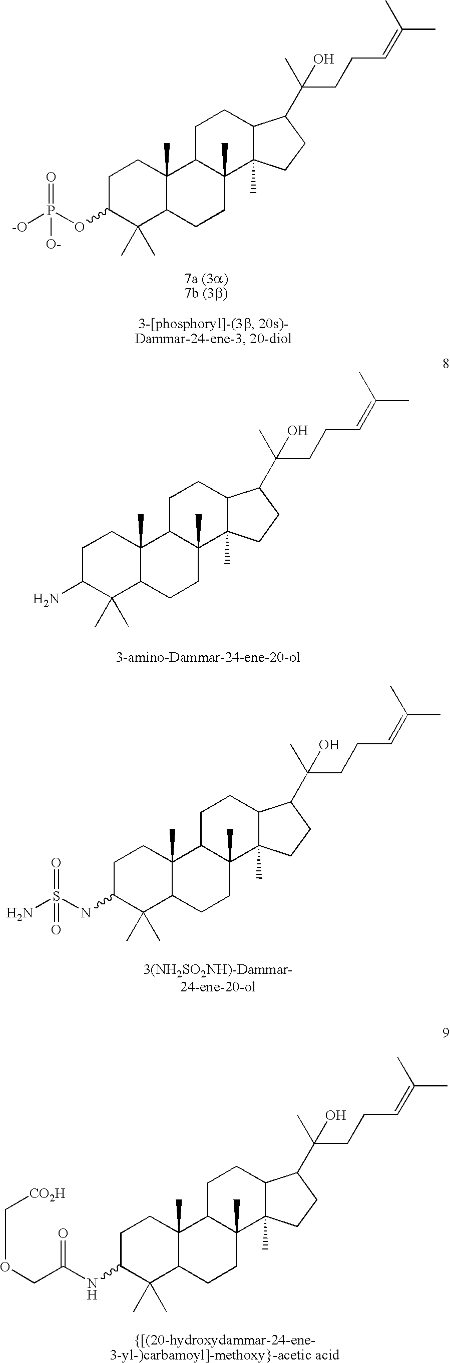 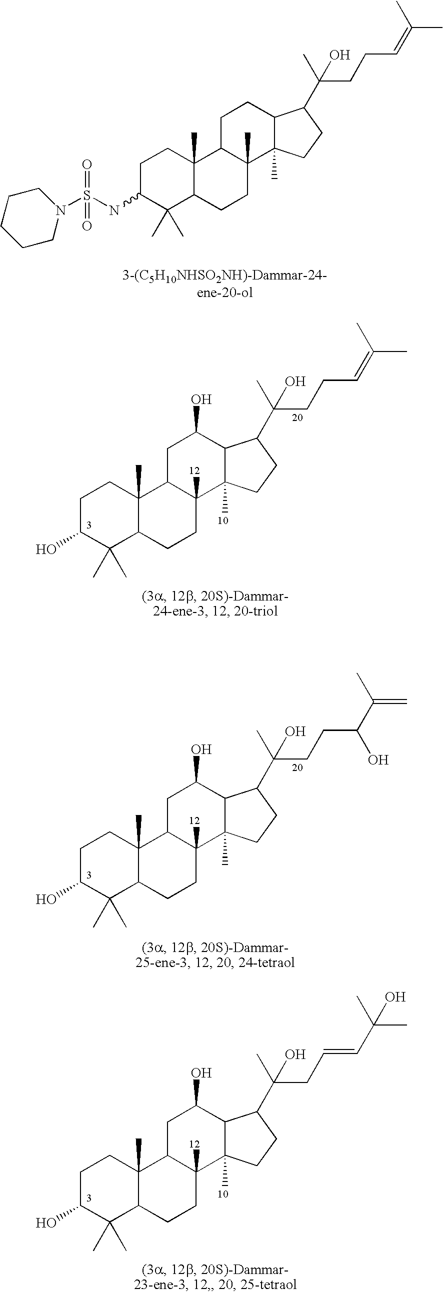 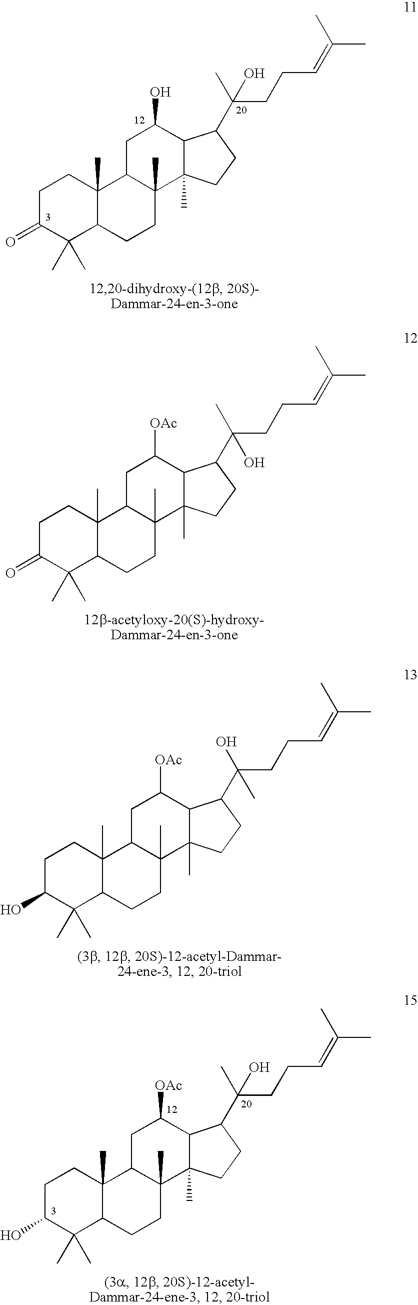 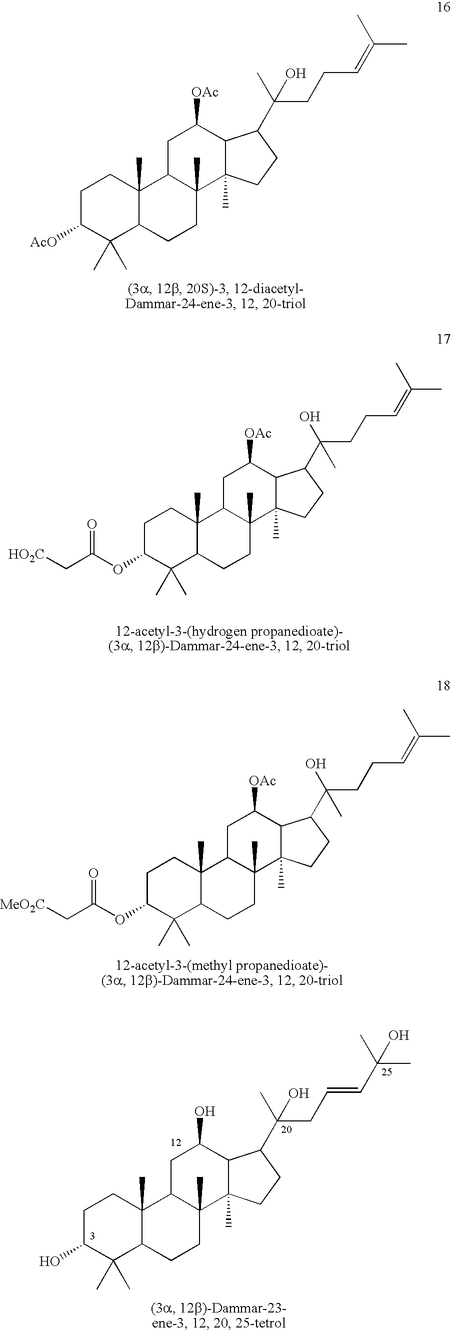 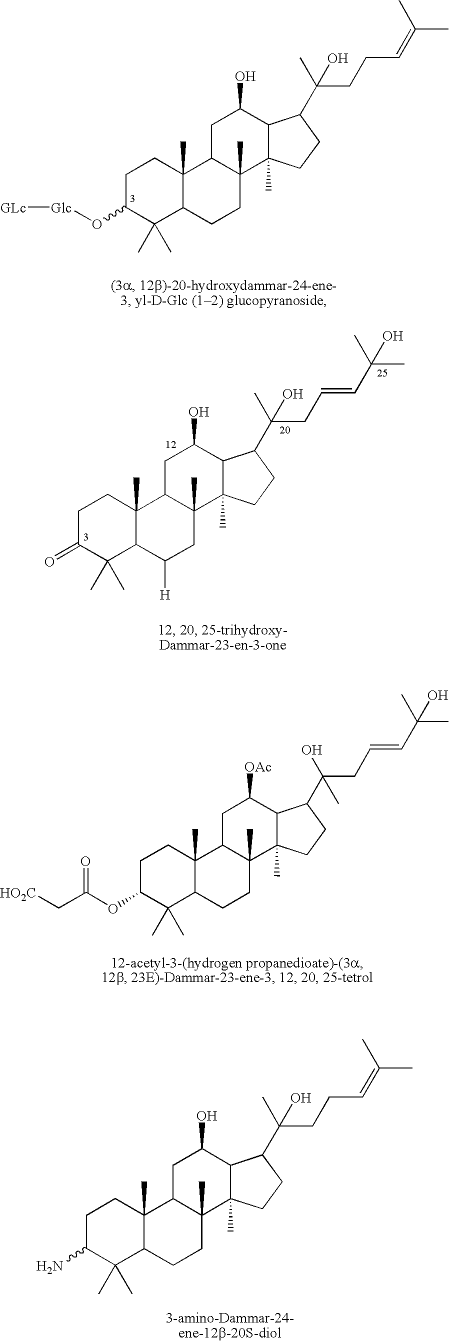 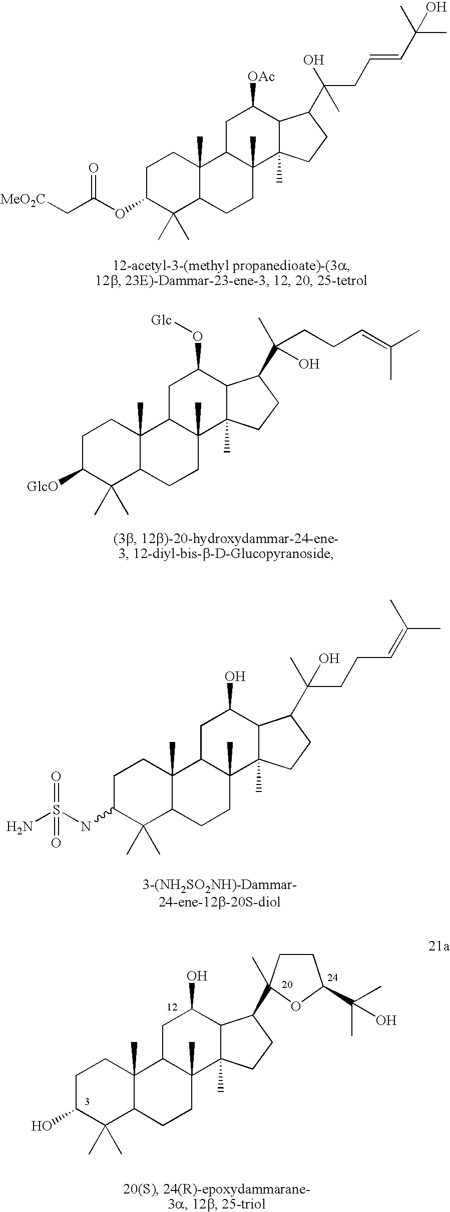 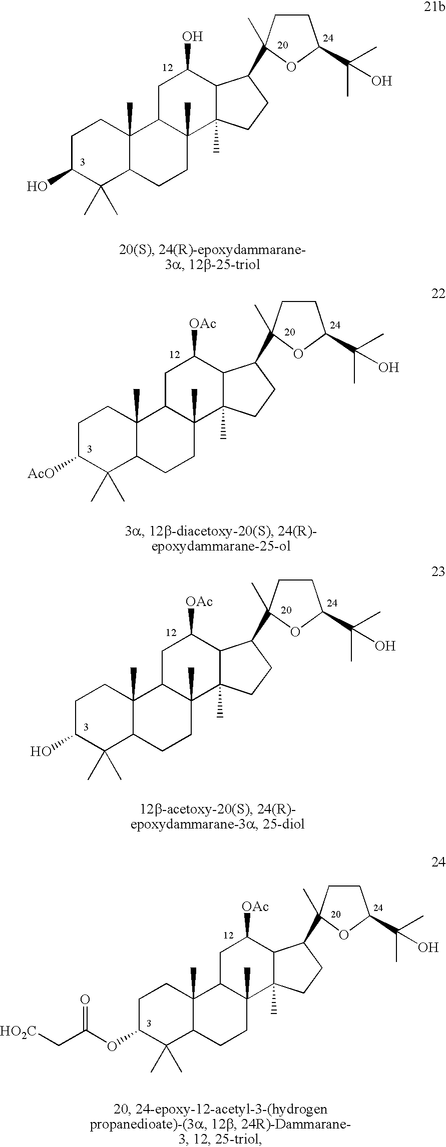 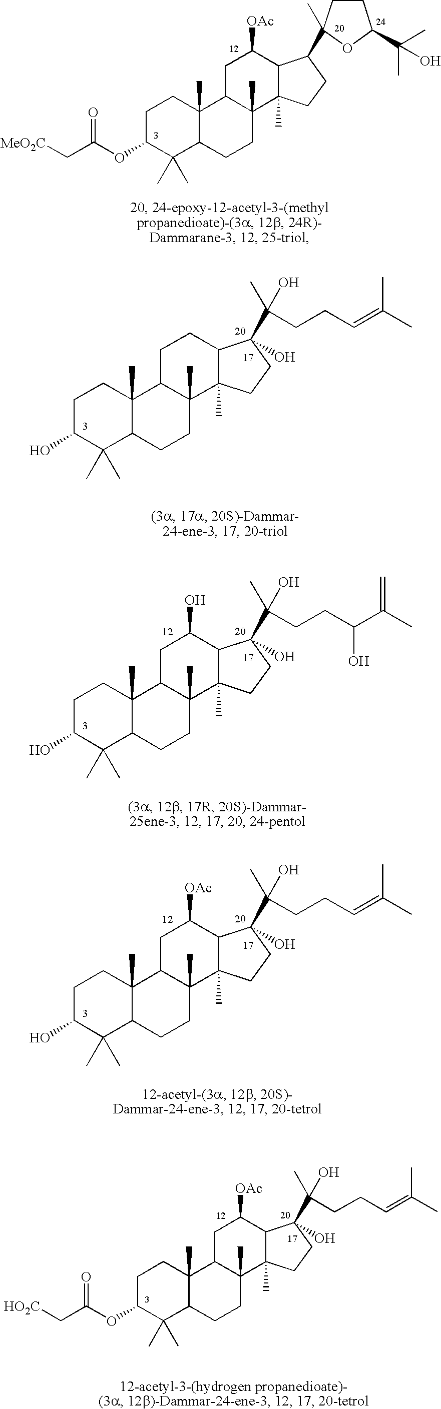 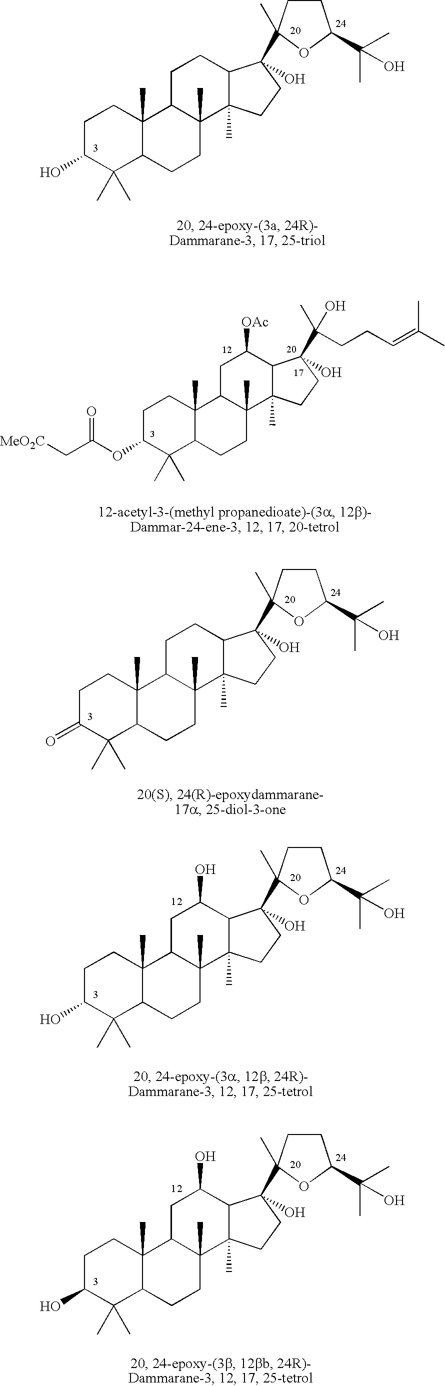 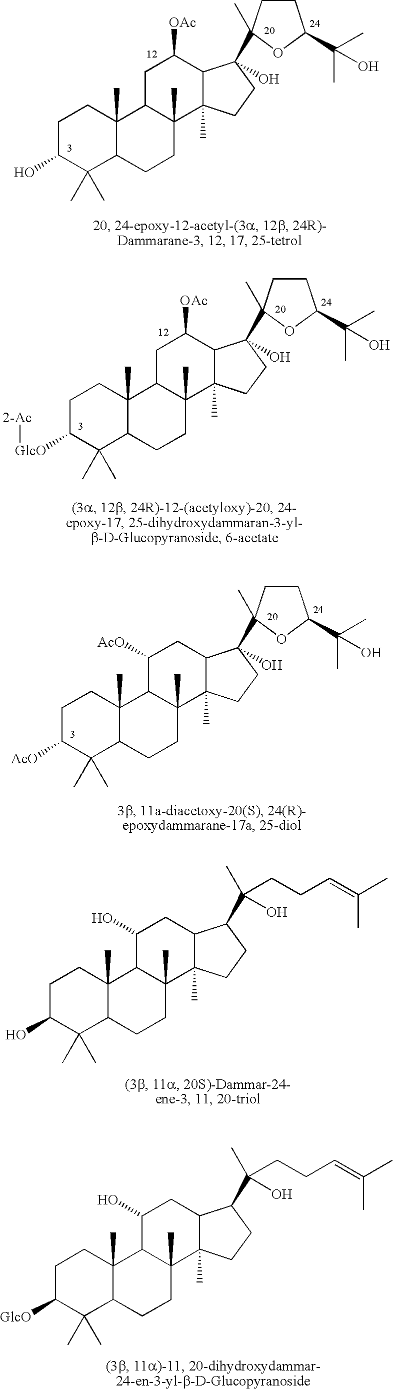 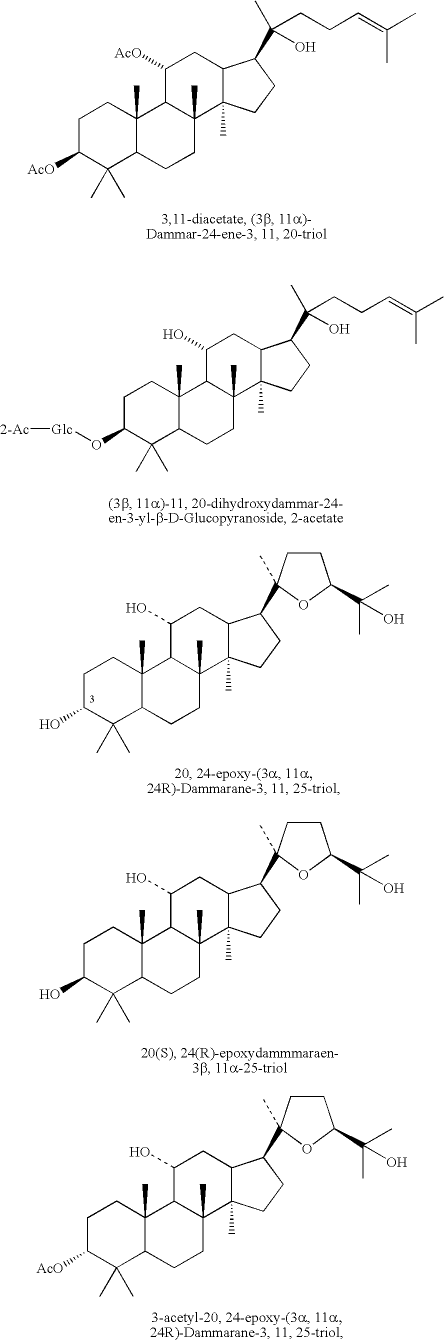 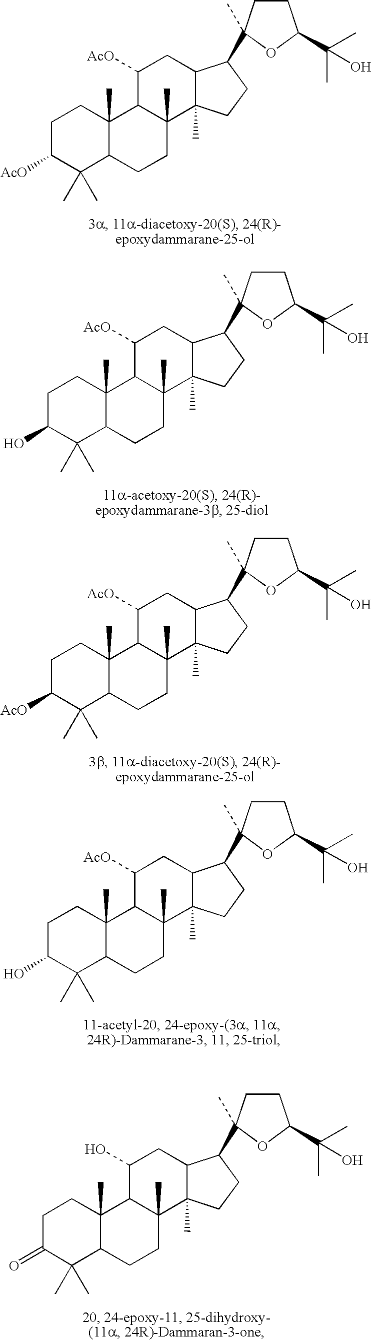 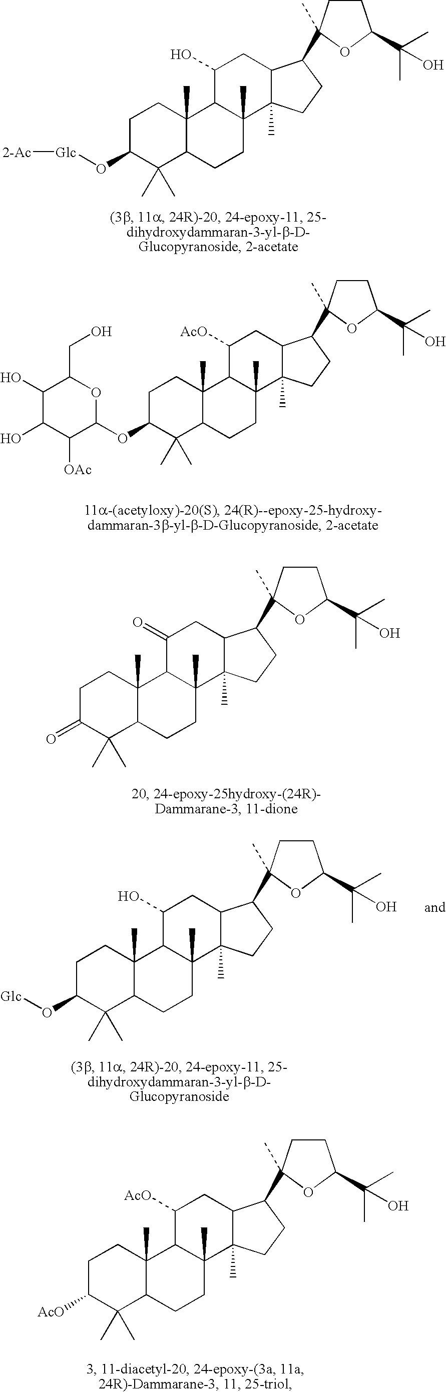
The pharmaceutical composition of the present invention may comprise a pharmaceutically acceptable carrier.

The present invention also provides a method for the synthesis of a compound having formula: 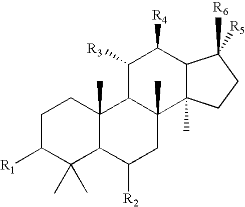
which comprises the steps of:
(a) treating a compound having formula: 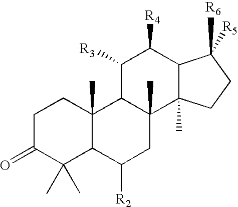
   with a reducing agent, followed by an appropriate oxidizing agent to form a compound having formula: 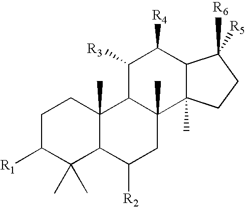
where R₁ is selected from the group consisting of oα-OH, β-OH, α-O—X, β-O—X, α-R₇COO—, β-R₇COO—, α-R₇PO₃—, β-R₇PO₃—, α-NR₈R₉, β-NR₈R₉, ═O(oxo), ═NOH, ═NC(O)NHNH₂, and CH₂—X; where X is an alkyl or a carbohydrate containing one or more sugars or acylated derivatives thereof; R₇ is H, OH, an amino group, an alkenyl, aryl, or alkyl; R₈ is H, alkyl, aryl, acyl, or SO₂NHR₁₀; R₉ is H, alkyl, aryl, acyl, or SO₂NHR₁₀; R₁₀ is NH₂, OH, alkyl, aryl, or cycloalkyl; R₂ is selected from the group consisting of H, OH, OAc, and O—X, where X is a carbohydrate containing one or more sugars or acylated derivatives thereof, Ac═CH₃CO or acyl; R₃ is selected from the group consisting of H, OH, O—X and OAc, where X is an alkyl or a carbohydrate containing one or more sugars or acylated derivatives thereof; R₄ is selected from the group consisting of H, OH, O—X and OAc, where X is an alkyl or a carbohydrate containing one or more sugars or acylated derivatives thereof; R5 is H or OH; and R6 is alkenyl, aryl, or alkyl.

The present invention further provides a method for the synthesis of a compound having formula: 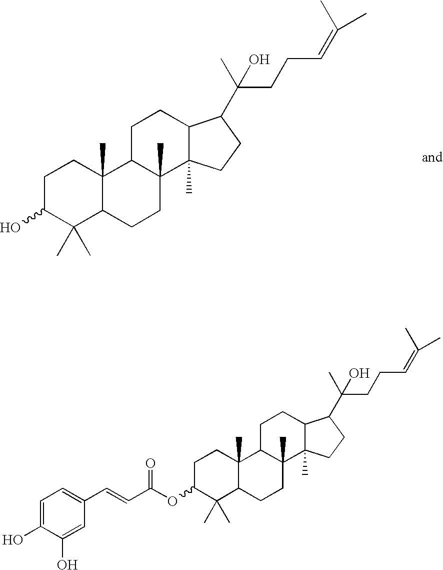
said method comprising the steps of:
(a) treating a compound having formula: 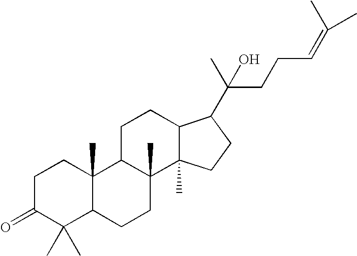
   with a reducing agent, to form a compound having formula: 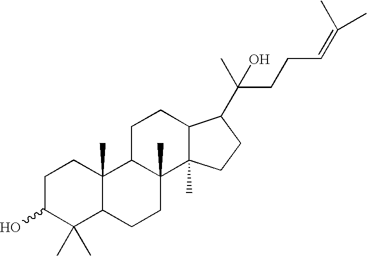
(b) treating the compound formed in step (a) with an esterificating agent followed by a hydrolyzing agent, to form a compound having formula: 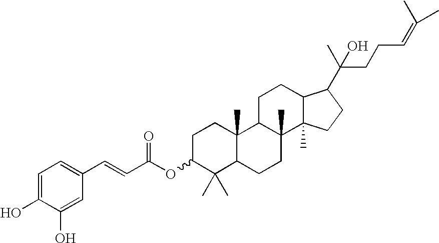
   In one embodiment, the reducing agent is NaBH₄ In another embodiment, the esterificating agent is di-O-acetylcaffeoyl chloride and the hydrolyzing agent is NaHCO₃.

Additionally, the invention provides a method for the synthesis of a compound having formula: 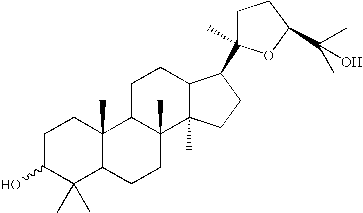
the method comprising the steps of:
(a) treating a compound having formula: 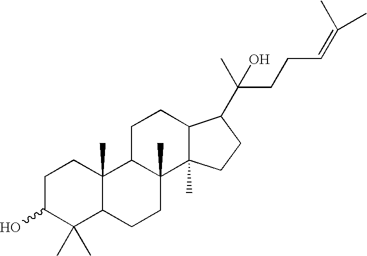
   with an oxidizing agent, to form a compound having formula: 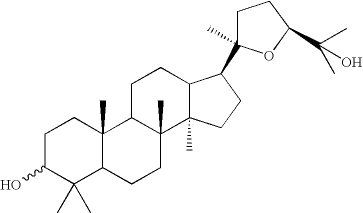
(b) treating the compound formed in step (a) with an esterificating agent to form a compound having formula: 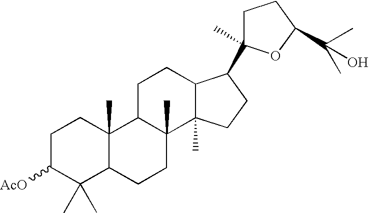
   In one embodiment, the oxidizing agent is MCPBA. In another embodiment, the esterificating agent is Ac₂O.

In still another aspect, the present invention provides a method for the synthesis of a compound having formula: 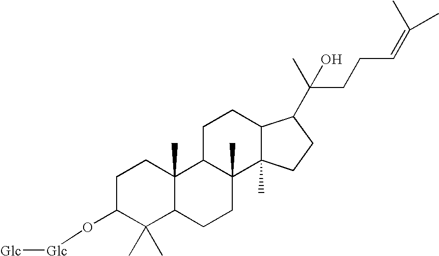
the method comprising the steps of:
(a) treating a compound having formula: 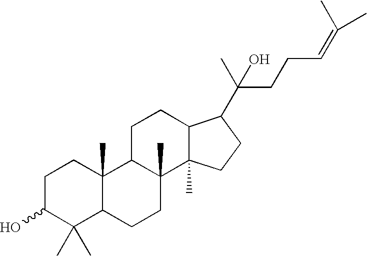
   with an appropriate bromonated sugar and an oxidizing agent, followed by deprotection of the acetyl groups to form a compound having formula: 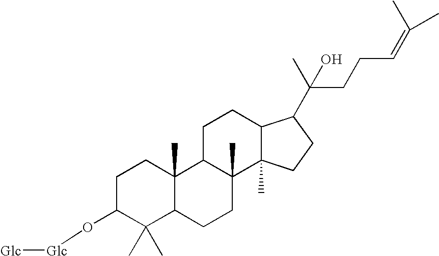
   In one embodiment, the oxidizing agent is Ag2O. In yet another embodiment, the compound is deprotected using NaOMe.

The present invention further provides a method for the synthesis of a compound having formula: 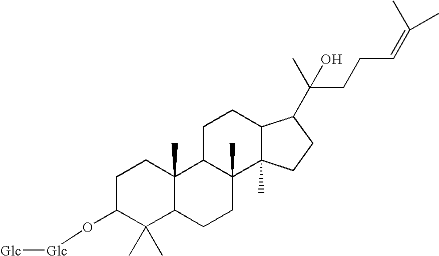
the method comprising the steps of:
(a) treating a compound having formula: 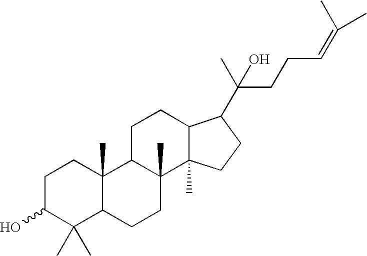
   with an appropriate bromonated sugar and an oxidizing agent, followed by deprotection of the acetyl groups to form a compound having formula: 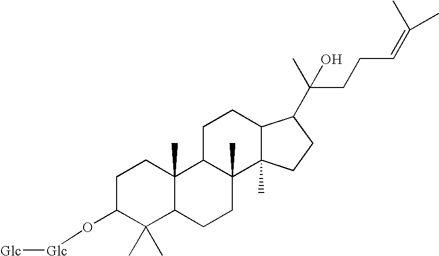
   In one embodiment, the oxidizing agent is Ag2O. In another embodiment, the compound is deprotected using NaOMe.

The present invention also provides a method for the synthesis of a compound having the formula: 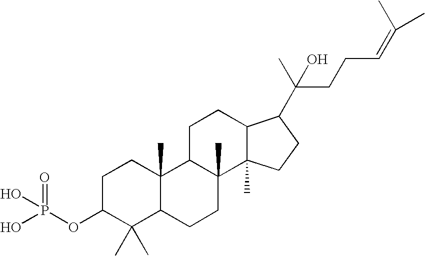
The method comprising the steps of:
(a) treating a compound having formula: 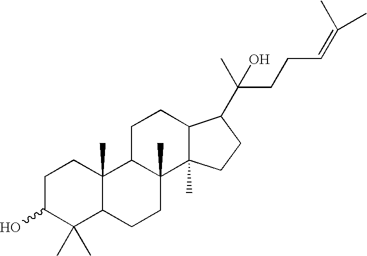
   with a dibenzylphosphoric agent and a base to form a compound having formula: 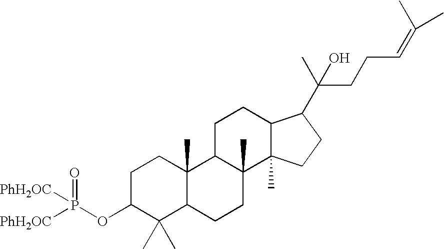
(b) treating the compound formed in step (a) with a hydrogenating agent to form a compound having formula: 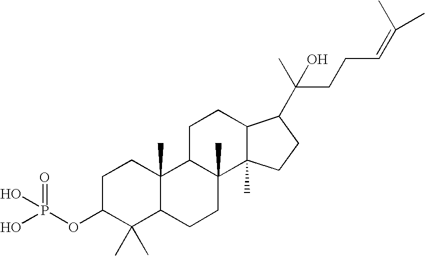
   In one embodiment, the dibenzylphosphoric agent is (PhCH₂O)₂POCl. In another embodiment, the hydrogenating agent is H₂/Pd—C.

The present invention further provides a method for the synthesis of a compound having the formula: 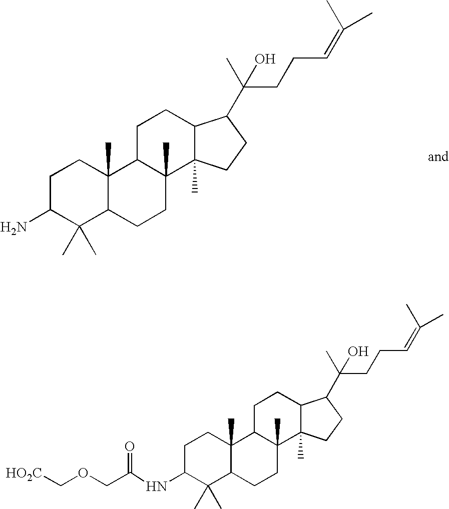
the method comprising the steps of:
(a) treating a compound having formula: 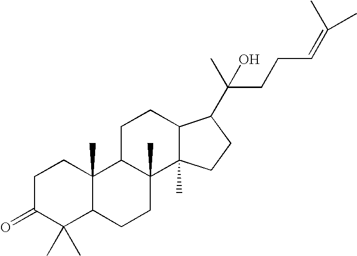
   with a reducing agent to form a compound having formula: 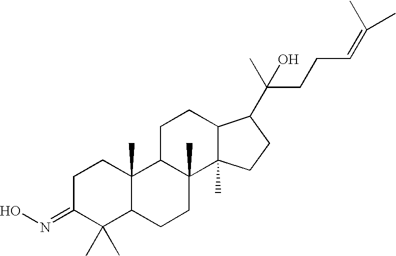
(b) treating the compound formed in step (a) with an additional reducing agent to form a compound having formula: 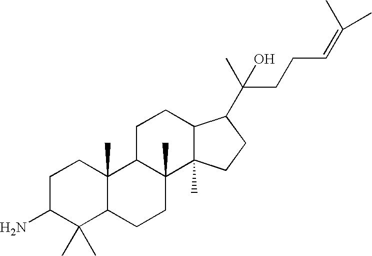
(c) treating the compound formed in step (b) with diglylcolic anhydride in the presence of a base to form a compound having formula: 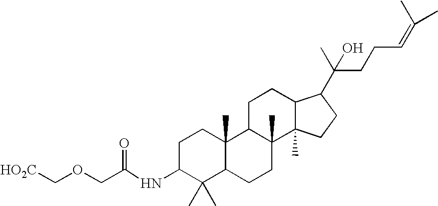
   In one embodiment, the reducing agent of step (a) is NH₂OH. In another embodiment, the reducing agent of step (b) is Na.

The present invention also provides a method for the synthesis of a compound having the formula: 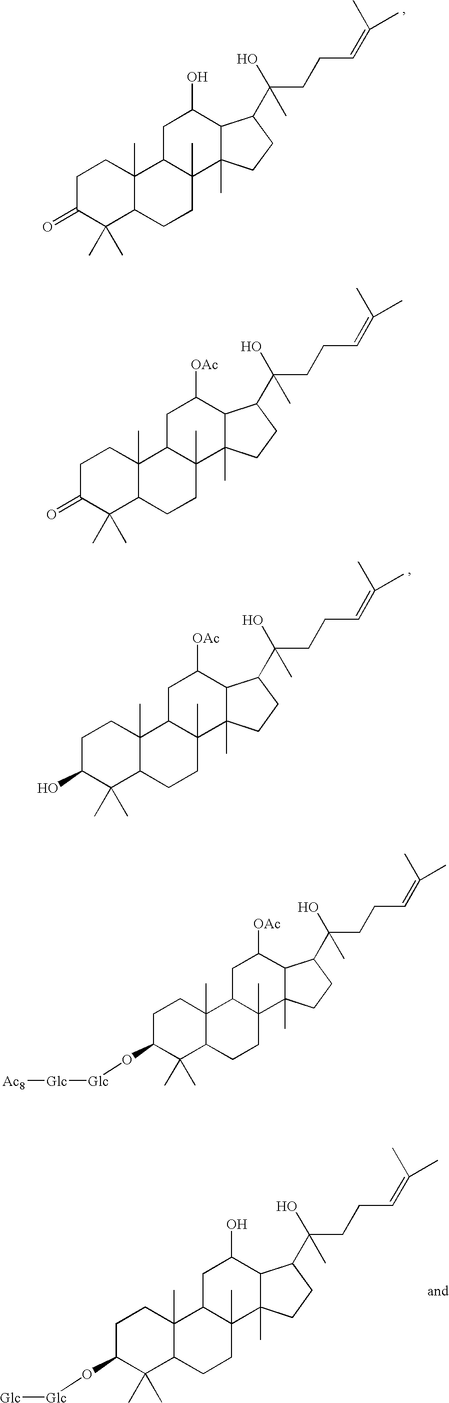 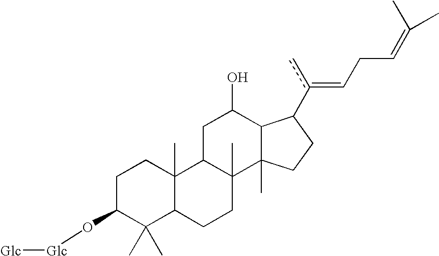
the method comprising the steps of:
(a) treating a compound having formula: 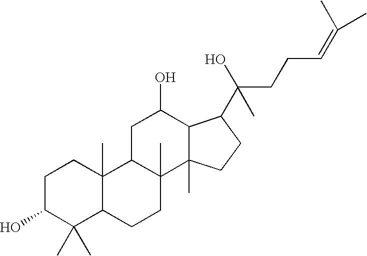
   with an oxidizing agent to form a compound having formula: 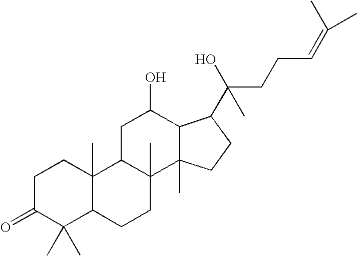
(b) treating the compound formed in step (a) with an acetylating agent to form a compound having formula: 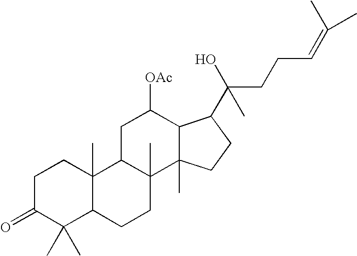
(c) treating the compound formed in step (b) with a reducing agent to form a compound having formula: 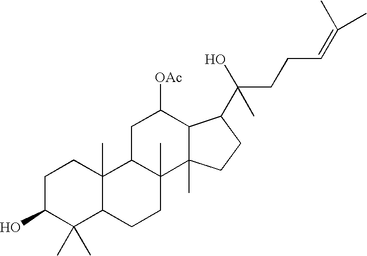
(d) treating the compound formed in step (c) with Ac₈-Glc-Glc-Br in the presence of Ag₂O to form a compound having formula: 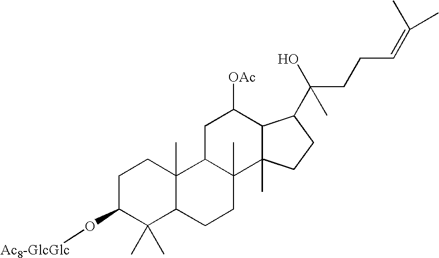
(e) treating the compound formed in step (d) with a deacetylating agent to form a compound having formula: 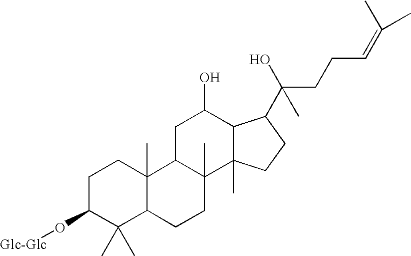
(f) treating the compound formed in step (e) with a dehydrating agent to form a compound having formula: 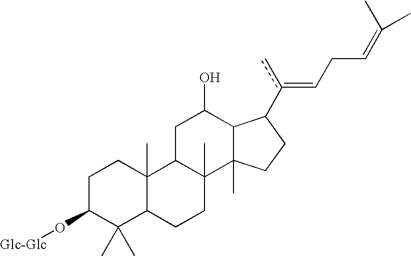
   In one embodiment, the starting compound is obtained from a plant, such as, for example, birch. In another embodiment, the oxidizing agent is CrO₃ in pyridine. In a further embodiment, the acetylating agent is Ac₂O in pyridine. In another embodiment, the reducing agent is NaBH₄. In another embodiment, the dehydrating agent is mesyl chloride and triethylamine. In yet another embodiment, the deacetylating agent is NaOMe.

The present invention also provides a method for the synthesis of a compound having the formula: 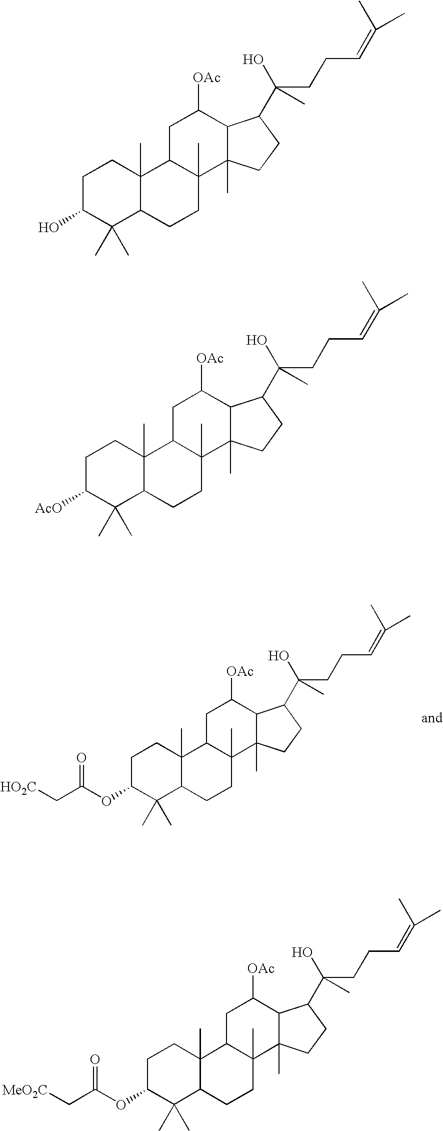
the method comprising the steps of:
(a) treating a compound having formula: 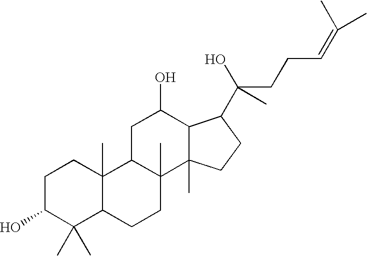
   with Ac₂O to form a compound having formula: 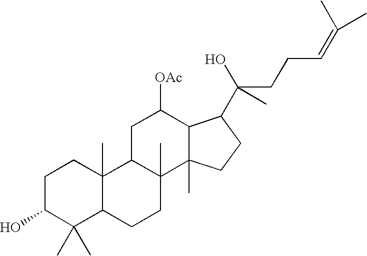
(b) treating the compound formed in step (a) with Ac₂O to form a compound having formula: 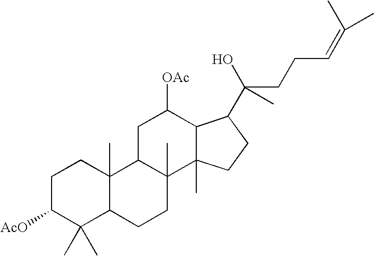
(c) treating the compound formed in step (a) with CH₂(COCl)₂ to form a compound having formula: 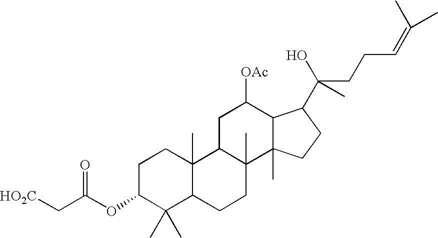
(d) treating the compound formed in step (c) with CH₂N₂ to form a compound having formula: 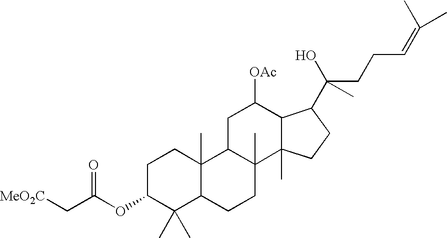
   In one embodiment, the starting compound is obtained from a plant, such as, for example, birch.

The present invention additionally provides a method for the synthesis of a compound having the formula: 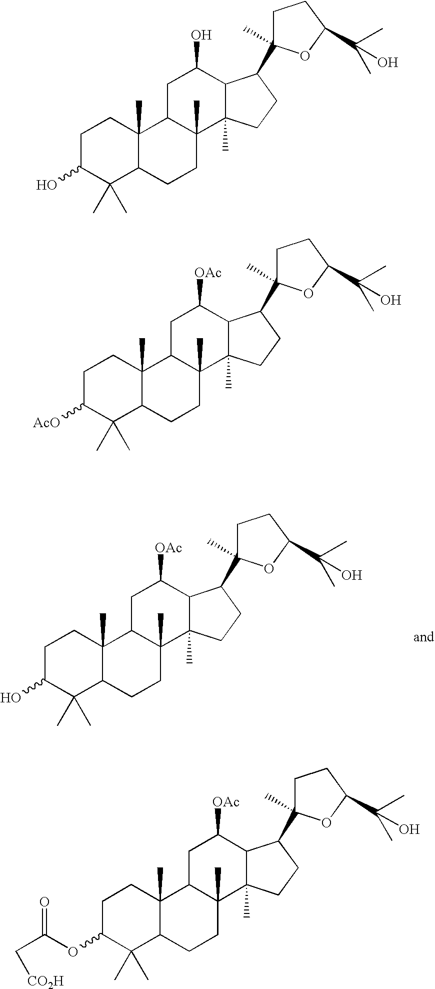
the method comprising the steps of:
(a) treating a compound having formula: 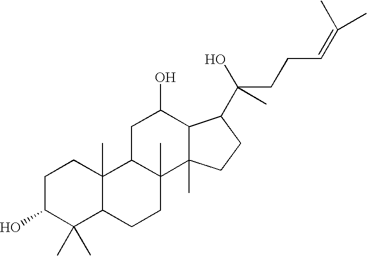
   with an oxidizing agent to form a compound having formula: 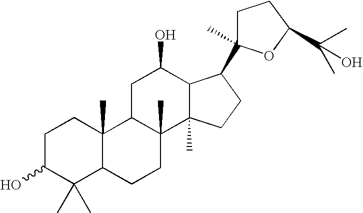
(b) treating the compound formed in step (a) with an acid anhydride or acid chloride to form a compound having formula: 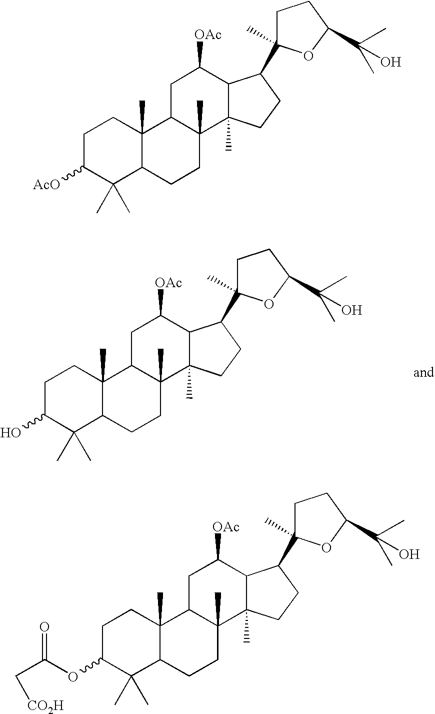
   In one embodiment, the starting compound is obtained from a plant, such as, for example, birch. In another embodiment, the oxidizing agent is MCPBA. In a further embodiment, the acid anhydride is (RCO)₂O.

Additionally, the present invention provides a method for treating or preventing a pathological condition in a subject, comprising administering a compound having the general formula: 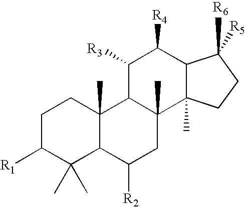
where R₁ is selected from the group consisting of α-OH, β-OH, α-O—X, β-O—X, α-R₇COO—, β-R₇COO—, α-R₇PO₃—, β-R₇PO₃—, α-NR₈R₉, β-NR₈R₉, ═O(oxo), ═NOH, ═NC(O)NHNH₂ and CH₂—X; where X is an alkyl or a carbohydrate containing one or more sugars or acylated derivatives thereof; R₇ is H, OH, an amino group, an alkenyl, aryl, or alkyl; R₈ is H, alkyl, aryl, acyl, or SO₂NHR₁₀; R₉ is H, alkyl, aryl, acyl, or SO₂NHR₁₀; R₁₀ is NH2, OH, alkyl, aryl, or cycloalkyl; ₂ is selected from the group consisting of H, OH, OAc and O—X, where X is a carbohydrate containing one or more sugars or acylated derivatives thereof, Ac═CH₃CO or acyl; 3 is selected from the group consisting of H, OH, O—X and OAc, where X is an alkyl or a carbohydrate containing one or more sugars or acylated derivatives thereof; R₄ is selected from the group consisting of H, OH, O—X and OAc, where X is an alkyl or a carbohydrate containing one or more sugars or acylated derivatives thereof; R5 is H or OH; and R6 is alkenyl, aryl, or alkyl. In one embodiment, the pathological condition is neurodegeneration, preferably, Alzheimer's disease and Aβ42-related disorder.

The present invention further provides a method for inhibiting β-amyloid production in subject, including inhibiting β-amyloid production in an in vitro context, comprising administering a compound having the general formula: 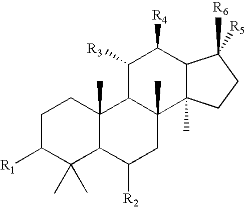
where R₁ is selected from the group consisting of α-OH, β-OH, α-O—X, β-O—X, α-R₇COO—, β-R₇COO—, α-R₇PO₃—, β-R₇PO₃—, α-NR₈R₉, β-NR₈R₉, ═O(oxo), ═NOH, ═NC(O)NHNH₂ and CH₂—X; where X is an alkyl or a carbohydrate containing one or more sugars or acylated derivatives thereof; R₇ is H, OH, an amino group, an alkenyl, aryl, or alkyl; R₈ is H, alkyl, aryl, acyl, or SO₂NHR₁₀; R₉ is H, alkyl, aryl, acyl, or SO₂NHR₁₀; R₁₀ is NH2, OH, alkyl, aryl, or cycloalkyl; 2 is selected from the group consisting of H, OH, OAc and O—X, where X is a carbohydrate containing one or more sugars or acylated derivatives thereof, Ac═CH₃CO or acyl; R₃ is selected from the group consisting of H, OH, O—X and OAc, where X is an alkyl or a carbohydrate containing one or more sugars or acylated derivatives thereof; R₄ is selected from the group consisting of H, OH, O—X and OAc, where X is an alkyl or a carbohydrate containing one or more sugars or acylated derivatives thereof; R5 is H or OH; and R₆ is alkenyl, aryl, or alkyl.

Additional aspects of the present invention will be apparent in view of the description that follows.

### DESCRIPTION OF THE FIGURES

FIG. 1 shows that compounds D5, D6, D10, D11, D12, D15 exhibited Aβ-lowering activities. In contrast, some of the compounds selectively potentiated Aβ42 production (e.g., D1, D2, D3, D7 and D9). (D1=20(S), 24(R)-epoxydammarane-3β, 25-diol; D2=Dammar-24-ene-3β,20(S)-diol; D3=reduction mixture of dammar resin by NaBH₄; D5=Dammar resin mixture; D6=dipterocarpol; D7=t-butylhydroperoxide-oxidized products of D3. D8=3-acetyl Dammar-24-ene-3β,20(S)-diol; D9=3-acetyl 20(S),24(R)-epoxydammarane-3β,25-diol; D10=20(S),24(R)-epoxydammarane-3 cc, 25-diol; D11=Dammar-24-ene-3α,20(S)-diol; D12=tetraacetyl 20-hydroxydammar-24-ene-3-yl-glucopyranoside; D13=20-hydroxydammar-24-ene-3-yl-glucopyranoside; D15=20(S), 24(R)-epoxydammarane-3-oxo, 25-ol.

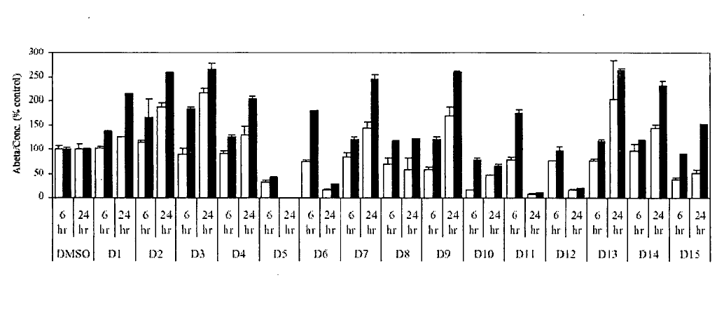

### DETAILED DESCRIPTION OF THE INVENTION

As used herein and in the appended claims, the singular forms “a,” “an” and “the” include plural references unless the content clearly dictates otherwise. Thus, for example, reference to “an agent” includes a plurality of such agents and reference to “the compound” is a reference to one or more compounds and equivalents thereof known to those skilled in the art, and so forth. All publications, patent applications, patents and other references mentioned herein are incorporated by reference in their entirety.

In accordance with the present invention, compounds and methods for treating Alzheimer's disease, neurodegeneration and for modulating the production of amyloid-beta protein (Aβ) are provided.

In one aspect, the present invention provides a compound having the general formula: 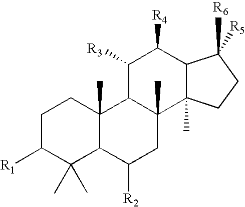
where R₁ is selected from the group consisting of α-OH, β-OH, α-O—X, β-O—X, α-R₇COO—, β-R₇COO—, α-R₇PO₃—, β-R₇PO₃—, α-NR₈R₉, β-NR₈R₉, ═O(oxo), ═NOH, ═NC(O)NHNH₂ and CH₂—X; where X is an alkyl or a carbohydrate containing one or more sugars or acylated derivatives thereof; R₇ is H, OH, an amino group, an alkenyl, aryl, or alkyl; R₈ is H, alkyl, aryl, acyl, or SO₂NHR₁₀; R₉ is H, alkyl, aryl, acyl, or SO₂NHR₁₀; R₁₀ is NH2, OH, alkyl, aryl, or cycloalkyl; R₂ is selected from the group consisting of H, OH, OAc and O—X, where X is a carbohydrate containing one or more sugars or acylated derivatives thereof, Ac═CH₃CO or acyl; R₃ is selected from the group consisting of H, OH, O—X and OAc, where X is an alkyl or a carbohydrate containing one or more sugars or acylated derivatives thereof; R₄ is selected from the group consisting of H, OH, O—X and OAc, where X is an alkyl or a carbohydrate containing one or more sugars or acylated derivatives thereof; R5 is H or OH; and R6 is alkenyl, aryl, or alkyl. In one embodiment of the present invention, R₇ further contains oxygen, nitrogen or phosphorus. In another embodiment of the invention R₆ further contains a functional group selected from the group consisting of hydroxyl, ether, ketone, oxime, hydrazone, imine and Schiff base. In still another embodiment of the present invention, the sugar is selected from the group consisting of Glc, Ara(pyr), Ara(fur), Rha and Xyl and acylated derivatives thereof. In a further embodiment, R₄ is selected from the group consisting of: 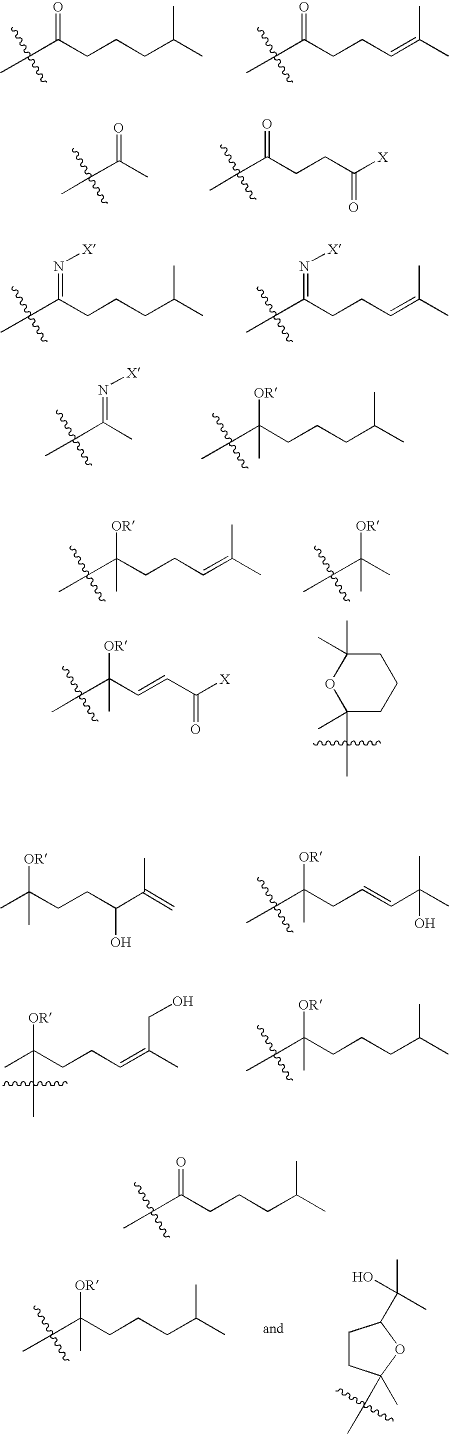
where the configuration of any stereo center is R or S; X is OR or NR, X′ is alkyl, OR or NR; and where R is alkyl or aryl; and R′ is H, alkyl or acyl.

In a preferred embodiment, the compounds of the invention include but are not limited to:

Dammar-24-ene-3α,20(S)-diol; Dammar-24-ene-3β,20(S)-diol; 3-[3-(3,4-dihydroxyphenyl)-2-propenoate]-(3α,20S)-Dammar-24-ene-3,20-diol; 3-[3-(3,4-dihydroxyphenyl)-2-propenoate]-(3β,20S)-Dammar-24-ene-3,20-diol; 3-[3-(4-hydroxyphenyl)-2-propenoate]-(3β,20S)-Dammar-24-ene-3,20-diol; 3-[3-(4-hydroxyphenyl)-2-propenoate]-(3α,20S)-Dammar-24-ene-3,20,26-triol; 3-[3-(3,4-dihydroxyphenyl)-2-propenoate]-(3β,20S)-Dammar-24-ene-3,20,26-triol; Dammare-23-ene-3α,20(S), 25-triol; Dammare-25-ene-3α,20(S), 24-triol; (3α)-20-hydroxydammar-24-ene-3-yl-D-Glc (1-2)-glucopyranoside; (3β)-20-hydroxydammar-24-ene-3-yl-D-Glc (1-2)-glucopyranoside; 20,24-epoxy-(3α,24R)-Dammarane-3,25-diol; 20,24-epoxy-(3β,24R)-Dammarane-3,25-diol; 20,24-epoxy-3-acetyl-(3α,24R)-Dammarane-3,25-diol; 20,24-epoxy-3-acetyl-(3β,24R)-Dammarane-3,25-diol; 3α-malonyl-20(S), 24(R)-epoxydammarane-25-ol; 3-[3-(3,4-dihydroxyphenyl)-2-propenoate]-20,24-epoxy-(3β,24R)-Dammarane-3,25-diol; 20(S), 24(R)-epoxydammarane-25-ol-3-one; 3-phosphoryl-(3β,20s)-Dammar-24-ene-3,20-diol; 3-amino-Dammar-24-ene-20(S)-ol; {[(20-hydroxydammar-24-ene-3-yl-) carbamoyl]-methoxy}-acetic acid; 3-(NH₂SO₂NH)-Dammar-24-ene-20-ol; 3(C₅H₁₀NHSO₂NH)-Dammar-24-ene-20-ol; (3α,12b,20S)-Dammar-24-ene-3,12,20-triol; (3α,12b,20S)-Dammar-25-ene-3,12,20,24-tetraol; 12,20-dihydroxy-(12β,20S)-Dammar-24-en-3-one; 12β-acetyloxy-20(S)-hydroxy-Dammar-24-en-3-one; (3α,12β,20S)-Dammar-25-ene-3,12,20,24-tetraol; (3α,12β,20S)-Dammar-23-ene-3,12,20,25-tetraol; (3α,12β)-20-hydroxydammar-24-ene-3,12-diyl-bis-β-D-Glucopyranoside; (3β,12β)-20-hydroxydammar-24-ene-3,12-diyl-bis-β-D-Glucopyranoside; (3β,12β,20S)-12-acetyl-Dammar-24-ene-3,12,20-triol; (3α,12β,20S)-12-acetyl-Dammar-24-ene-3,12,20-triol; (3α,12β,20S)-3,12-diacetate-Dammar-24-ene-3,12,20-triol; 12-acetyl 3-(hydrogen propanedioate)-(3α,12β)-Dammar-24-ene-3,12,20-triol; 12-acetyl-3-(methyl propanedioate)-(3α,12β)-Dammar-24-ene-3,12,20-triol; (3α,12β)-20-hydroxydammar-24-ene-3-yl-D-Glc (1-2) glucopyranoside; 12,20,25-trihydroxy-Dammar-23-en-3-one; (3α, 12β)-Dammar-23-ene-3,12,20,25-tetrol; 12-acetyl 3-(hydrogen propanedioate)-(3α,12β,23E)-Dammar-23-ene-3,12,20,25-tetrol; 12-acetyl 3-(methyl propanedioate)-(3α,12β, 23E)-Dammar-23-ene-3,12,20,25-tetrol; 20(S), 24(R)-epoxy-dammarane-3α,12β,25-triol; 20(S), 24(R)-epoxy-dammarane-3β,12β,25-triol; 3α,12β-diacetoxy-20(S), 24(R)-epoxy-dammarane-25-ol; 12β-acetoxy-20(S), 24(R)-epoxydammarane-3α,25-diol; 20,24-epoxy-12-acetyl-3-(hydrogen propanedioate)-(3α,12β,24R)-Dammarane-3,12,25-triol; 20,24-epoxy-12-acetyl 3-(methyl propanedioate)-(3α,12β,24R)-Dammarane-3,12,25-triol; 3-NH₂SO₂NH-Dammar-24-ene-12,20S-diol; 3-amino-Dammar-24-ene-12,20S-diol; (3α,17α,20S)-Dammar-24-ene-3,17,20-triol; 12-acetyl-(3α,12β,20S)-Dammar-24-ene-3,12,17,20-tetrol; (3α,12β,17R,20S)-Dammar-25-ene-3,12,17,20,24-pentol; 12-acetyl-3-(hydrogen propanedioate)-(3α,12β)-Dammar-24-ene-3,12,17,20-tetrol; 12-acetyl-3-(methyl propanedioate)-(3α,12β)-Dammar-24-ene-3,12,17,20-tetrol; 20,24-epoxy-(3α, 24R)-Dammarane-3,17,25-triol; 20(S), 24(R)-epoxy-dammarane-17α,25-diol-3-one; 20,24-epoxy-(3α,12β,20S,24R)-Dammarane-3,12,17,25-tetrol; 20,24-epoxy-(3β,12β,20S,24S)-Dammarane-3,12,17,25-tetrol; 20,24-epoxy-12-acetyl-(3a,12b,24R-Dammarane-3,12,17,25-tetrol; (3α,12β,24R)-12-(acetyloxy)-20,24-epoxy-17,25-dihydroxydammaran-3-yl-β-D-Glucopyranoside, 6-acetate; 3α,11α-diacetoxy-20(S), 24(R)-epoxydammarane-17α,25-diol; (3β,11α,20S)-Dammar-24-ene-3,11,20-triol; (3β,11α)-11,20-dihydroxydammar-24-en-3-yl-β-D-Glucopyranoside; 3,11-diacetate, (3β,11α)-Dammar-24-ene-3,11,20-triol; (3β,11α)-11,20-dihydroxydammar-24-en-3-yl-β-D-Glucopyranoside, 2-acetate; 20,24-epoxy-(3α,11α,24R)-Dammarane-3,11,25-triol; 20(S), 24(R)-epoxy-dammmaraen-3β,11α-25-triol; 11α-acetoxy-20(S), 24(R)-epoxydammarane-3α,25-diol; 11α-acetoxy-20(S), 24(R)-epoxydammarane-3α,25-diol; 20,24-epoxy-11,25-dihydroxy-(11α,24R)-Dammaran-3-one; 3-acetyl-20,24-epoxy-(3α,11α,24R)-Dammarane-3,11,25-triol; 3α,11α-diacetoxy-20(S), 24(R)-epoxydammarane-25-ol; 3α,11α-diacetoxy-20(S), 24(R)-epoxydammarane-25-ol; (3β,11α,24R)-20,24-epoxy-11,25-dihydroxydammaran-3-yl-β-D-Glucopyranoside; (3β,11β,24R)-20,24-epoxy-11-acetyloxy-25-hydroxydammaran-3-yl-β-D-Glucopyranoside, 2-acetate; (3β,11α,24R)-20,24-epoxy-11,25-dihydroxydammaran-3-yl-β-D-Glucopyranoside, 2-acetate; 3-O-acetyl-20(S)-dammar-24-ene-3β,6α,20,26-tetraol 26-O-β-D-glucopyranoside; 3-oxo-20(S)-dammar-24-ene-6α,20,26-triol 26-O-β-D-glucopyranoside; 20(S)-dammar-24-ene-3β,6α,20,26-tetraol 26-O-β-D-glucopyranoside; 20(S)-dammar-24-ene-3β,20,26-triol 3,26-di-O-β-D-glucopyranoside; 3-oxo-20(S)-dammar-24-ene-6α,20,21,26-tetraol 26-O-β-D-glucopyranoside; 20(S)-dammar-24-ene-3β,6α,20,21,26-pentaol 26-O-β-D-glucopyranoside; 20(S)-dammar-24-ene-3β,6α,20,26-tetraol 3,26-di-O-β-D-glucopyranoside; and 20(S)-dammar-24-ene-3β,6α,20,26-tetraol-3-O-β-sophoroside-26-O-β-D-glucopyrano side.

As disclosed herein, the compounds are dammaranes and their analogues. The dammaranes of the present invention may be chemically associated with carbohydrates including, but not limited to, glucopyranosyl, arabinopyranosyl, arabinofuranosyl and rhamnopyranosyl. The dammaranes of the present invention may be isolated dammarane compounds or isolated and further synthesized dammaranes. The isolated dammaranes of the present invention can be further synthesized using processes including, but not necessarily limited to, heat, light, chemical, enzymatic or other synthesis processes generally known to the skilled artisan.

The present invention also provides a method for the synthesis of a compound having formula: 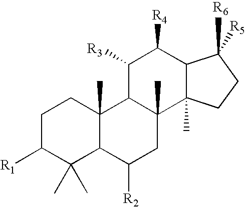
which comprises the steps of:
(a) treating a compound having formula: 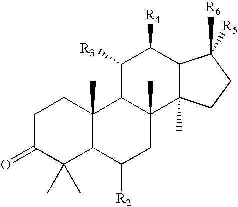
   with a reducing agent, followed by an appropriate oxidizing agent to form a compound having formula: 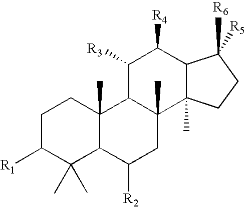
   where R₁ is selected from the group consisting of α-OH, β-OH, α-O—X, β-O—X, α-R₇COO—, β-R₇COO—, α-R₇PO₃—, β-R₇PO₃—, α-NR₈R₉, β-NR₈R₉, ═O(oxo), ═NOH, ═NC(O)NHNH₂, and CH₂—X; where X is an alkyl or a carbohydrate containing one or more sugars or acylated derivatives thereof; R₇ is H, OH, an amino group, an alkenyl, aryl, or alkyl; R₈ is H, alkyl, aryl, acyl, or SO₂NHR₁₀; R₉ is H, alkyl, aryl, acyl, or SO₂NHR₁₀; R₁₀ is NH₂, OH, alkyl, aryl, or cycloalkyl; R₂ is selected from the group consisting of H, OH, OAc, and O—X, where X is a carbohydrate containing one or more sugars or acylated derivatives thereof, Ac═CH₃CO or acyl; R₃ is selected from the group consisting of H, OH, O—X and OAc, where X is an alkyl or a carbohydrate containing one or more sugars or acylated derivatives thereof; R₄ is selected from the group consisting of H, OH, O—X and OAc, where X is an alkyl or a carbohydrate containing one or more sugars or acylated derivatives thereof; R5 is H or OH; and R6 is alkenyl, aryl, or alkyl.

The present invention further provides a method for the synthesis of a compound having formula: 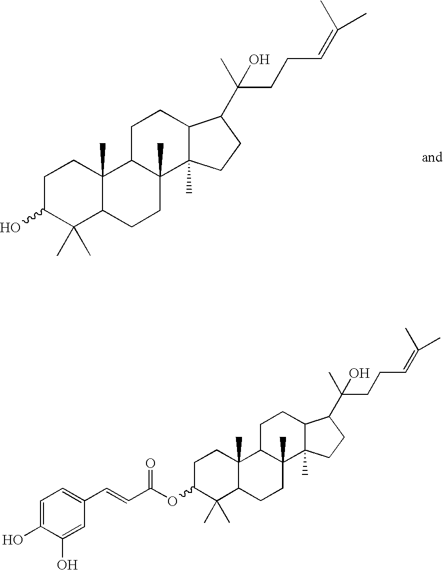
said method comprising the steps of:
(a) treating a compound having formula: 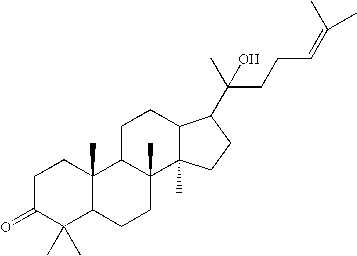
   with a reducing agent, to form a compound having formula: 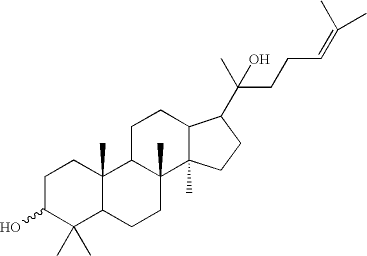
(b) treating the compound formed in step (a) with an esterificating agent followed by a hydrolyzing agent, to form a compound having formula: 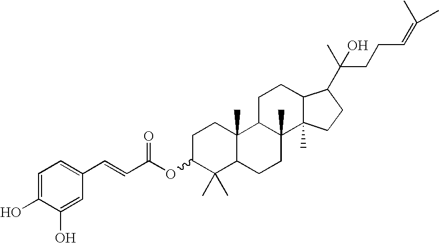
   In one embodiment, the reducing agent is NaBH₄. In another embodiment, the esterificating agent is di-O-acetylcaffeoyl chloride and the hydrolyzing agent is NaHCO₃.

Additionally, the invention provides a method for the synthesis of a compound having formula: 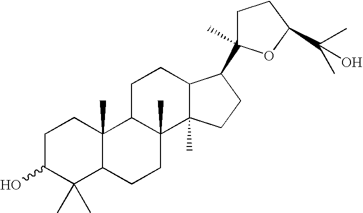
the method comprising the steps of:
(a) treating a compound having formula: 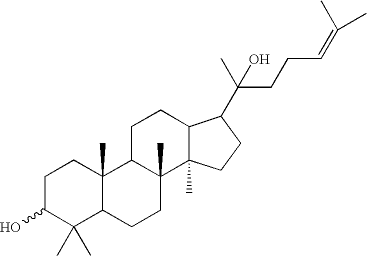
   with an oxidizing agent, to form a compound having formula: 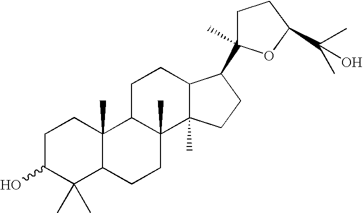
   (b) treating the compound formed in step (a) with an esterificating agent to form a compound having formula: 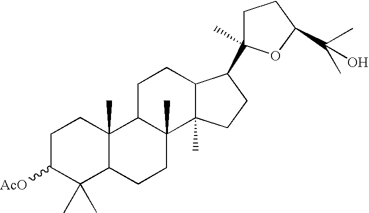
   In one embodiment, the oxidizing agent is MCPBA. In another embodiment, the esterificating agent is Ac₂O.

In still another aspect, the present invention provides a method for the synthesis of a compound having formula: 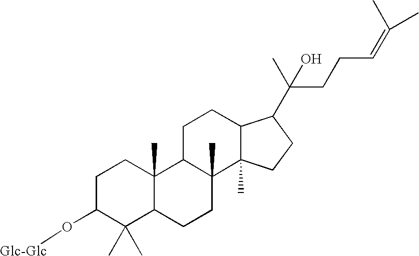
the method comprising the steps of:
(a) treating a compound having formula: 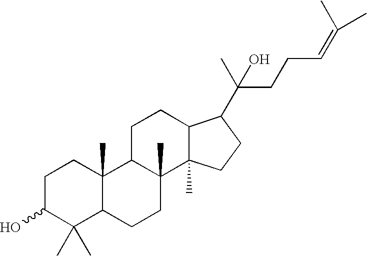
   with an appropriate bromonated sugar and an oxidizing agent, followed by deprotection of the acetyl groups to form a compound having formula: 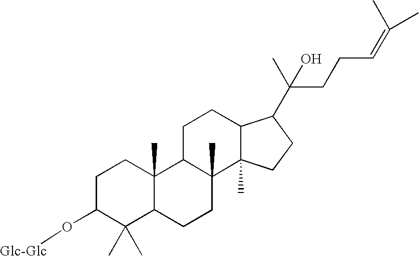
   In one embodiment, the oxidizing agent is Ag2O. In yet another embodiment, the compound is deprotected using NaOMe.

The present invention further provides a method for the synthesis of a compound having formula: 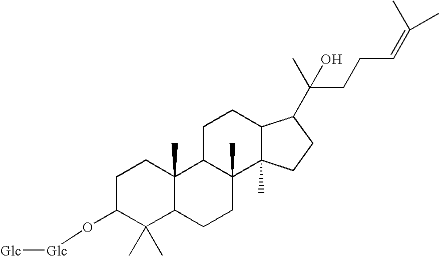
the method comprising the steps of:
(a) treating a compound having formula: 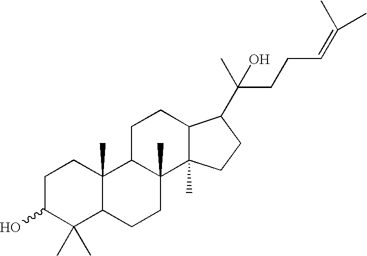
   with an appropriate bromonated sugar and an oxidizing agent, followed by deprotection of the acetyl groups to form a compound having formula: 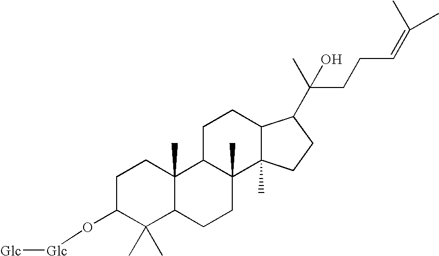
   In one embodiment, the oxidizing agent is Ag2O. In another embodiment, the compound is deprotected using NaOMe.

The present invention also provides a method for the synthesis of a compound having the formula: 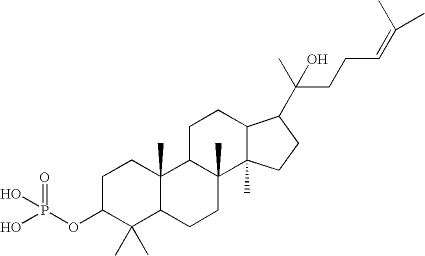
The method comprising the steps of:
(a) treating a compound having formula: 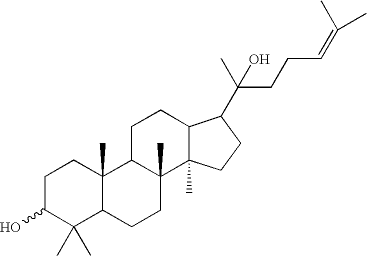
   with a dibenzylphosphoric agent and a base to form a compound having formula: 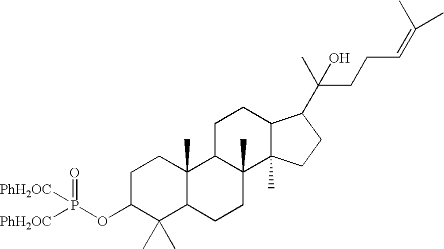
(b) treating the compound formed in step (a) with a hydrogenating agent to form a compound having formula: 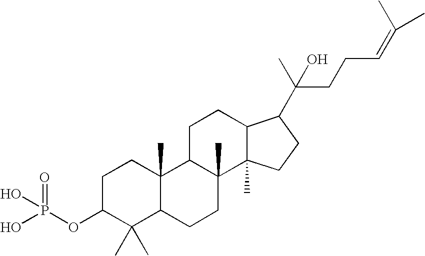
   In one embodiment, the dibenzylphosphoric agent is (PhCH₂O)₂POCl. In another embodiment, the hydrogenating agent is H₂/Pd—C.

The present invention further provides a method for the synthesis of a compound having the formula: 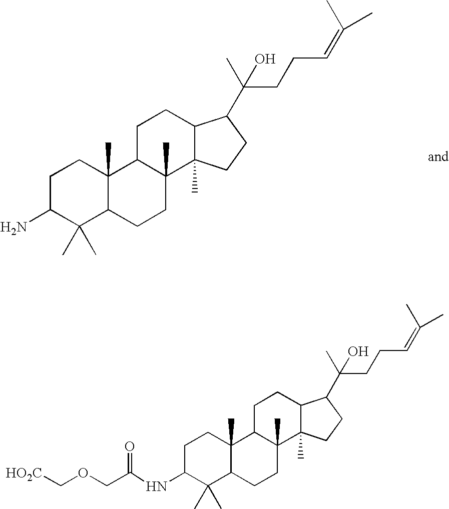
the method comprising the steps of:
(a) treating a compound having formula: 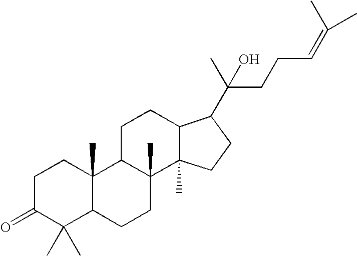
   with a reducing agent to form a compound having formula: 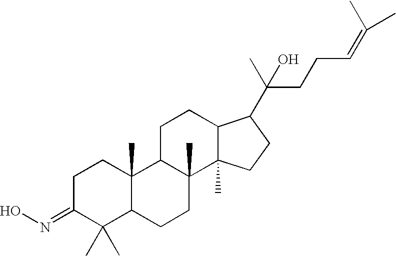
(b) treating the compound formed in step (a) with an additional reducing agent to form a compound having formula: 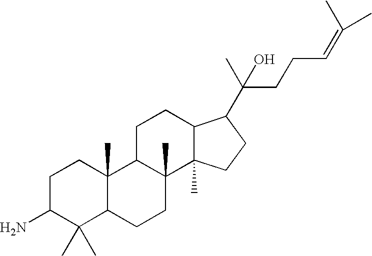
(c) treating the compound formed in step (b) with diglylcolic anhydride in the presence of a base to form a compound having formula: 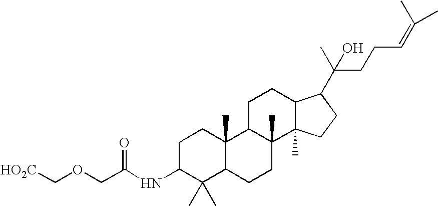
   In one embodiment, the reducing agent of step (a) is NH2OH. In another embodiment, the reducing agent of step (b) is Na.

The present invention also provides a method for the synthesis of a compound having the formula: 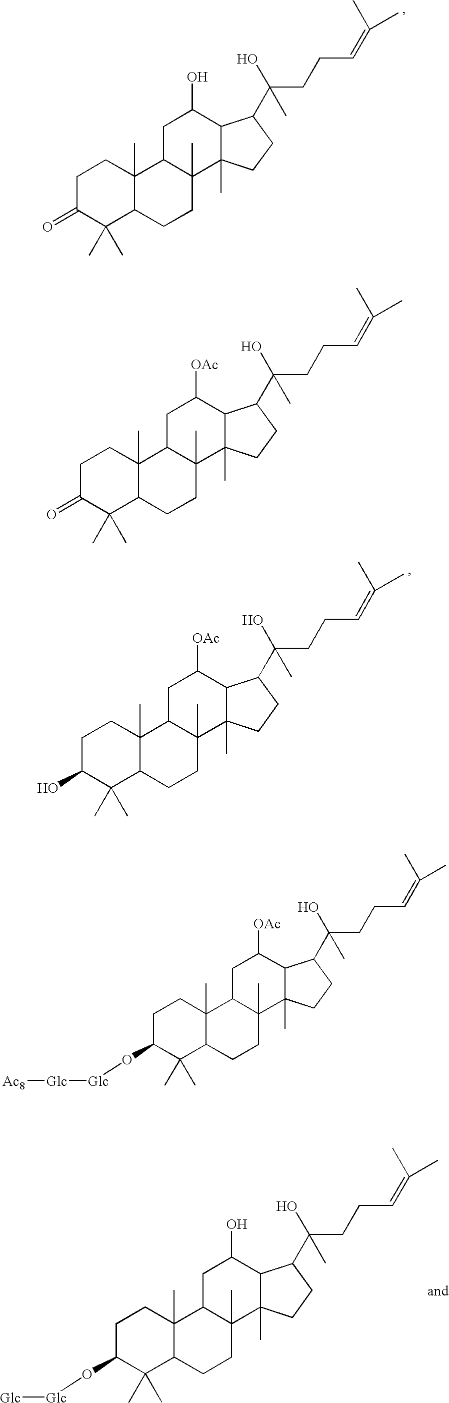 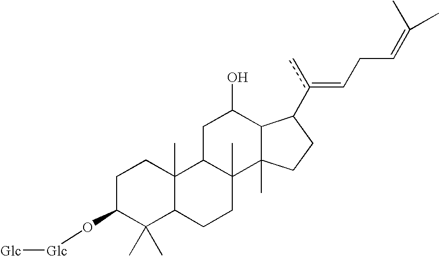
the method comprising the steps of:
(a) treating a compound having formula: 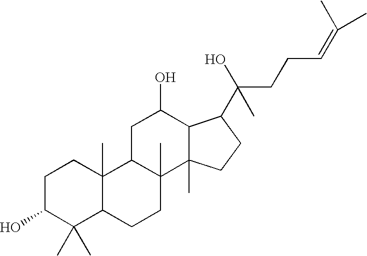
   with an oxidizing agent to form a compound having formula: 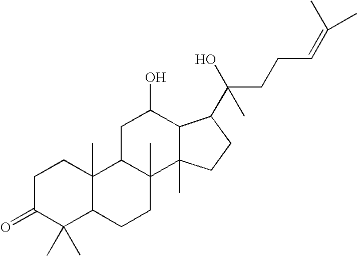
(b) treating the compound formed in step (a) with an acetylating agent to form a compound having formula: 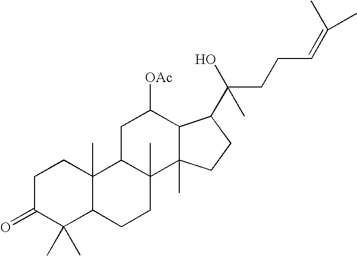
(c) treating the compound formed in step (b) with a reducing agent to form a compound having formula: 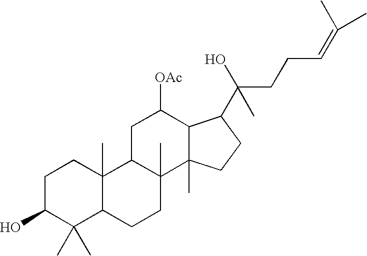
(d) treating the compound formed in step (c) with Ac₈-Glc-Glc-Br in the presence of Ag₂O to form a compound having formula: 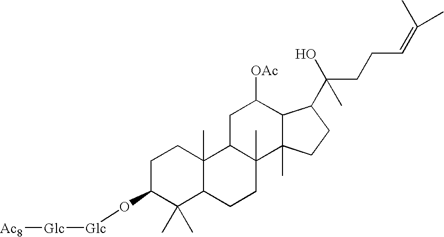
(e) treating the compound formed in step (d) with a deacetylating agent to form a compound having formula: 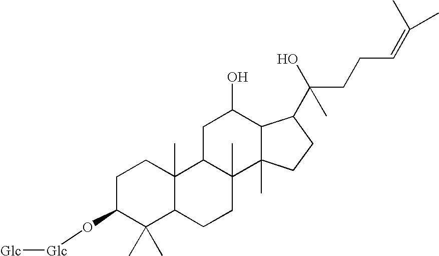
(f) treating the compound formed in step (e) with a dehydrating agent to form a compound having formula: 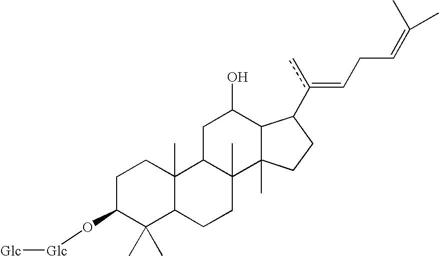
   In one embodiment, the starting compound is obtained from a plant, such as, for example, birch. In another embodiment, the oxidizing agent is CrO₃ in pyridine. In a further embodiment, the acetylating agent is Ac₂O in pyridine. In another embodiment, the reducing agent is NaBH₄. In another embodiment, the dehydrating agent is mesyl chloride and triethylamine. In yet another embodiment, the deacetylating agent is NaOMe.

The present invention also provides a method for the synthesis of a compound having the formula: 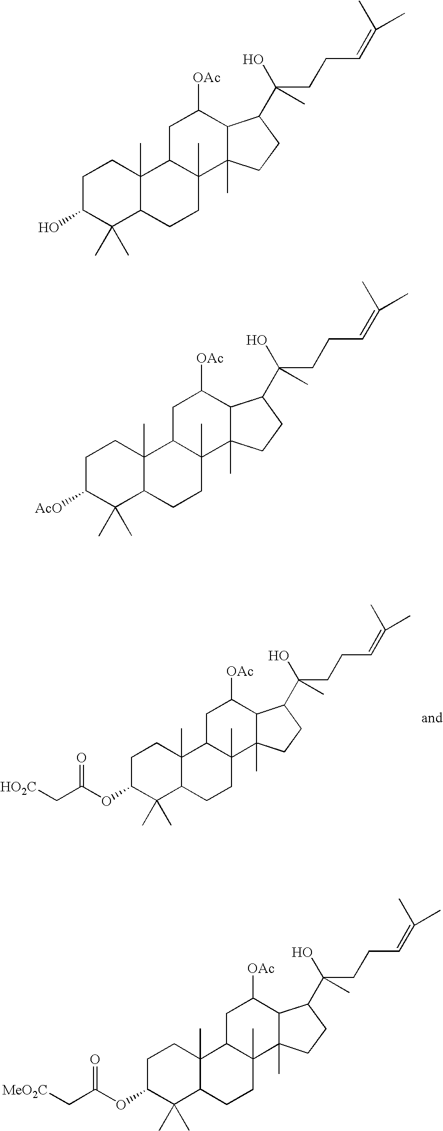
the method comprising the steps of:
(a) treating a compound having formula: 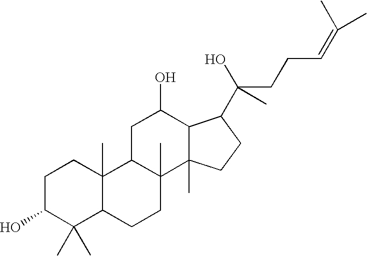
   with AC₂O to form a compound having formula: 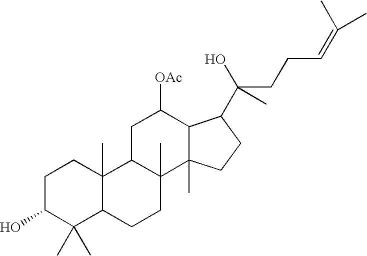
(b) treating the compound formed in step (a) with Ac₂O to form a compound having formula: 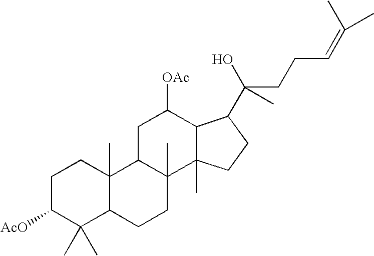
(c) treating the compound formed in step (a) with CH₂(COCl)₂ to form a compound having formula: 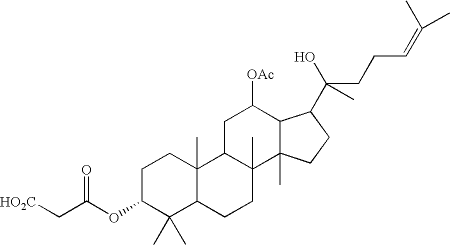
(d) treating the compound formed in step (c) with CH₂N₂ to form a compound having formula: 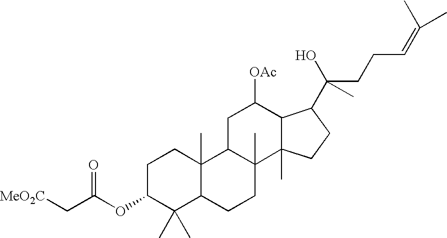
   In one embodiment, the starting compound is obtained from a plant, such as, for example, birch.

The present invention additionally provides a method fro the synthesis of a compound having the formula: 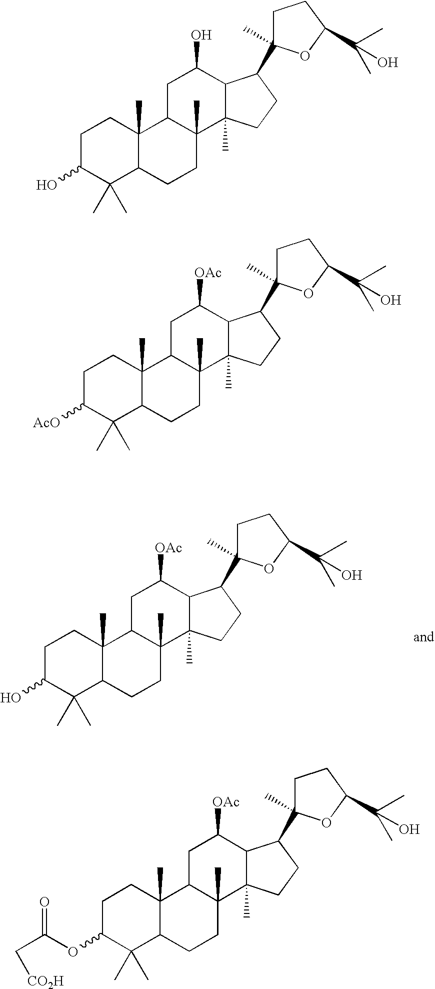
the method comprising the steps of:
(a) treating a compound having formula: 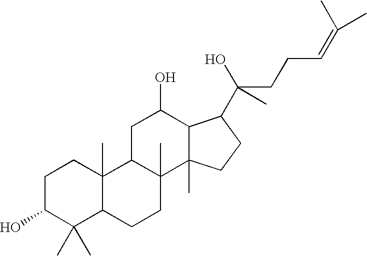
   with an oxidizing agent to form a compound having formula: 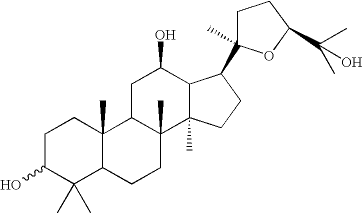
(b) treating the compound formed in step (a) with an acid anhydride or acid chloride to form a compound having formula: 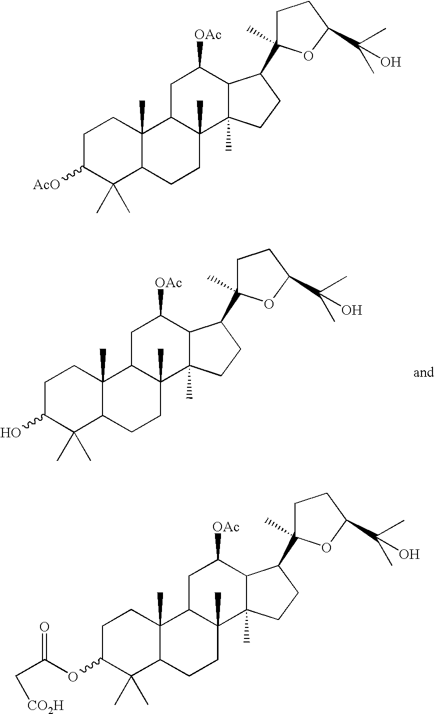
   In one embodiment, the starting compound is obtained from a plant, such as, for example, birch. In another embodiment, the oxidizing agent is MCPBA. In a further embodiment, the acid anhydride is (RCO)₂O.

Additionally, the present invention provides a method for treating or preventing a pathological condition in a subject, comprising administering a compound having the general formula: 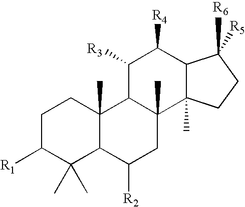
where R₁ is selected from the group consisting of α-OH, β-OH, α-O—X, β-O—X, α-R₇COO—, β-R₇COO—, α-R₇PO₃—, β-R₇PO₃—, α-NR₈R₉, β-NR₈R₉, ═O(oxo), ═NOH, ═NC(O)N NH₂ and CH₂—X; where X is an alkyl or a carbohydrate containing one or more sugars or acylated derivatives thereof; R₇ is H, OH, an amino group, an alkenyl, aryl, or alkyl; R₈ is H, alkyl, aryl, acyl, or SO₂NHR₁₀; R₉ is H, alkyl, aryl, acyl, or SO₂NHR₁₀; R₁₀ is NH2, OH, alkyl, aryl, or cycloalkyl; R₂ is selected from the group consisting of H, OH, OAc and O—X, where X is a carbohydrate containing one or more sugars or acylated derivatives thereof, Ac═CH₃CO or acyl; R₃ is selected from the group consisting of H, OH, O—X and OAc, where X is an alkyl or a carbohydrate containing one or more sugars or acylated derivatives thereof; R₄ is selected from the group consisting of H, OH, O—X and OAc, where X is an alkyl or a carbohydrate containing one or more sugars or acylated derivatives thereof; R5 is H or OH; and R6 is alkenyl, aryl, or alkyl. In one embodiment, the pathological condition is neurodegeneration, preferably, Alzheimer's disease and Aβ42-related disorder.

The present invention further provides a method for inhibiting β-amyloid production in subject, including inhibiting β-amyloid production in an in vitro context, comprising administering a compound having the general formula: 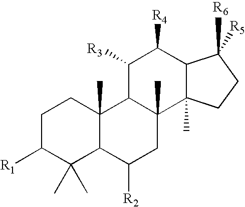
where R₁ is selected from the group consisting of α-OH, β-OH, α-O—X, β-O—X, α-R₇COO—, β-R₇COO—, α-R₇PO₃—, β-R₇PO₃—, α-NR₈R₉, β-NR₈R₉, ═O(oxo), ═NOH, ═NC(O)NHNH₂ and CH₂—X; where X is an alkyl or a carbohydrate containing one or more sugars or acylated derivatives thereof; R₇ is H, OH, an amino group, an alkenyl, aryl, or alkyl; R₈ is H, alkyl, aryl, acyl, or SO₂NHR₁₀; R₉ is H, alkyl, aryl, acyl, or SO₂NHR₁₀; R₁₀ is NH2, OH, alkyl, aryl, or cycloalkyl; R₂ is selected from the group consisting of H, OH, OAc and O—X, where X is a carbohydrate containing one or more sugars or acylated derivatives thereof, Ac═CH₃CO or acyl; R₃ is selected from the group consisting of H, OH, O—X and OAc, where X is an alkyl or a carbohydrate containing one or more sugars or acylated derivatives thereof; R₄ is selected from the group consisting of H, OH, O—X and OAc, where X is an alkyl or a carbohydrate containing one or more sugars or acylated derivatives thereof; R5 is H or OH; and R6 is alkenyl, aryl, or alkyl.

The present invention further provides a method for the synthesis of a compound having formula: 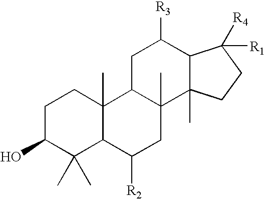
wherein the method comprises the steps of:
(a) treating a compound having formula: 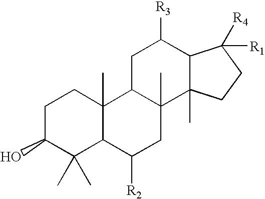
   with an oxidizing agent, to form a compound having formula: 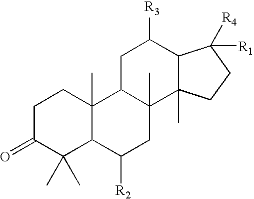
(b) treating the compound formed in step (a) with a reducing agent, to form a compound having formula: 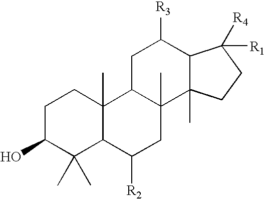
   wherein R₁ is H or OH; R₂ is selected from the group consisting of H, OH, OAc, and O—X, wherein X is a carbohydrate containing one or more sugars or acylated derivatives thereof; R₃ is selected from the group consisting of H, OH, and OAc; and R₄ is alkenyl, aryl, or alkyl. In one embodiment, the oxidizing agent is chromic anhydride and the reducing agent is NaBH₄.

The starting material, i.e., the compound having formula: 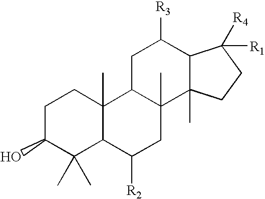
particularly, betulafolienetriol, may be obtained from plants including, without limitation, common birch. The extracts of these plants are rich sources of betulafolienetriol and are desired starting materials for making dammaranes because they cost significantly less than ginseng.

The present invention also provides a method for the synthesis of a compound having formula: 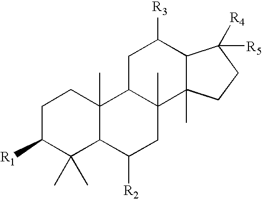
wherein the method comprises the steps of:
(a) treating a compound having formula: 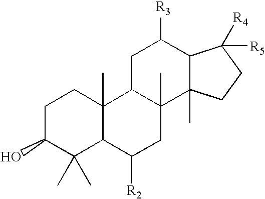
   with an oxidizing agent, to form a compound having formula: 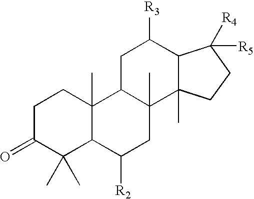
(b) treating the compound formed in step (a) with a reducing agent, to form a compound having formula: 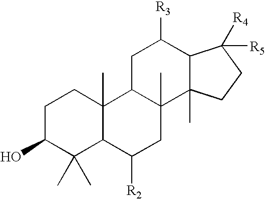
(c) optionally, treating the compound formed in step (b) with protected R₁ derivative, to form a compound having formula: 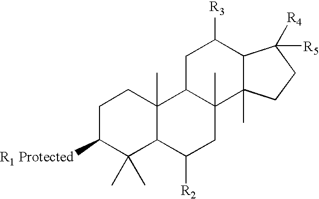
(d) treating the compound formed in step (c) with deprotection agent, to form a compound having formula: 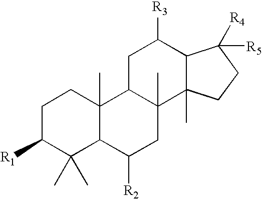
   wherein R1 is selected from the group consisting of α-OH, β-OH, α-O—X, β-O—X, α-R₆COO—, β-R₆COO—, α-R₆PO₃—, and β-R₆PO₃—, wherein X is a carbohydrate containing one or more sugars or acylated derivatives thereof, and R₆ is alkenyl, aryl, or alkyl I; R₂ is selected from the group consisting of H, OH, OAc, and O—X, wherein X is a carbohydrate containing one or more sugars or acylated derivatives thereof; R₃ is selected from the group consisting of H, OH, and OAc; R₄ is alkenyl, aryl, or alkyl II; and R₅ is H or OH. The alkyl I group may further contain oxygen, nitrogen, or phosphorus; and the alkyl II group may further contain a function group, such as hydroxyl, ether, ketone, oxime, hydrazone, imine, and Schiff base. In one embodiment, the oxidizing agent is chromic anhydride and the reducing agent is NaBH₄. In another embodiment, the protected R₁ derivative is a protected R₁ halogen derivative. For example, the protected R₁ derivative may be protected by an Ac₈-group. The protected R₁ group may be deprotected using agents such as NaOMe.

Additionally, the present invention provides dammarane compositions for use in modulating amyloid-beta production in a subject, treating or preventing Alzheimer's disease and treating or preventing neurodegeneration comprising a mixture of isolated or isolated and further synthesized dammaranes.

The present invention provides methods and pharmaceutical compositions for use in decreasing amyloid-beta production, comprising use of a pharmaceutically-acceptable carrier and a dammarane compound. Examples of acceptable pharmaceutical carriers, formulations of the pharmaceutical compositions, and methods of preparing the formulations are described herein. The pharmaceutical compositions may be useful for administering the dammarane compounds of the present invention to a subject to treat a variety of disorders, including neurodegeneration and/or its associated symptomology, as disclosed herein. The dammarane compound is provided in an amount that is effective to treat the disorder (e.g., neurodegeneration) in a subject to whom the pharmaceutical composition is administered. The skilled artisan, as described above, may readily determine this amount. In one embodiment, the present invention provides a method for inhibiting β-amyloid production in a subject, comprising administering a compound having the general formula: 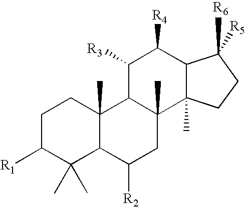
where R₁ is selected from the group consisting of α-OH, β-OH, α-O—X, β-O—X, α-R₇COO—, β-R₇COO—, α-R₇PO₃—, β-R₇PO₃—, α-NR₈R₉, β-NR₈R₉, ═O(oxo), ═NOH, ═NC(O)NHNH₂ and CH₂—X; where X is an alkyl or a carbohydrate containing one or more sugars or acylated derivatives thereof; R₇ is H, OH, an amino group, an alkenyl, aryl, or alkyl; R₈ is H, alkyl, aryl, acyl, or SO₂NHR₁₀; R₉ is H, alkyl, aryl, acyl, or SO₂NHR₁₀; R₁₀ is NH2, OH, alkyl, aryl, or cycloalkyl; R₂ is selected from the group consisting of H, OH, OAc and O—X, where X is a carbohydrate containing one or more sugars or acylated derivatives thereof, Ac═CH₃CO or acyl; R₃ is selected from the group consisting of H, OH, O—X and OAc, where X is an alkyl or a carbohydrate containing one or more sugars or acylated derivatives thereof; R₄ is selected from the group consisting of H, OH, O—X and OAc, where X is an alkyl or a carbohydrate containing one or more sugars or acylated derivatives thereof; R5 is H or OH; and R6 is alkenyl, aryl, or alkyl. As used herein, the term “subject” includes, for example, an animal, e.g., human, rat, mouse, rabbit, dog, sheep and cow, as well as an in vitro system, e.g., a cultured cell, tissue and organ.

The present invention also provides a method for treating neurodegeneration in a subject in need of treatment, by contacting cells (preferably, cells of the CNS) in the subject with an amount of a dammarane compound or composition effective to decrease amyloid-beta production in the cells, thereby treating the neurodegeneration. Examples of neurodegeneration which may be treated by the method of the present invention include, without limitation, Alzheimer's disease, amyotrophic lateral sclerosis (Lou Gehrig's disease), Binswanger's disease, corticobasal degeneration (CBD), dementia lacking distinctive histopathology (DLDH), frontotemporal dementia (FTD), Huntington's chorea, multiple sclerosis, myasthenia gravis, Parkinson's disease, Pick's disease, and progressive supranuclear palsy (PSP). In a preferred embodiment of the present invention, the neurodegeneration is Alzheimer's disease (AD) or sporadic Alzheimer's disease (SAD). In a further embodiment of the present invention, the Alzheimer's disease is early-onset familial Alzheimer's disease (FAD). The skilled artisan can readily determine when clinical symptoms of neurodegeneration have been ameliorated or minimized.

The present invention also provides a method for treating or preventing a pathological condition, such as neurodegeneration and Aβ42-related disorder, in a subject in need of treatment, comprising administering to the subject one or more dammarane compounds in an amount effective to treat the neurodegeneration. The Aβ42-related disorder may be any disorder caused by Aβ42 or has a symptom of aberrant Aβ42 accumulation. As used herein, the phrase “effective to treat the neurodegeneration” means effective to ameliorate or minimize the clinical impairment or symptoms of the neurodegeneration. For example, where the neurodegeneration is Alzheimer's disease, the clinical impairment or symptoms of the neurodegeneration may be ameliorated or minimized by reducing the production of amyloid-beta and the development of senile plaques and neurofibrillary tangles, thereby minimizing or attenuating the progressive loss of cognitive function. The amount of inhibitor effective to treat neurodegeneration in a subject in need of treatment will vary depending upon the particular factors of each case, including the type of neurodegeneration, the stage of the neurodegeneration, the subject's weight, the severity of the subject's condition and the method of administration. This amount can be readily determined by the skilled artisan. In one embodiment, the present invention provides a method for treating or preventing neurodegeneration in a subject, comprising administering a compound having the general formula: 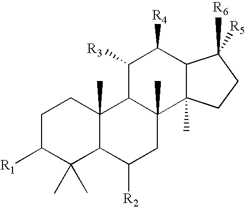
where R₁ is selected from the group consisting of α-OH, β-OH, α-O—X, β-O—X, α-R₇COO—, β-R₇COO—, α-R₇PO₃—, β-R₇PO₃—, α-NR₈R₉, β-NR₈R₉, ═O(oxo), ═NOH, ═NC(O)NHNH₂ and CH₂—X; where X is an alkyl or a carbohydrate containing one or more sugars or acylated derivatives thereof; R₇ is H, OH, an amino group, an alkenyl, aryl, or alkyl; R₈ is H, alkyl, aryl, acyl, or SO₂NHR₁₀; R₉ is H, alkyl, aryl, acyl, or SO₂NHR₁₀; R₁₀ is NH2, OH, alkyl, aryl, or cycloalkyl; R₂ is selected from the group consisting of H, OH, OAc and O—X, where X is a carbohydrate containing one or more sugars or acylated derivatives thereof, Ac═CH₃CO or acyl; R₃ is selected from the group consisting of H, OH, O—X and OAc, where X is an alkyl or a carbohydrate containing one or more sugars or acylated derivatives thereof; R₄ is selected from the group consisting of H, OH, O—X and OAc, where X is an alkyl or a carbohydrate containing one or more sugars or acylated derivatives thereof; R5 is H or OH; and R6 is alkenyl, aryl, or alkyl.

In one embodiment of the invention, Alzheimer's disease is treated in a subject in need of treatment by administering to the subject a therapeutically effective amount of a dammarane composition, a dammarane or analogue or homologue thereof effective to treat the Alzheimer's disease. The subject is preferably a mammal (e.g., humans, domestic animals and commercial animals, including cows, dogs, monkeys, mice, pigs and rats), and is most preferably a human. The term analogue as used in the present invention refers to a chemical compound that is structurally similar to another and may be theoretically derivable from it, but differs slightly in composition. For example, an analogue of the dammarane (20S)Rg3 is a compound that differs slightly from (20S)Rg3 (e.g., as in the replacement of one atom by an atom of a different element or in the presence of a particular functional group), and may be derivable from (20S)Rg3. The term homologue as used in the present invention refers to members of a series of compounds in which each member differs from the next member by a constant chemical unit. The term synthesize as used in the present invention refers to formation of a particular chemical compound from its constituent parts using synthesis processes known in the art. Such synthesis processes include, for example, the use of light, heat, chemical, enzymatic or other means to form particular chemical composition.

The terms “therapeutically effective amount” or “effective amount,” as used herein, mean the quantity of the composition according to the invention which is necessary to prevent, cure, ameliorate or at least minimize the clinical impairment, symptoms or complications associated with Alzheimer's disease in either a single or multiple dose. The amount of dammarane effective to treat Alzheimer's disease will vary depending on the particular factors of each case, including the stage or severity of Alzheimer's disease, the subject's weight, the subject's condition and the method of administration. The skilled artisan can readily determine these amounts. For example, the clinical impairment or symptoms of Alzheimer's disease may be ameliorated or minimized by diminishing any dementia or other discomfort suffered by the subject; by extending the survival of the subject beyond that which would otherwise be expected in the absence of such treatment; or by inhibiting or preventing the progression of the Alzheimer's disease.

Treating Alzheimer's disease, as used herein, refers to treating any one or more of the conditions underlying Alzheimer's disease including, without limitation, neurodegeneration, senile plaques, neurofibrillary tangles, neurotransmitter deficits, dementia, and senility. As used herein, preventing Alzheimer's disease includes preventing the initiation of Alzheimer's disease, delaying the initiation of Alzheimer's disease, preventing the progression or advancement of Alzheimer's disease, slowing the progression or advancement of Alzheimer's disease, and delaying the progression or advancement of Alzheimer's disease.

Prior to the present invention, the effect of dammaranes and dammaranes on production of beta amyloid protein was unknown. The present invention establishes that dammaranes such as those disclosed herein or their analogues or homologues can also be used to prevent and treat Alzheimer's disease patients. This new therapy provides a unique strategy to treat and prevent neurodegeneration and dementia associated with Alzheimer's disease by modulating the production of Aβ42. Further, neurodegeneration and dementias not associated with Alzheimer's disease can also be treated or prevented using the dammaranes of the present invention to modulate the production of Aβ42.

The dammaranes of the present invention include natural or synthetic functional variants, which have dammarane biological activity, as well as fragments of dammarane having dammarane biological activity. As further used herein, the term “dammarane biological activity” refers to activity that modulates the generation of the highly amyloidogenic Aβ42, the 42-amino acid isoform of amyloid β-peptide. In an embodiment of the invention, the dammarane reduces the generation of Aβ42 in the cells of a subject.

Methods of preparing dammaranes such as Rk1, (20S)Rg3 and Rg5, as well as their analogues and homologues, are well known in the art. For example, U.S. Pat. No. 5,776,460, the disclosure of which is incorporated herein in its entirety, describes preparing a processed ginseng product in which a ratio of dammarane (Rg3+Rg5) to (Rc+Rd+Rb1+Rb2) is above 1.0. The processed product disclosed in U.S. Pat. No. 5,776,460 is prepared by heat-treating ginseng at a high temperature of 120° to 180° C. for 0.5 to 20 hours. The dammaranes of the present invention may be isolated dammarane compounds or isolated and further synthesized dammarane compounds. The isolated dammaranes of the present invention can be further synthesized using processes including, but not necessarily limited to, heat, light, chemical, enzymatic or other synthesis processes generally known to the skilled artisan.

In a method of the present invention, the dammarane compound is administered to a subject in combination with one or more different dammarane compounds. Administration of a dammarane compound “in combination with” one or more different dammarane compounds refers to co-administration of the therapeutic agents. Co-administration may occur concurrently, sequentially, or alternately. Concurrent co-administration refers to administration of the different dammarane compounds at essentially the same time. For concurrent co-administration, the courses of treatment with the two or more different dammaranes may be run simultaneously. For example, a single, combined formulation, containing both an amount of a particular dammarane compound and an amount of a second different dammarane compound in physical association with one another, may be administered to the subject. The single, combined formulation may consist of an oral formulation, containing amounts of both dammarane compounds, which may be orally administered to the subject, or a liquid mixture, containing amounts of both the dammarane compounds, which may be injected into the subject.

It is also within the confines of the present invention that an amount of one particular dammarane compound and an amount one or more different dammarane compound may be administered concurrently to a subject, in separate, individual formulations. Accordingly, the method of the present invention is not limited to concurrent co-administration of the different dammarane compounds in physical association with one another.

In the method of the present invention, the dammarane compounds also may be co-administered to a subject in separate, individual formulations that are spaced out over a period of time, so as to obtain the maximum efficacy of the combination. Administration of each therapeutic agent may range in duration from a brief, rapid administration to a continuous perfusion. When spaced out over a period of time, co-administration of the dammarane compounds may be sequential or alternate. For sequential co-administration, one of the therapeutic agents is separately administered, followed by the other. For example, a full course of treatment with 3-[3-(3,4-dihydroxyphenyl)-2-propenoate]-(3β,20S)-Dammar-24-ene-3,20-diol derivative may be completed, and then may be followed by a full course of treatment with a Dammare-23-ene-3α,20(S) derivative. Alternatively, for sequential co-administration, a full course of treatment with Dammare-23-ene-3α,20(S) derivative may be completed, then followed by a full course of treatment with a 3-[3-(3,4-dihydroxyphenyl)-2-propenoate]-(3β,20S)-Dammar-24-ene-3,20-diol derivative. For alternate co-administration, partial courses of treatment with the Dammare-23-ene-3α, 20(S) derivative may be alternated with partial courses of treatment with the 3-[3-(3,4-dihydroxyphenyl)-2-propenoate]-(3β,20S)-Dammar-24-ene-3,20-diol derivative, until a full treatment of each therapeutic agent has been administered.

The therapeutic agents of the present invention (i.e., the dammarane and analogues and analogues thereof) may be administered to a human or animal subject by known procedures including, but not limited to, oral administration, parenteral administration (e.g., intramuscular, intraperitoneal, intravascular, intravenous, or subcutaneous administration) and transdermal administration. Preferably, the therapeutic agents of the present invention are administered orally or intravenously.

For oral administration, the formulations of the dammarane may be presented as capsules, tablets, powders, granules, or as a suspension. The formulations may have conventional additives, such as lactose, mannitol, cornstarch or potato starch. The formulations also may be presented with binders, such as crystalline cellulose, cellulose analogues, acacia, cornstarch, or gelatins. Additionally, the formulations may be presented with disintegrators, such as cornstarch, potato starch or sodium carboxymethyl cellulose. The formulations also may be presented with dibasic calcium phosphate anhydrous or sodium starch glycolate. Finally, the formulations may be presented with lubricants, such as talc or magnesium stearate.

For parenteral administration, the formulations of the dammarane may be combined with a sterile aqueous solution which is preferably isotonic with the blood of the subject. Such formulations may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulations may be presented in unit or multi-dose containers, such as sealed ampules or vials. Moreover, the formulations may be delivered by any mode of injection including, without limitation, epifascial, intracapsular, intracutaneous, intramuscular, intraorbital, intraperitoneal (particularly in the case of localized regional therapies), intraspinal, intrasternal, intravascular, intravenous, parenchymatous or subcutaneous.

For transdermal administration, the formulations of the dammarane may be combined with skin penetration enhancers, such as propylene glycol, polyethylene glycol, isopropanol, ethanol, oleic acid, N-methylpyrrolidone, and the like, which increase the permeability of the skin to the therapeutic agent, and permit the therapeutic agent to penetrate through the skin and into the bloodstream. The therapeutic agent/enhancer compositions also may be further combined with a polymeric substance, such as ethylcellulose, hydroxypropyl cellulose, ethylene/vinylacetate, polyvinyl pyrrolidone, and the like, to provide the composition in gel form, which may be dissolved in a solvent such as methylene chloride, evaporated to the desired viscosity, and then applied to backing material to provide a patch.

The dose of the dammarane of the present invention may also be released or delivered from an osmotic mini-pump. The release rate from an elementary osmotic mini-pump may be modulated with a microporous, fast-response gel disposed in the release orifice. An osmotic mini-pump would be useful for controlling release, or targeting delivery, of the therapeutic agents.

It is within the confines of the present invention that the formulations of the dammarane may be further associated with a pharmaceutically acceptable carrier, thereby comprising a pharmaceutical composition. The pharmaceutically acceptable carrier must be “acceptable” in the sense of being compatible with the other ingredients of the composition, and not deleterious to the recipient thereof. Examples of acceptable pharmaceutical carriers include, but are not limited to, carboxymethyl cellulose, crystalline cellulose, glycerin, gum arabic, lactose, magnesium stearate, methyl cellulose, powders, saline, sodium alginate, sucrose, starch, talc, and water, among others. Formulations of the pharmaceutical composition may conveniently be presented in unit dosage.

The formulations of the present invention may be prepared by methods well known in the pharmaceutical art. For example, the active compound may be brought into association with a carrier or diluent, as a suspension or solution. Optionally, one or more accessory ingredients (e.g., buffers, flavoring agents, surface active agents, and the like) also may be added. The choice of carrier will depend upon the route of administration. The pharmaceutical composition would be useful for administering the therapeutic agents of the present invention (i.e., dammaranes their analogues and analogues, either in separate, individual formulations, or in a single, combined formulation) to a subject to treat Alzheimer's disease. The therapeutic agents are provided in amounts that are effective to treat or prevent Alzheimer's disease in the subject. These amounts may be readily determined by the skilled artisan.

The effective therapeutic amounts of the dammarane will vary depending on the particular factors of each case, including the stage of the Alzheimer's disease, the subject's weight, the severity of the subject's condition, and the method of administration. For example, 3-[3-(3,4-dihydroxyphenyl)-2-propenoate]-(3β,20S)-Dammar-24-ene-3,20-diol can be administered in a dosage of about 5 μg/day to 1500 mg/day. Preferably, 3-[3-(3,4-dihydroxyphenyl)-2-propenoate]-(3β,20S)-Dammar-24-ene-3,20-diol is administered in a dosage of about 1 mg/day to 1000 mg/day. The appropriate effective therapeutic amounts of any particular dammarane compound within the listed ranges can be readily determined by the skilled artisan depending on the particular factors of each case.

The present invention additionally encompasses methods for preventing Alzheimer's disease in a subject with a pre-Alzheimer's disease condition, comprising administering to the subject a therapeutically effective amount of a dammarane compound. As used herein, “pre-Alzheimer's disease condition” refers to a condition prior to Alzheimer's disease. The subject with a pre-Alzheimer's disease condition has not been diagnosed as having Alzheimer's disease, but nevertheless may exhibit some of the typical symptoms of Alzheimer's disease and/or have a medical history likely to increase the subject's risk to developing Alzheimer's disease.

The invention further provides methods for treating or preventing Alzheimer's disease in a subject, comprising administering to the subject a therapeutically effective amount of dammarane compound.

### EXAMPLES

The following examples illustrate the present invention, which are set forth to aid in the understanding of the invention, and should not be construed to limit in any way the scope of the invention as defined in the claims which follow thereafter.

The inventors have unexpectedly found that particular Dammarane compounds lower the production of Aβ42 in cells, thus treating AD and non-AD associated neuropathogenesis and/or preventing the progression of AD and non-AD associated neuropathogenesis.

### Example 1

The genuine sapogenines of the ginseng glycosides are structurally similar to some chemical constituents of other plants. Betulafolienetriol [dammar-24-ene-3α,12β,20(S)-triol}] isolated from birch leaves differ from the genuine sapogenin of ginseng glycosides, 20(S)-protopanaxadiol, in the configuration at C-3 only. Therefore, betulafolienetriol, cheap and relatively accesable, makes a desirable sustrate to prepare 20(S)-protopanaxadiol and its glycoside Rg3, Rg5, and Rk1.

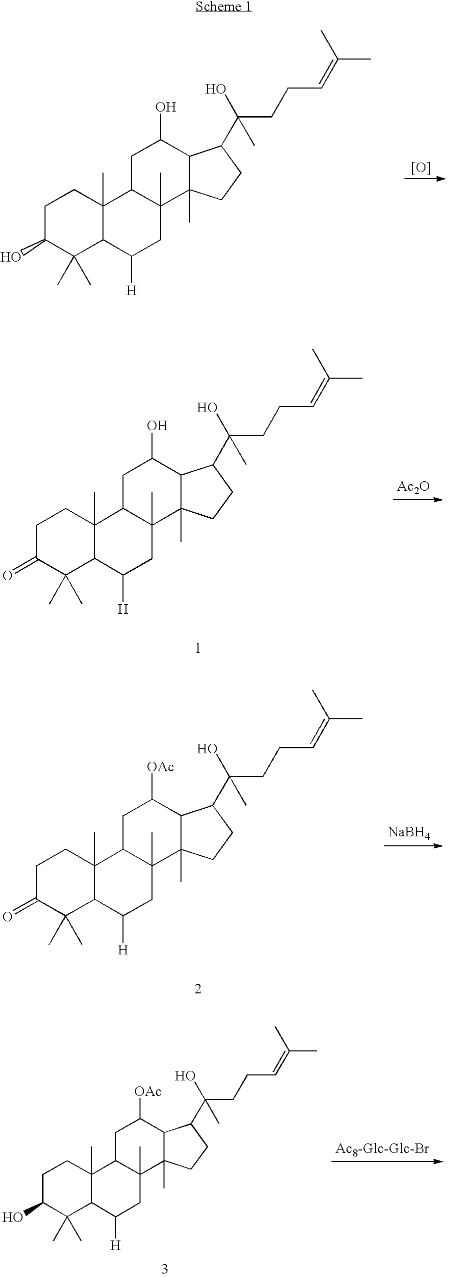

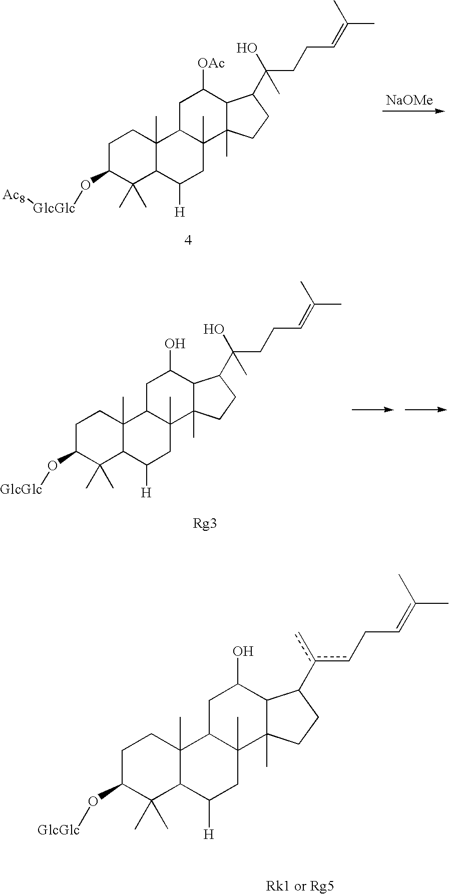

Betulafolienetriol was isolated from an ethereal extract of the leaves Btula pendula, followed by chromatography on silica gel and crystallization from acetone: mp 195-195°, lit. 197-198° (Fischer et al. (1959) Justus Liebigs Ann. Chem. 626:185).

The 12-O-acetyl derivative of 20(S)-protopanaxadiol (3) is prepared from betulafolienetriol by the sequence of reactions shown in Scheme 1. Betulafolienetriol is oxidized to ketone 1, dammar-24-ene-12β,20(S)-diol-3-one, mp 197-199°, lit 196-199°, (yield: 60%), which is acetylated with acetic anhydride in pyridine to give compound 2,12-O-Acetyl-dammar-24-ene-12β,20(S)-diol-3-one (yield: 100%?) (Nagal, et al., (1973) Chem. Pharm. Bull. 9:2061). ¹H NMR (CDCl3) of the compound 2: 0.90 (s, 3H), 0.95 (s, 3H), 1.0 (s, 6H), 1.1 (s, 3H), 1.1 (s, 3H), 1.65 (s, 3H), 1.72 (s, 3H), 2.1 (s, 3H), 3.04 (s, 1H), 4.73 (td, 1H), 5.17 (t, 1H). Sodium borohydride reduction of the compound 2 in 2-propanol affords compound 3,12-O-Acetyl-dammar-24-ene-3β,12β,20(S)-triol (yield: 90%). ¹H NMR (CDCl₃) of the compound 3: 0.78 (s, 3H), 0.86 (8, 3H), 0.95 (s, 3H), 1.0 (s, 3H), 1.02 (s, 3H), 1.13 (s, 3H), 1.64 (s, 3H), 1.71 (s, 3H), 2.05 (s, 3H, OAc), 3.20 (dd, 1H, H-3α), 4.73 (td, 1H, H-12α), 5.16 (t, 1H, H-24).

Condensation of compound 3 with O-acetylate-sugar bromide in the presence of silver oxide and molecular sieves 4A in dichloroethane results in formation of compound 4 (yield: 50%). Specifically, a mixture of compound 3 (1.08 g, 2 mmol), silver oxide (1.4 g, 6 mmol), α-acetobromoglucose (2.47 g, 6 mmol), molecular sieves 4A (1.0 g) and dichloroethane (20 ml) was agitated at ambient temperature until the acetobromoglucose had reacted (TLC). The reaction mixture was then diluted with CHCl₃ and filtered. The solvent was evaporated and the residue was washed with hot water to remove the excess of glucose derivatives. Silica gel column chromatography (8:1 n-hexane-acetone) gave compound 4 (853 mg). Deprotection of the glucoside 4 gives dammarane Rg3 which is concerted to Rk1 or Rg5 in 2 steps.

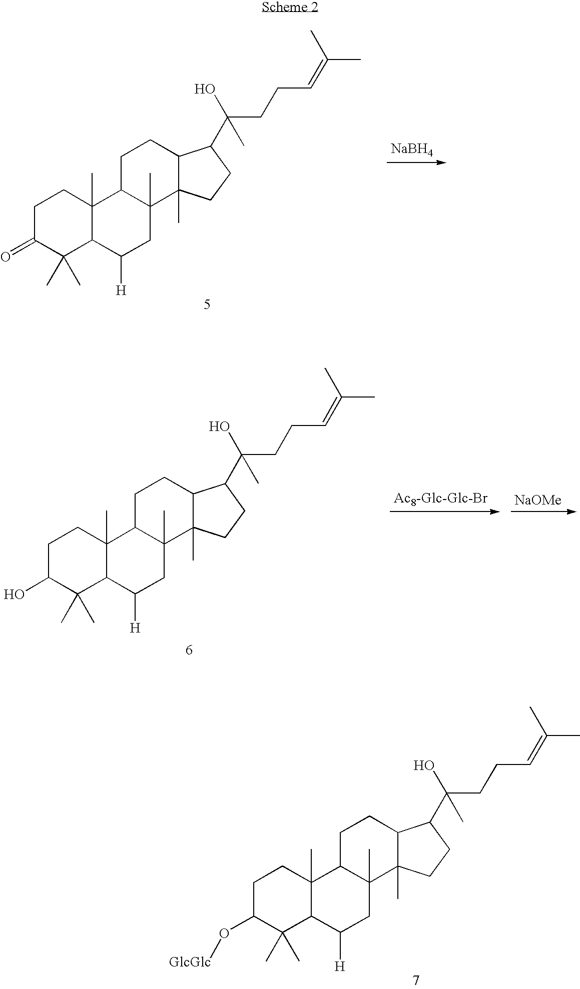

### Example 2

### Synthesis of Compounds 2-14 from Dipterocarpole

Dipterocarpole, the major component of Dammar resin, can be reduced with reducing agents to provide two types of alcohols 2a (3α) and 2b (3β). The reduction of Dipterocarpole with LiBH(sec-Bu)₃ yields 3α and 3β as a mixture with a ratio dependent on the reaction temperature. Higher temperature yields more of the 3a isomer. The reduction with NaBH₄ yields 2b as a major product. Compounds 2a and 2b can be separated by silica gel chromatography. Compounds 2a and 2b are used, separately or as a mixture, for the synthesis of compounds 3-9 as shown in Scheme 1, 2, 3.

Compounds 3a and 3b are synthesized by esterification of 2a and 2b with di-O-acetylcaffeoyl chloride, followed by hydrolysis of the acetate with NaHCO₃ (Scheme 1).

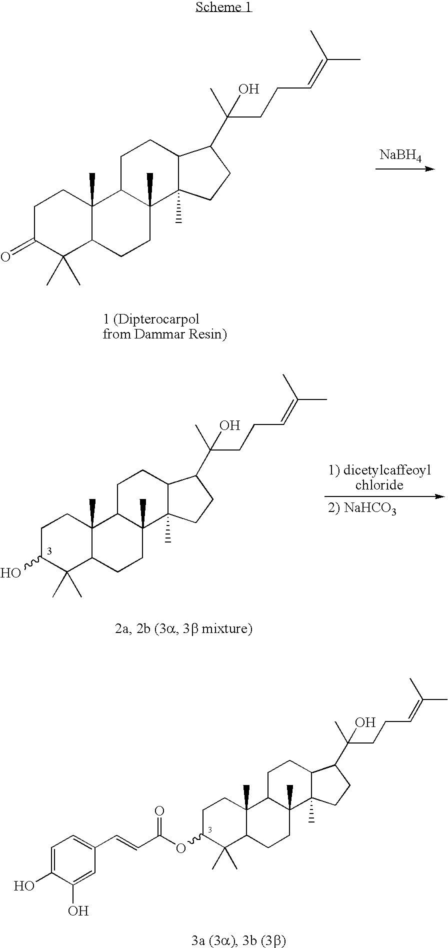

Compounds 4a and 4b are obtained by oxidation of 2a and 2b with m-chloroperbenzoic acid (MCPBA). Selective esterification of 4a, 4b with acetic anhydride in pyridine provide 5a and 5b. Similarly, 2a and 2b are selectively esterified with acetic anhydride in pyridine to produces 3-acetate of 2a and 2b (Scheme 2).

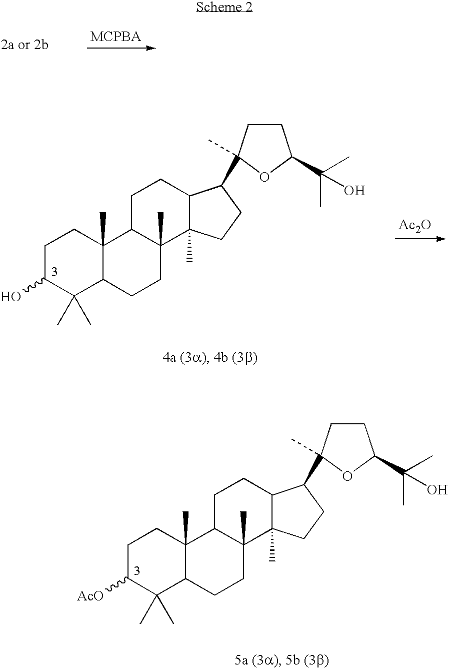

Introduction of a sugar side chain to the hydroxyl group of the compounds described above is achieved by glycosation with the appropriate bromonated sugar. For example, the reaction of 2a and 2b with Ac8-Glc-Glc-Br in the presence of Ag₂O, followed by deprotection of the acetyl groups gives compounds 6a and 6b (Scheme 3). Phosphoric acid containing 7a and 7b are synthesized by reaction of 2a and 2b with dibenzylphosphoric chloride in the presence of a base, followed by hydrogenation with H₂/Pd—C (Scheme 3).

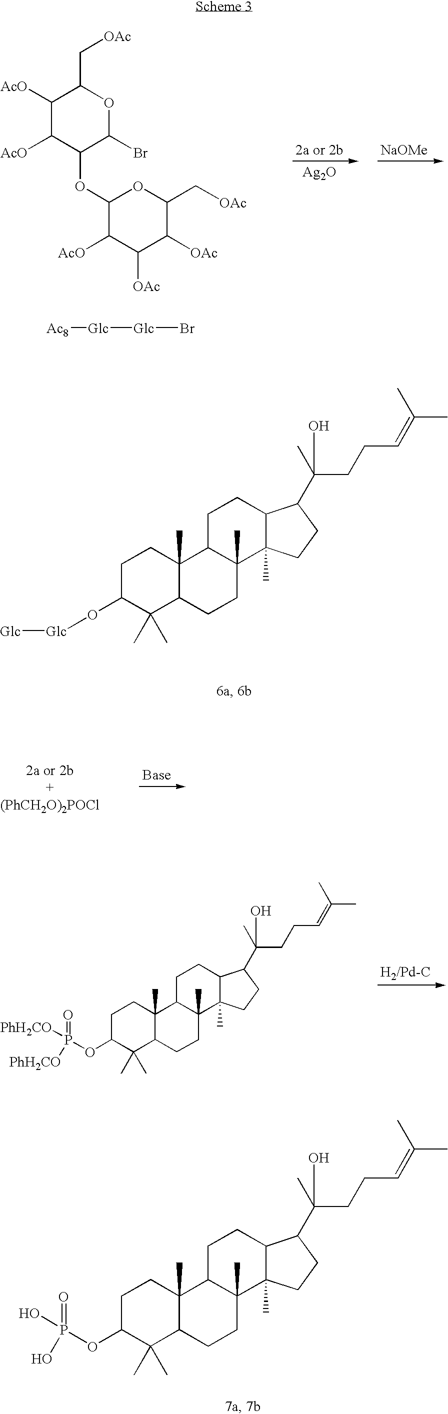

The reaction of dipterocarpole with NH₂OH in ethanol yields an oxime that is reduced with sodium to amine 8. Similarly, the reaction of dipterocarpole with NH₂CONHNH₂ in ethanol yields the corresponding semicarbazone. Reaction of the amino group of 8 with a variety of acid anhydride or sulfonyl chloride provides amides or sulfonamide. As an example, treatment of 8 with diglycolic anhydride in the presence of base provides compounds 9 (Scheme 4). The 20-hydroxy group of the compounds can be removed to form a double bond by dehydroxylation in DMSO at 120° C. The C-20 and C-24 double bonds are hydrogenated using H₂/Pd—C to provide alkyl side chain. The C20 double bond is converted to a ketone with O₃/Ph₃P which is used for synthesis of other analogs with different side chains.

Dammar-24-ene-3α,20(S)-diol (2a): Commercially available dipterocarpole (44 mg) in THF (3 ml) was added LiBH(sec-Bu)₃ (0.3 ml 1M in THF). The reaction mixture was heated to 50 C and stirred at 50 C for 2 h. The reaction was quenched by NH₄Cl carefully and was added ethyl acetate (10 ml). The organic phase was dried over Na₂SO4 and removal of the solvents provided the crude product which was purified by chromatography over SiO₂ (petroleum ether/ethyl acetate=10/1).

Dammar-24-ene-3β,20(S)-diol (2b): Commercially available Dammar resin (100 g) was dissolved in iso-propanol (400 ml). After filtration of insoluble matters, the solution was added sodium borohydride (NaBH4) (20 g). TLC showed a mixture of at least 5 spots, but the major spot is the desired product 2b. The reaction mixture was stirred for 4 h. at room temperature, and then quenched with sat. NH₄Cl solution. The solvents were removed by evaporation and the residue was dissolved in ethyl acetate (150 ml) and washed with H₂O. The organic phase was dried over Na₂SO4. The pure product 2b was obtained by purification with chromatography (petroleum ether/ethyl acetate=10/1) or crystallization.

20,24-epoxy-(3α,24R)-Dammarane-3,25-diol (4a): Dammar-24-ene-3α, 20(S)-diol (2a) (70 mg) in CH₂Cl₂ (5 ml) was added t-butylhydroperoxide (0.1 ml 5M in CH₂Cl₂) and Vo(acac)₂ (1.2 mg). The reaction mixture was stirred for 2 h. The product 4a was purified by chromatography (petroleum ether/ethyl acetate=10/1). Similarly, the following compounds were synthesized: 20,24-epoxy-(3□,24R)-Dammarane-3,25-diol (4b).

Condensation of 2a or 2b with a-acetobromoglucose in the presence of silver oxide. A mixture of compound 2a (444 mg, 1.0 mmol), silver oxide (696 mg g, 3 mmol), α-acetobromoglucose (1.23 g, 3 mmol), molecular sieves 4 A (1.0 g) and dichloromethane (20 ml) was stirred at r. t. until the acetobromoglucose had reacted (TLC). The reaction mixture was then diluted with CHCl₃ and filtered. The solvent was evaporated and the residue was washed with hot water to remove an excess of glucose derivatives. Silica gel column chromatography. (petroleum ether/ethyl acetate=10/1) yielded tetra-acetated (3(X)-20-hydroxydammar-24-ene-3-yl-glucopyranoside. The tetraacetate was removed by reaction with NaOMe in methanol. Similarly the following compounds were synthesized: 6a, 6b and (12,8)-20-hydroxydammar-24-ene-3, yl-D-Glc (1-2)glucopyranoside, (24R)-20,24-epoxy-25-hydroxydammaran-3-yl-β-D-Glucopyranoside.

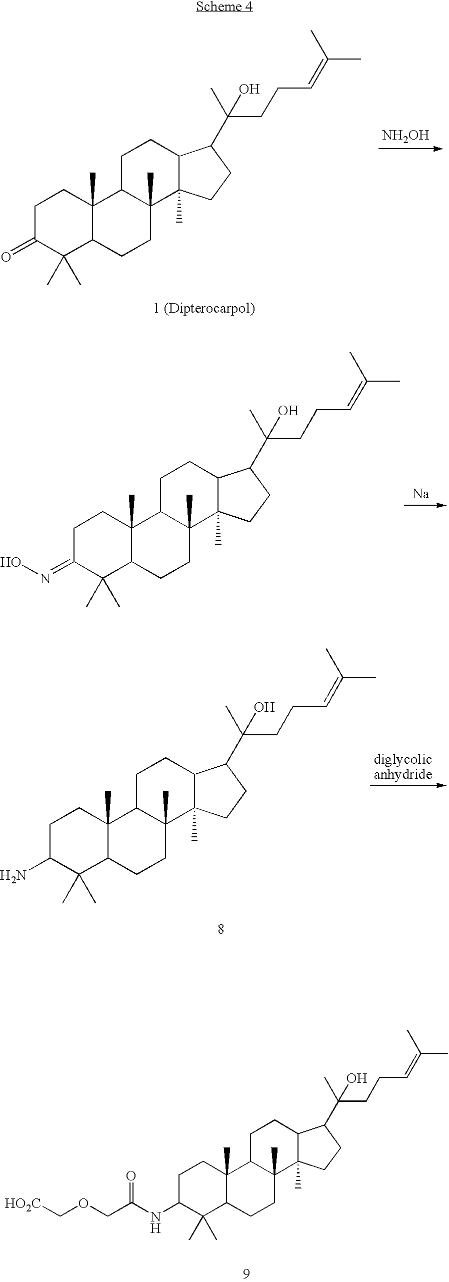

### Example 3

### Synthesis of Compounds Rg3, Rg5, and Rk1 and Compounds 11-18 from Betulafolienetriol (10)

Betulafolienetriol [dammar-24-ene-3α,12β,20(S)-triol] (10) isolated from birch leaves differs from 20(S)-protopanaxatriol from ginseng only in the configuration at C-3. For this reason, we chose Betulafolienetriol as a relatively accessible starting material to prepare 20(S)-protopanaxadiol, its glycosides Rg3, Rg5, and Rk1 and Compounds 11-18.

The 12-O-acetyl derivative of 20(S)-protopanaxadiol (13) is prepared from betulafolienetriol (10) by the sequence of reactions shown in Scheme 5. Betulafolienetriol is oxidized to ketone 11 in 60% yield and then acetylated with acetic anhydride in pyridine to give 3-keto-12-O-acetyl derivative 12 quantitatively. Sodium borohydride reduction of 12 in 2-propanol affords 12-O-acetate 13 in 90% yield. Condensation of compound 13 with Ac8-Glc-Glc-Br in the presence of silver oxide and molecular sieves in CH₂H₂ provides compound 14 in 50% yields. Deprotection of the acetylated glucoside 14 with NaOMe provides dammarane Rg3 which is converted to Rk1 and Rg5 as a mixture with H₂SO₄ in DMSO.

Betulafolienetriol (10) was isolated from an ethereal extract of the leaves Betula pendula, followed by silica gel chromatography and crystallization from acetone: mp 195-196°, lit. 197-1980 [Fischer, et. al., Justus Liebigs Ann. Chem., 626 (1959) 185].

Dammar-24-ene-12β,20(S)-diol-3-one (11) was obtained by oxidation of Betulafolienetriol with chromic anhydride in pyridine in the yield of 60%. mp 197-199°. lit. 196-1990 (Nagai, et. al., Chem., Pharm. Bull., 9 (1973) 2061).

12-O-Acetyl-dammar-24-ene-12β,20(S)-diol-3-one (12) was obtained by conventional acetylation of 11 with Ac₂O in pyridine. ¹H NMR (CDCl₃): 0.90 (s, 3H), 0.95 (s, 3H), 1.0 (s, 6H), 1.1 (s, 3H), 1.1 (s, 3H), 1.65 (s, 3H), 1.72 (s, 3H), 2.1 (s, 3H), 3.04 (s, 1H), 4.73 (td, 1H), 5.17 (t, 1H).

12-O-Acetyl-dammar-24-ene-3β,12β,20(S)-triol (13) was obtained by reducing 12 with NaBH₄ in 2-propanol at 0° C. in the yield of 90%. ¹H NMR (CDCl₃): 0.78 (s, 3H), 0.86 (8, 3H), 0.95 (s, 3H), 1.0 (s, 3H), 1.02 (s, 3H), 1.13 (s, 3H), 1.64 (s, 3H). 1.71 (s, 3H), 2.05 (s, 3H, OAc), 3.20 (dd, 1H, H-3a), 4.73 (td, 1H, H-12a), 5.16 (t, 1H, H-24).

Condensation of 13 with a-acetobromoglucose in the presence of silver oxide. A mixture of the 12-O-acetate 13 (1.08 g, 2 mmol), silver oxide (1.4 g, 6 mmol), α-5 acetobromoglucose (2.47 g, 6 mmol), molecular sieves 4 A (1.0 g) and dichloroethane (20 ml) was agitated at r. t. until the acetobromoglucose had reacted (TLC). The reaction mixture was then diluted with CHCl3 and filtered. The solvent was evaporated and the residue was washed with hot water to remove an excess of glucose derivatives. Silica gel column chromatography. (8:1 n-hexane-acetone) yielded 14 (853 mg. 50%).

Rg3 was obtained by deacetylation of compound 14 using methanolic 1 M NaOMe.

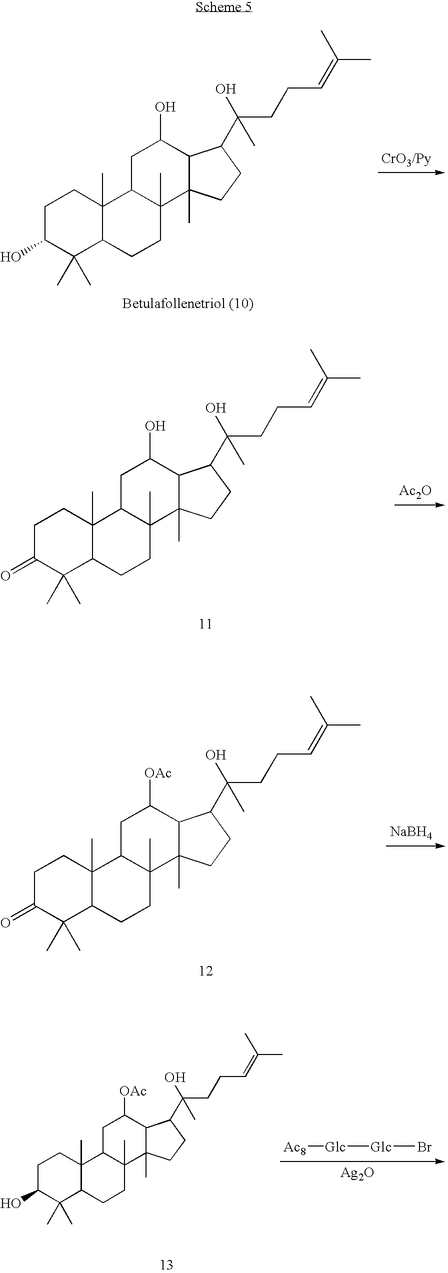

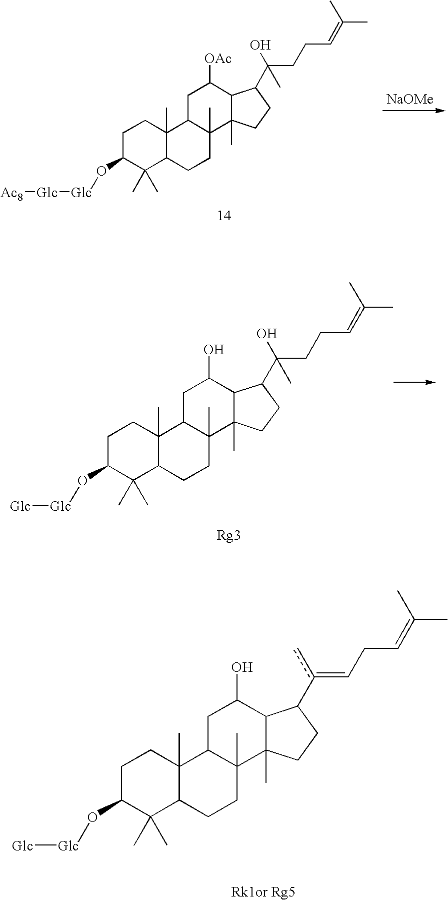

Betulafolienetriol [dammar-24-ene-3a,12{3,20(S)-triol] (10) is also converted to 15, 16, 17, 18 as shown in Scheme 6 by selective esterification (Scheme 6). Using methods similar to those used in the synthesis of 6 and 7 as shown in Scheme 3, a sugar side chain or phosphoric acid is introduced to the 3α-hydroxy group of Betulafolienetriol to form glycoside 19 and phosphoric analog 20, respectively.

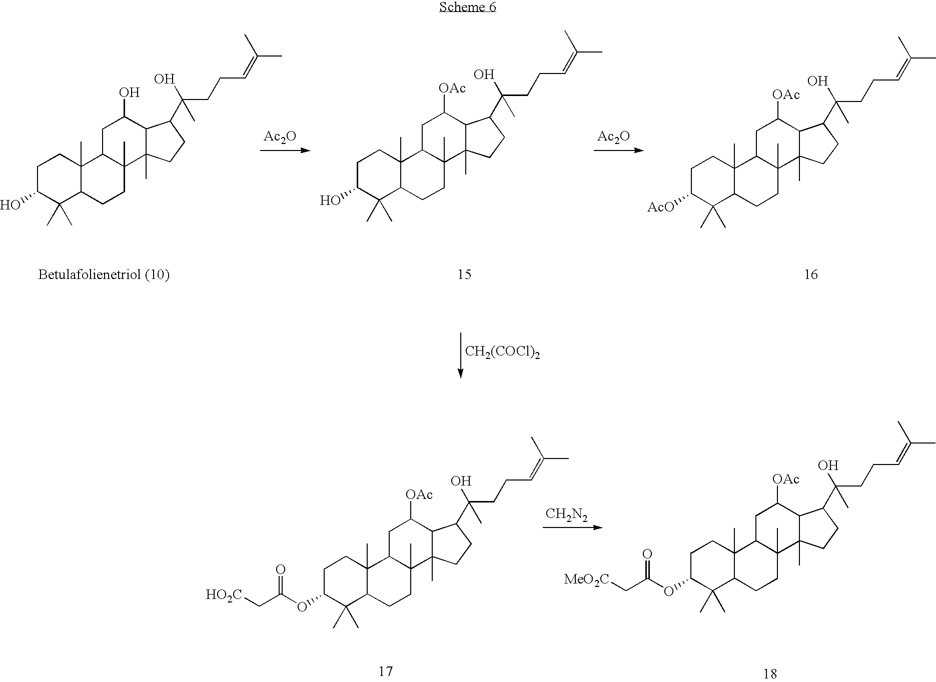

Using methods similar to those used in the synthesis of 4 and 5 as shown in Scheme 2, Betulafolienetriol or its 3β-isomer is oxidized to epoxy compounds 21a and 21b which react with various acid anhydride or acid chloride to provide 3-acylated compounds such as 22, 23 and 24 (Scheme 7).

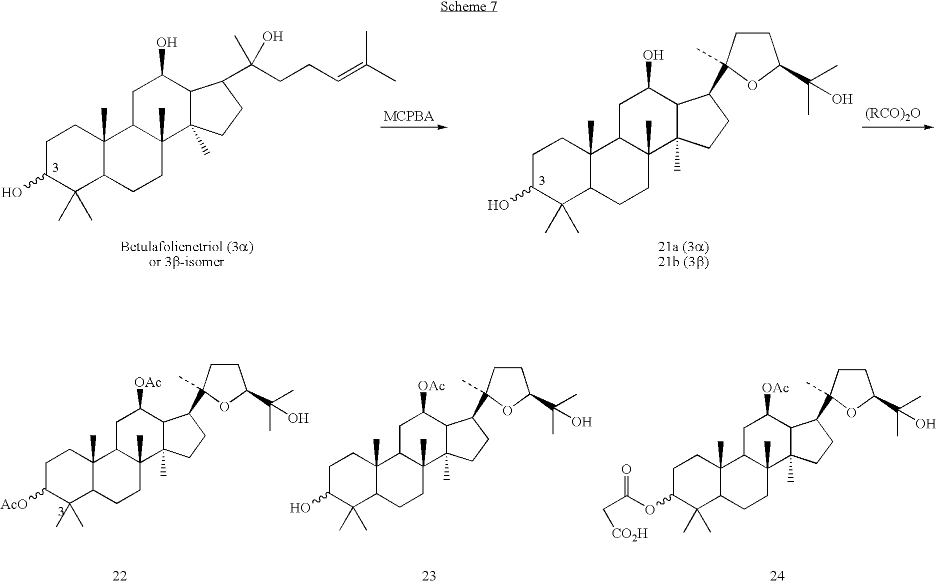

### Example 4

### Preparation of the Compounds by Extract from Plants Such as Birch

In addition to the compounds obtained by synthesis described above, we obtained some of compounds of structure I from commercial source who extract the compounds from birch tree.

### Example 5

### Biological Activity Assay

CHO cells stably transfected with human APP751 (CHO-APP cells) were treated with compounds as described above. The compounds were used at 50 μg/ml for 6 and 24 hours. Levels of secreted Aβ40 and Aβ42 were determined using a commercial Aβ ELISA kit (Biosource) and normalized to cell-associated full-length APP. The relative levels of Aβ40 (white bar) and Aβ42 (black bar) were normalized to values obtained from vehicle-treated cells and are shown as % to control. The results are shown in FIG. 20 as examples. Compound D5, D6, D10, D11, D12, D15 exhibited Aβ-lowering activities. In contrast, some of the compounds selectively potentiated Aβ42 production (e.g., D1, D2, D3, D7 and D9). (D10=20(S), 24(R)-epoxydammarane-3β,25-diol; D2=Dammar-24-ene-3β,20(S)-diol; D3=reduction mixture of dammar resin by NaBH₄; D5=Dammar resin mixture; D6=dipterocarpol; D7=t-butylhydroperoxide-oxidized products of D3. D8=3-acetyl Dammar-24-ene-3A, 20(S)-diol; D9=3-acetyl 20(S),24(R)-epoxydammarane-3β,25-diol; D10=20(S),24(R)-epoxydammarane-3×, 25-diol; D11=Dammar-24-ene-3×, 20(S)-diol; D12=tetraacetyl 20-hydroxydammar-24-ene-3-yl-glucopyranoside; D13=20-hydroxydammar-24-ene-3-yl-glucopyranoside; D15=20(S), 24(R)-epoxydammarane-3-oxo, 25-ol.

While the foregoing invention has been described in some detail for purposes of clarity and understanding, it will be appreciated by one skilled in the art, from a reading of the disclosure, that various changes in form and detail can be made without departing from the true scope of the invention in the appended claims.

### Example 6

The benefits of dammarane therapy for treating AD associated neurodegeneration can be demonstrated in a murine model of AD. Specifically, the dammarane compounds D5, D6 and D10 can be used to treat mice suffering from AD associated neurodegeneration.

Mice expressing human APP as well as mice expressing the Swedish familial Alzheimer's disease mutant form of APP can be obtained from the Jackson Laboratory, 600 Main Street, Bar Harbor, Me. 04609. Four groups of mice can then be studied: (1) APP mice without dammarane treatment (placebo); (2) Swedish mice without dammarane treatment (placebo); (3) APP mice+D5 (100 μg/l/day); and (4) Swedish mice+D10 (100 μg/μl/day). After approximately 16 weeks of injection therapy, amounts of Aβ42 in the serum of the mice can be measured. It is expected that the results of this study will demonstrate the general benefits of dammarane therapy for treating AD associated neuordegeneration. APP and Swedish mice without dammarane treatment should have significantly higher levels of serum Aβ42 and demonstrate behavior characterisitic of neurodegeneration, as compared with APP and Swedish mice receiving dammarane treatment.

While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A compound having the general formula: 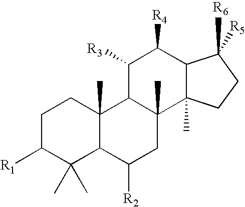
where R₁ is selected from the group consisting of α-OH, β-OH, α-O—X, β-O—X, α-R₇COO—, β-R₇COO—, α-R₇PO₃—, β-R₇PO₃—, α-NR₈R₉, β-NR₈R₉, ═O(oxo), ═NOH, ═NC(O)NHN—H₂ and CH₂—X; where X is an alkyl or a carbohydrate containing one or more sugars or acylated derivatives thereof; R₇ is H, OH, an amino group, an alkenyl, aryl, or alkyl; R₈ is H, alkyl, aryl, acyl, or SO₂NHR₁₀; R₉ is H, alkyl, aryl, acyl, or SO₂NHR₁₀; R₁₀ is NH2, OH, alkyl, aryl, or cycloalkyl;
R₂ is selected from the group consisting of H, OH, OAc and O—X, where X is a carbohydrate containing one or more sugars or acylated derivatives thereof, Ac═CH₃CO or acyl;
R₃ is selected from the group consisting of H, OH, O—X and OAc, where X is an alkyl or a carbohydrate containing one or more sugars or acylated derivatives thereof;
R₄ is selected from the group consisting of H, OH, O—X and OAc, where X is an alkyl or a carbohydrate containing one or more sugars or acylated derivatives thereof; R5 is H or OH; and
R6 is alkenyl, aryl, or alkyl.

2. The compound of claim 1, where R₇ further contains oxygen, nitrogen or phosphorus.

3. The compound of claim 1, where R₆ further contains a functional group selected from the group consisting of hydroxyl, ether, ketone, oxime, hydrazone, imine and Schiff base.

4. The compound of claim 1, where the sugar is selected from the group consisting of Glc, Ara(pyr), Ara(fur), Rha and Xyl and acylated derivatives thereof.

5. The compound of claim 1, where R₆ is selected from the group consisting of: 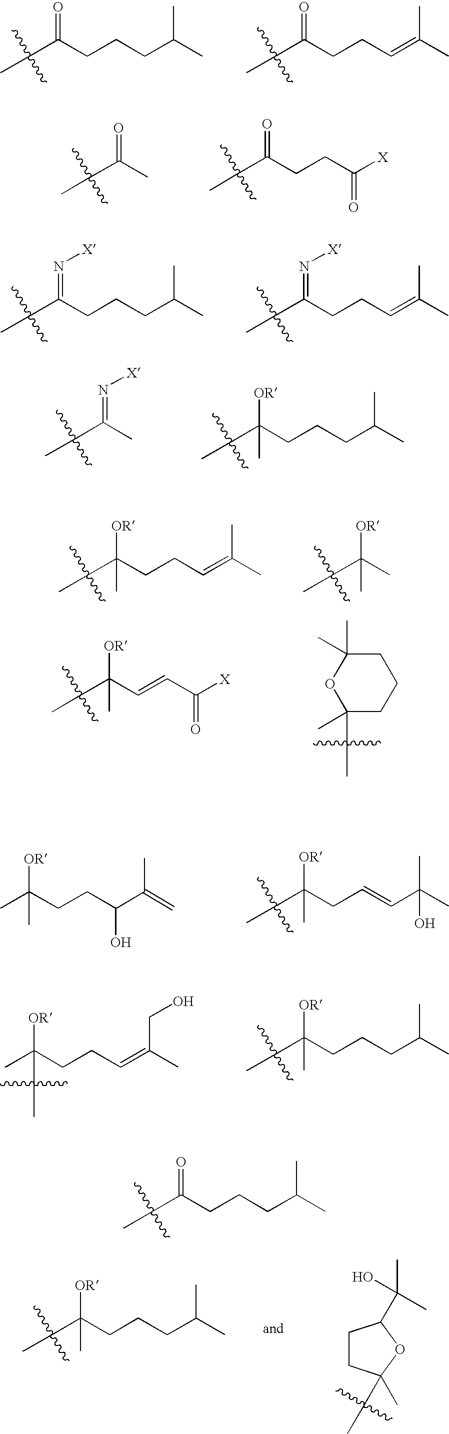
where the configuration of any stereo center is R or S; X is OR or NR, X′ is alkyl, OR or NR; and where R is alkyl or aryl; and R′ is H, alkyl or acyl.

6. The compound of claim 1, where the compound is selected from the group consisting of:

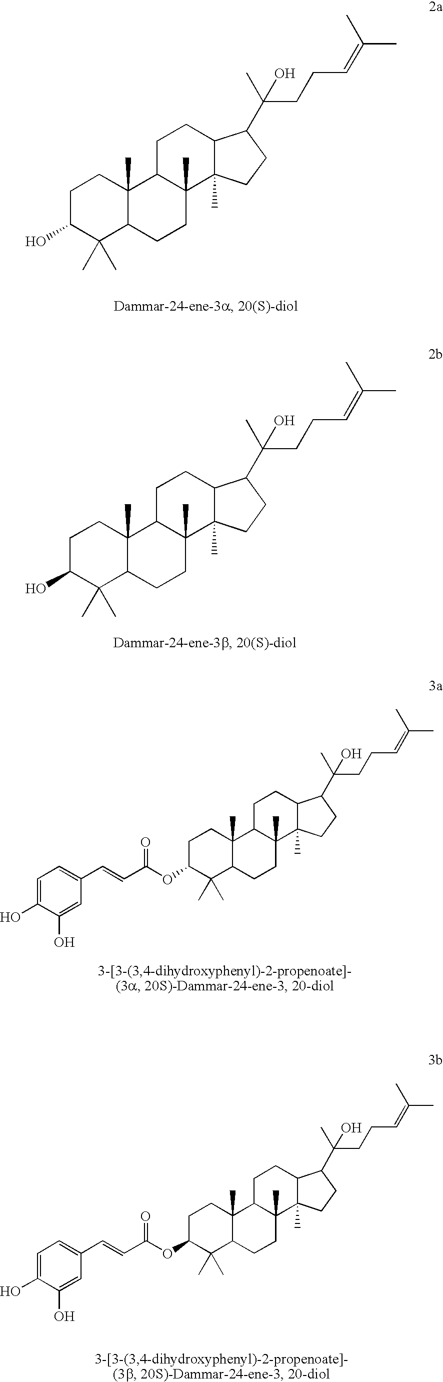

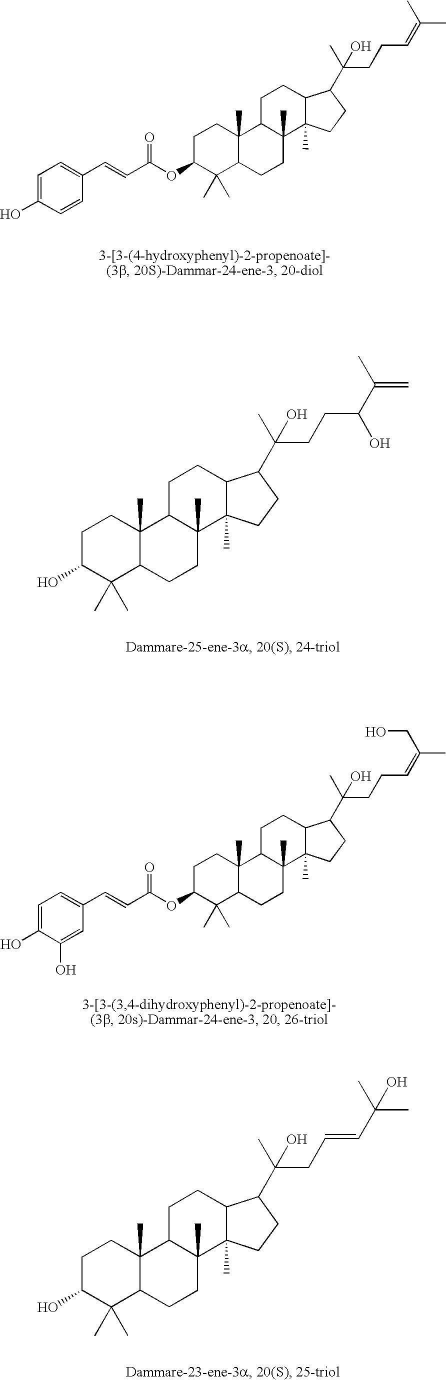

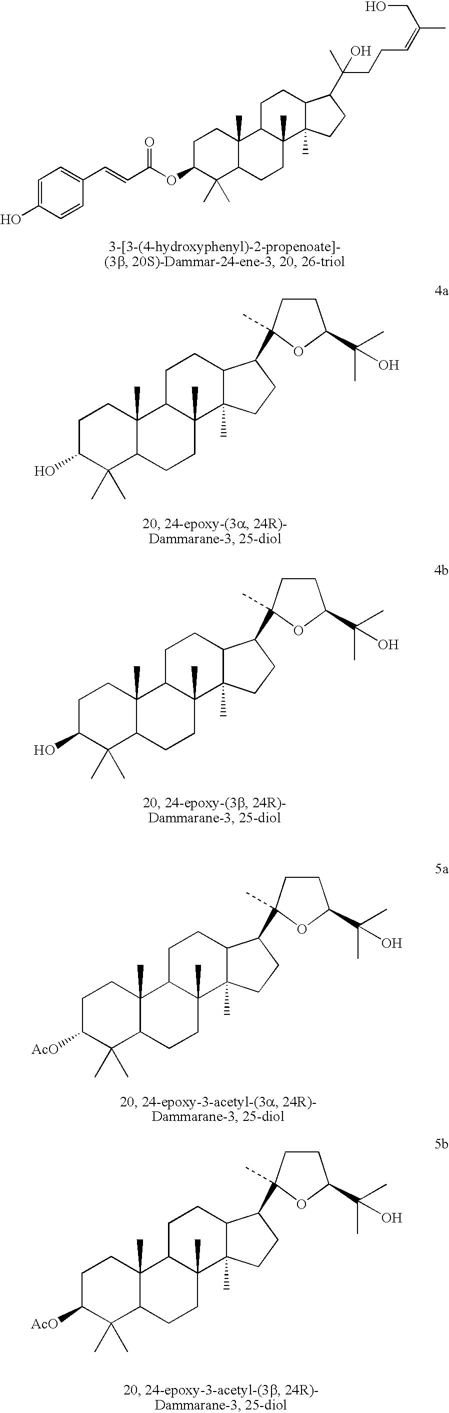

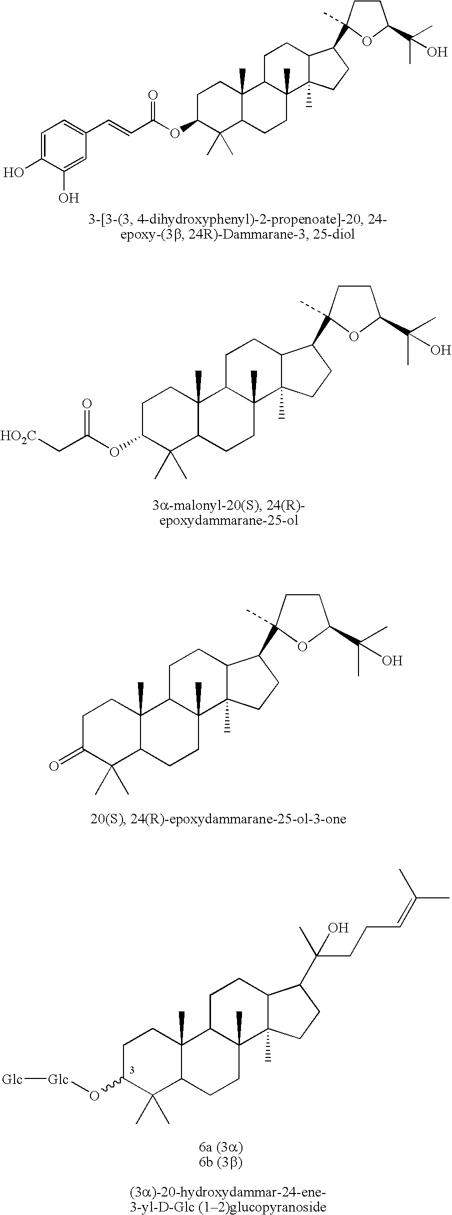

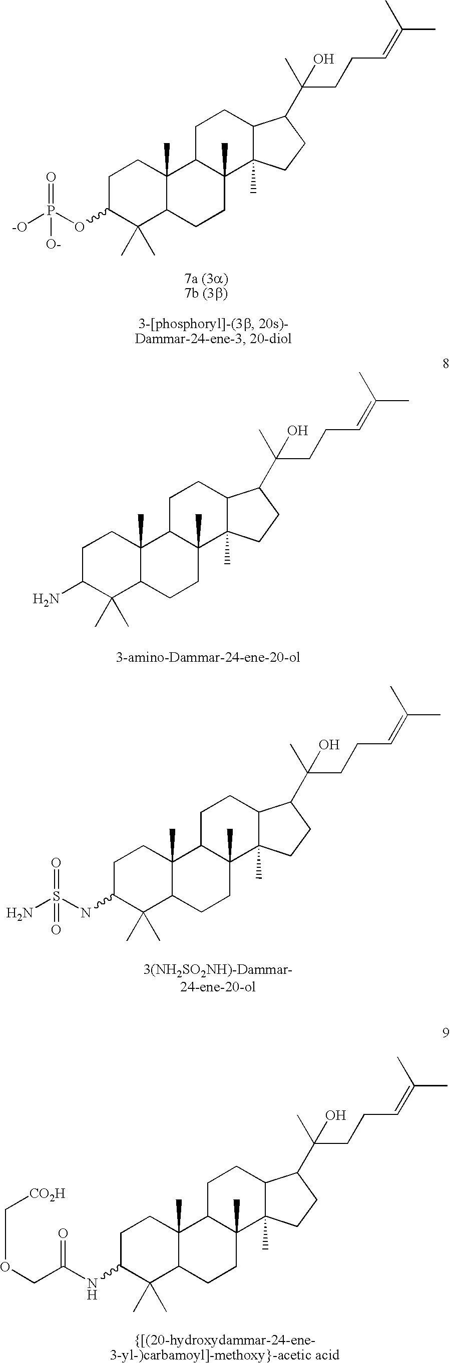

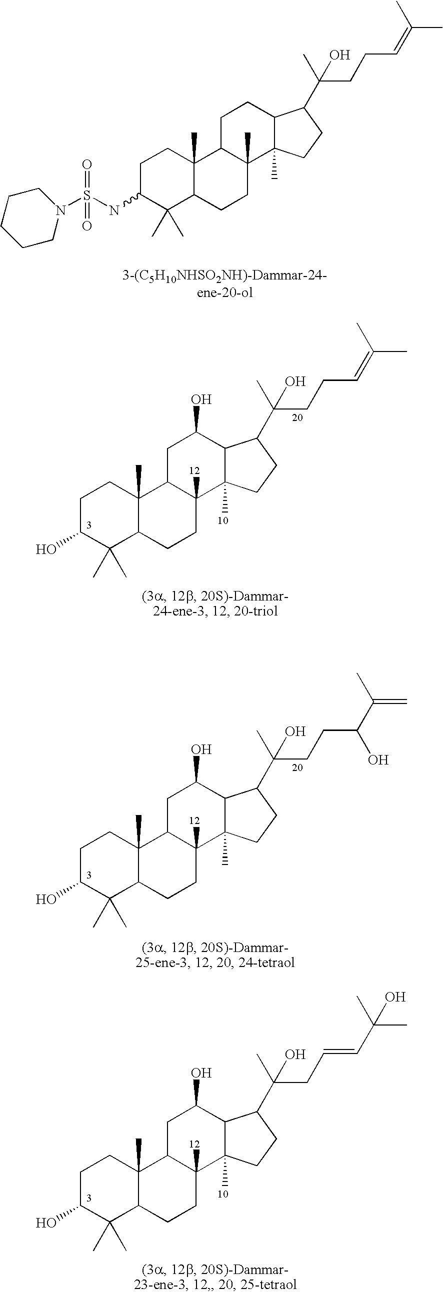

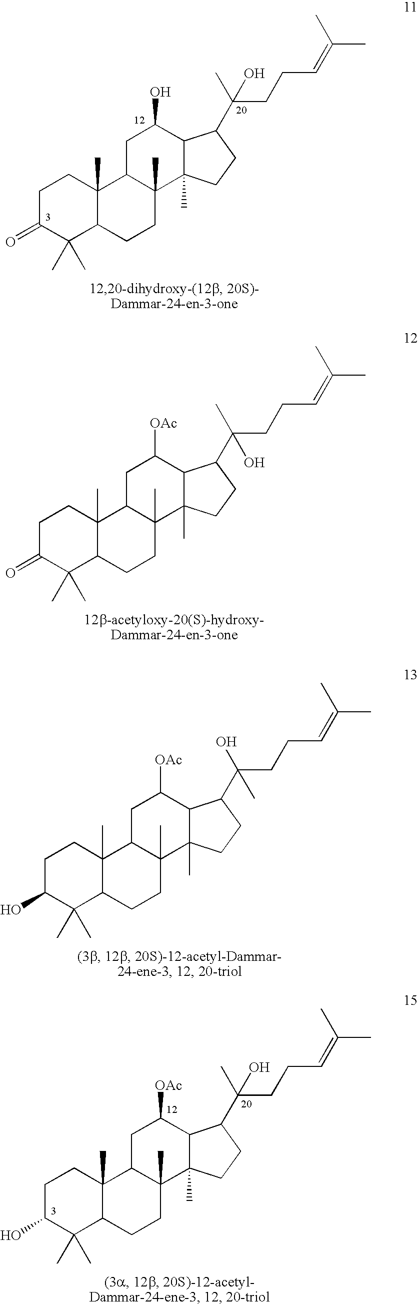

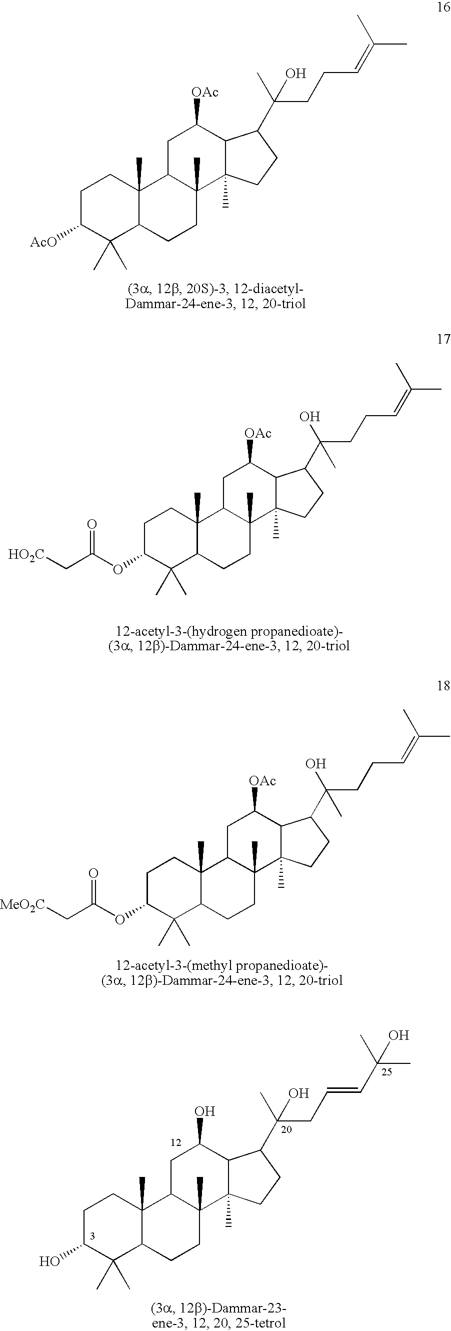

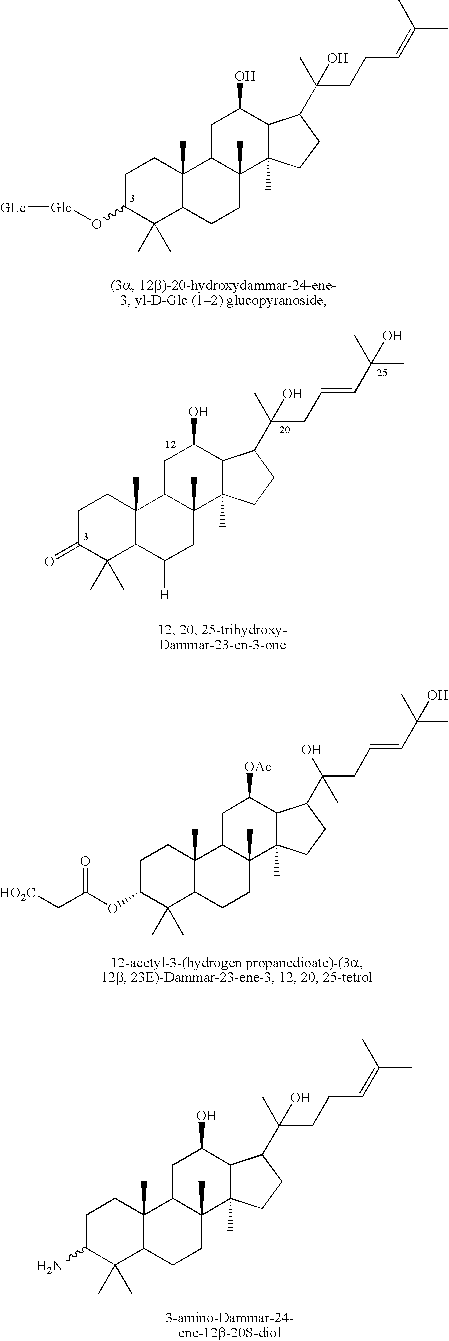

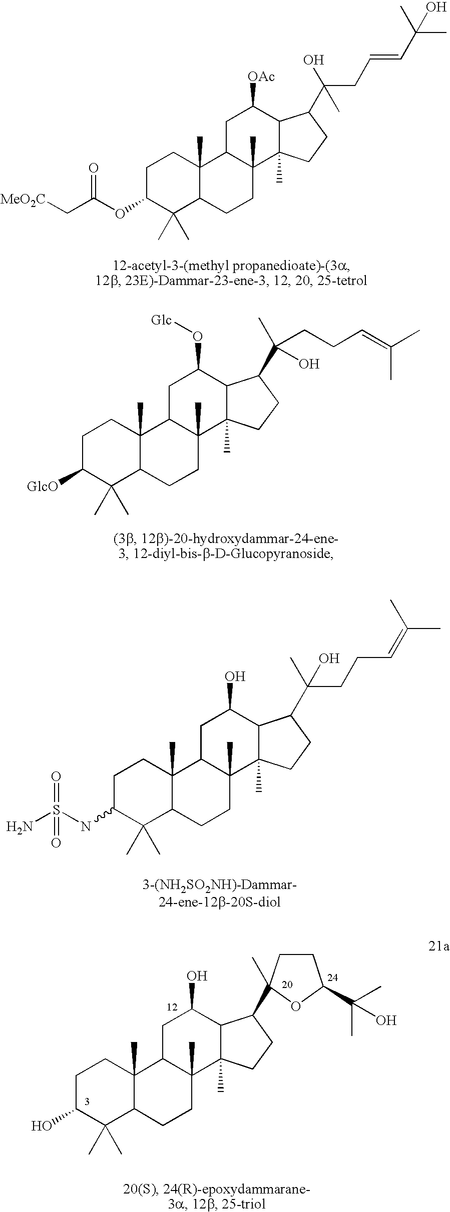

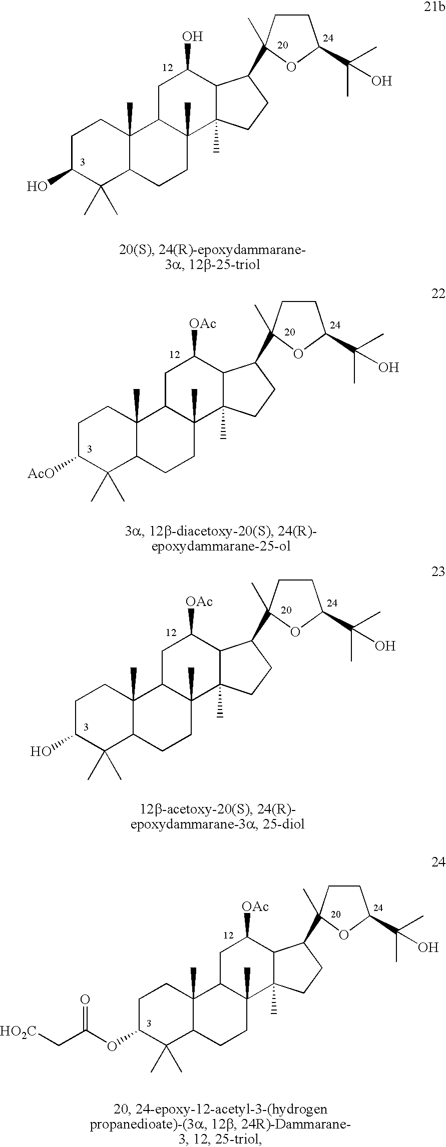

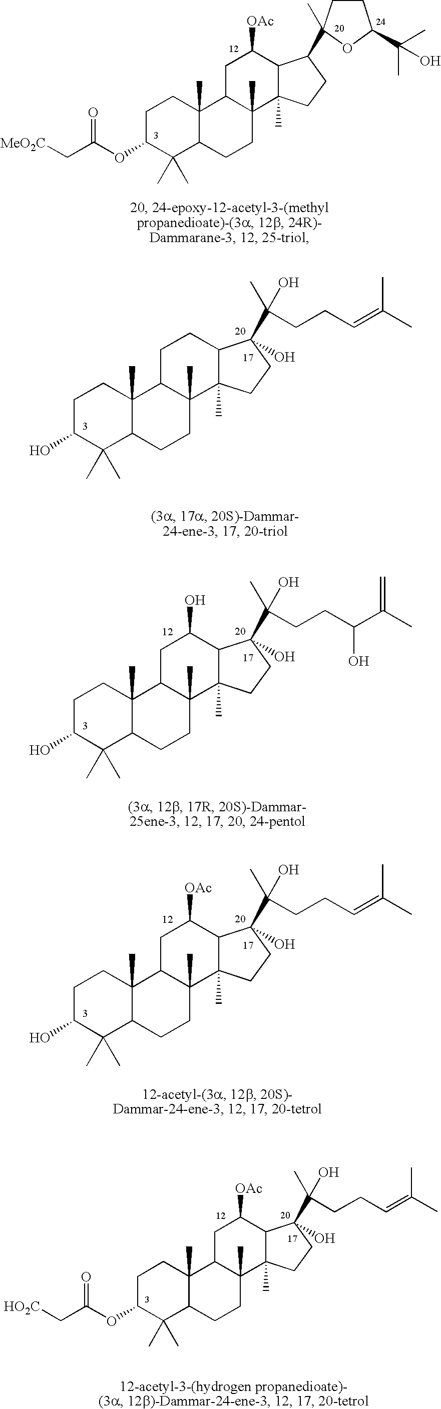

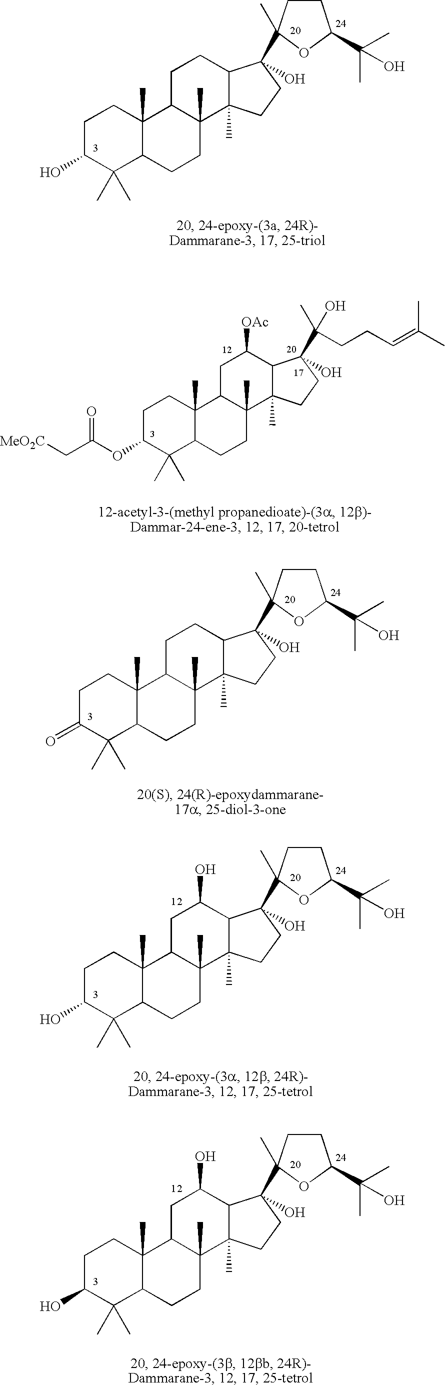

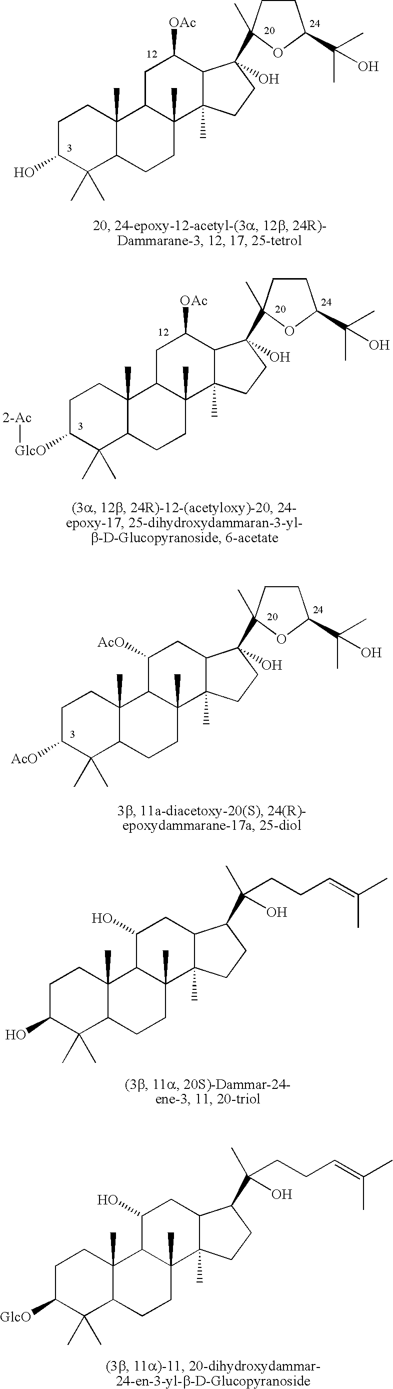

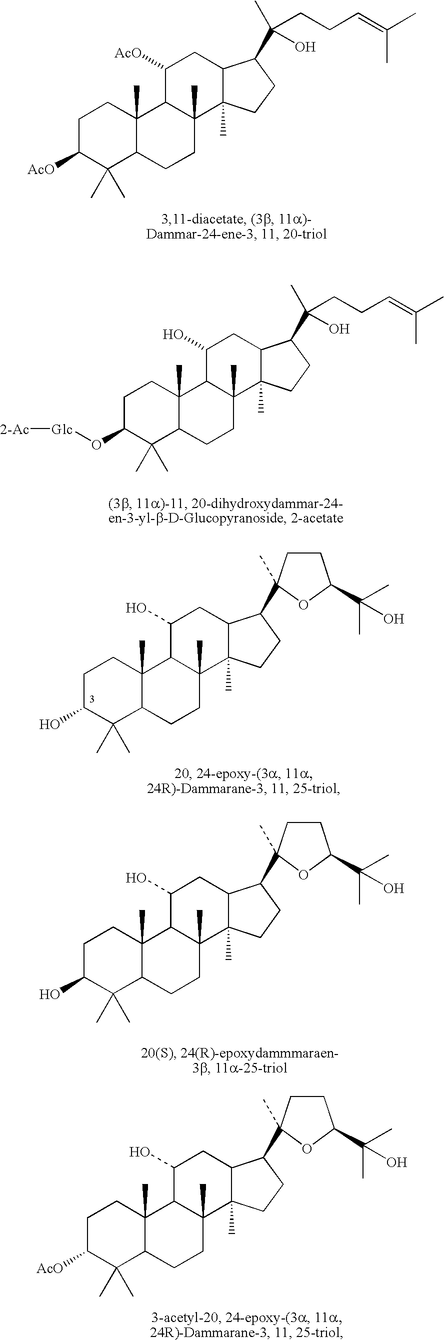

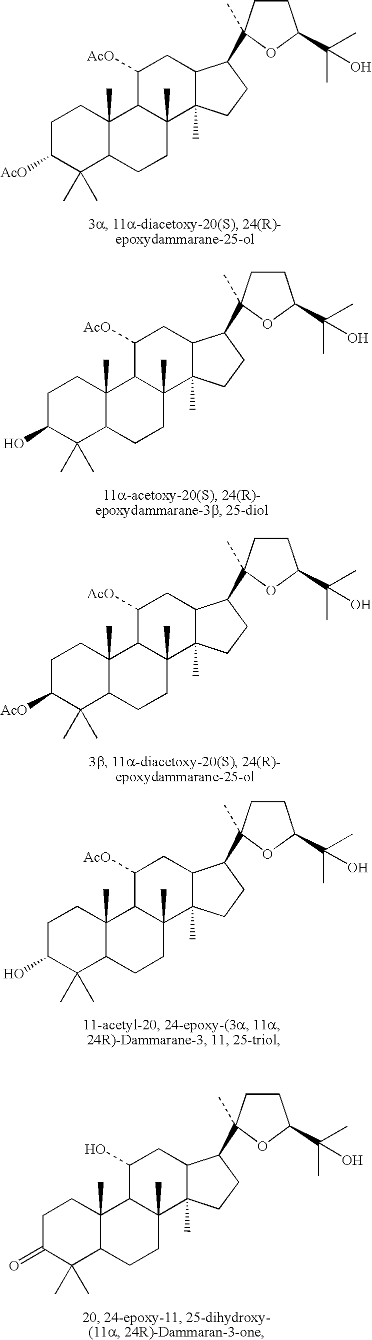

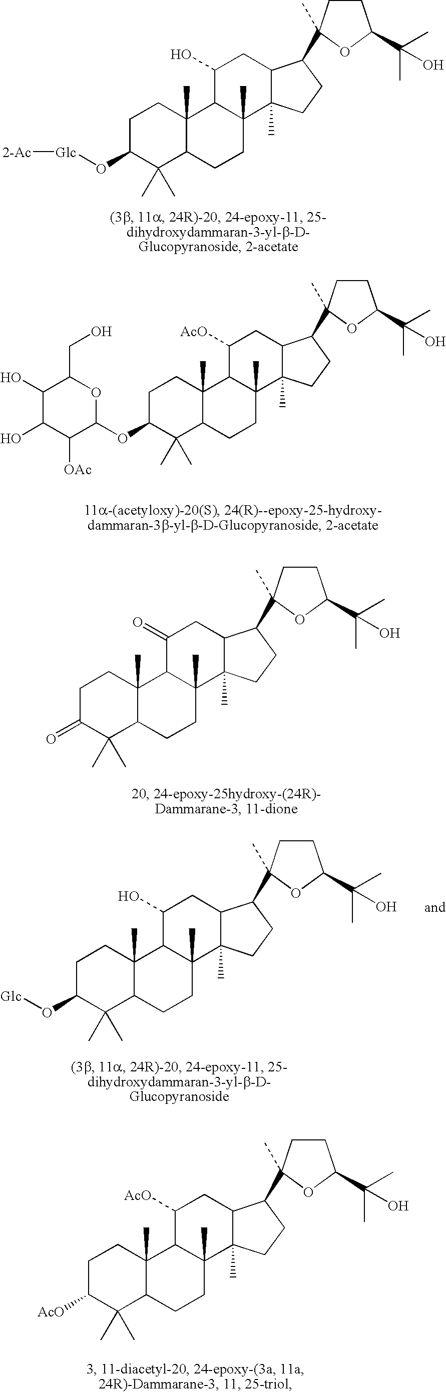

7. Use of a compound having the general formula: 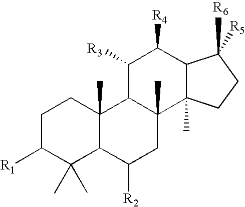
in the treatment or prevention of a pathological condition where R₁ is selected from the group consisting of α-OH, β-OH, α-O—X, β-O—X, α-R₇COO—, β-R₇COO—, α-R₇PO₃—, β-R₇PO₃—, α-NR₈R₉, β-NR₈R₉, ═O(oxo), ═NOH, ═NC(O)NHNH₂, and CH₂—X; where X is an alkyl or a carbohydrate containing one or more sugars or acylated derivatives thereof; R₇ is H, OH, an amino group, an alkenyl, aryl, or alkyl; R₈ is H, alkyl, aryl, acyl, or SO₂NHR₁₀; R₉ is H, alkyl, aryl, acyl, or SO₂NHR₁₀; R₁₀ is NH2, OH, alkyl, aryl, or cycloalkyl;
R₂ is selected from the group consisting of H, OH, OAc, and O—X, where X is a carbohydrate containing one or more sugars or acylated derivatives thereof, Ac═CH₃CO or acyl;
R₃ is selected from the group consisting of H, OH, O—X and OAc, where X is an alkyl or a carbohydrate containing one or more sugars or acylated derivatives thereof;
R₄ is selected from the group consisting of H, OH, O—X and OAc, where X is an alkyl or a carbohydrate containing one or more sugars or acylated derivatives thereof; R5 is H or OH; and
R6 is alkenyl, aryl, or alkyl.

8. The use of claim 6, where R₇ further contains oxygen, nitrogen, or phosphorus.

9. The use of claim 6, where R₆ further contains a function group selected from the group consisting of hydroxyl, ether, ketone, oxime, hydrazone, imine, and Schiff base.

10. The use of claim 6, where the pathological condition is neurodegeneration.

11. The use of claim 6, where the pathological condition is Alzheimer's disease.

12. The use of claim 6, where the pathological condition is an Aβ42-related disorder.

13. An isolated compound having the general formula: 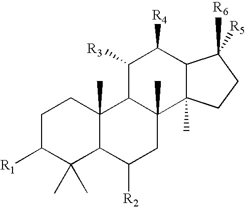
in the treatment or prevention of a pathological condition where R₁ is selected from the group consisting of α-OH, β-OH, α-O—X, β-O—X, α-R₇COO—, β-R₇COO—, α-R₇PO₃—, β-R₇PO₃—, α-NR₈R₉, β-NR₈R₉, ═O(oxo), ═NOH, ═NC(O)NHNH₂, and CH₂—X; where X is an alkyl or a carbohydrate containing one or more sugars or acylated derivatives thereof; R₇ is H, OH, an amino group, an alkenyl, aryl, or alkyl; R₈ is H, alkyl, aryl, acyl, or SO₂NHR₁₀; R₉ is H, alkyl, aryl, acyl, or SO₂NHR₁₀; R₁₀ is NH2, OH, alkyl, aryl, or cycloalkyl;
R₂ is selected from the group consisting of H, OH, OAc, and O—X, where X is a carbohydrate containing one or more sugars or acylated derivatives thereof, Ac═CH₃CO or acyl;
R₃ is selected from the group consisting of H, OH, O—X and OAc, where X is an alkyl or a carbohydrate containing one or more sugars or acylated derivatives thereof;
R₄ is selected from the group consisting of H, OH, O—X and OAc, where X is an alkyl or a carbohydrate containing one or more sugars or acylated derivatives thereof; R5 is H or OH; and
R₆ is alkenyl, aryl, or alkyl.

14. The isolated compound of claim 13, where R₇ further contains oxygen, nitrogen, or phosphorus;

15. The isolated compound of claim 13, where R₆ further contains a functional group selected from the group consisting of hydroxyl, ether, ketone, oxime, hydrazone, imine, and Schiff base.

16. A composition comprising a compound having the general formula: 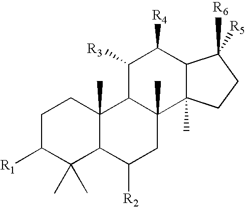
in the treatment or prevention of a pathological condition where R₁ is selected from the group consisting of α-OH, β-OH, α-O—X, β-O—X, α-R₇COO—, β-R₇COO—, α-R₇PO₃—, β-R₇PO₃—, α-NR₈R₉, β-NR₈R₉, ═O(oxo), ═NOH, ═NC(O)NHNH₂, and CH₂—X; where X is an alkyl or a carbohydrate containing one or more sugars or acylated derivatives thereof; R₇ is H, OH, an amino group, an alkenyl, aryl, or alkyl; R₈ is H, alkyl, aryl, acyl, or SO₂NHR₁₀; R₉ is H, alkyl, aryl, acyl, or SO₂NHR₁₀; R₁₀ is NH2, OH, alkyl, aryl, or cycloalkyl;
R₂ is selected from the group consisting of H, OH, OAc, and O—X, where X is a carbohydrate containing one or more sugars or acylated derivatives thereof, Ac═CH₃CO or acyl;
R₃ is selected from the group consisting of H, OH, O—X and OAc, where X is an alkyl or a carbohydrate containing one or more sugars or acylated derivatives thereof;
R₄ is selected from the group consisting of H, OH, O—X and OAc, where X is an alkyl or a carbohydrate containing one or more sugars or acylated derivatives thereof, R5 is H or OH; and
R6 is alkenyl, aryl, or alkyl.

17. The composition of claim 16, where R₇ further contains oxygen, nitrogen, or phosphorus.

18. The composition of claim 16, where R₆ further contains a functional group selected from the group consisting of hydroxyl, ether, ketone, oxime, hydrazone, imine, and Schiff base.

19. A pharmaceutical composition comprising a pharmaceutically acceptable carrier and a compound having the general formula: 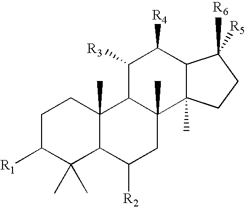
where R₁ is selected from the group consisting of α-OH, β-OH, α-O—X, β-O—X, α-R₇COO—, β-R₇COO—, α-R₇PO₃—, β-R₇PO₃—, α-NR₈R₉, β-NR₈R₉, ═O(oxo), ═NOH, ═NC(O)NHNH₂, and CH₂—X; where X is an alkyl or a carbohydrate containing one or more sugars or acylated derivatives thereof; R₇ is H, OH, an amino group, an alkenyl, aryl, or alky; R₈ is H, alkyl, aryl, acyl, or SO₂NHR₁₀; R₉ is H, alkyl, aryl, acyl, or SO₂NHR₁₀; R₁₀ is NH2, OH, alkyl, aryl, or cycloalkyl;
R₂ is selected from the group consisting of H, OH, OAc, and O—X, where X is a carbohydrate containing one or more sugars or acylated derivatives thereof, Ac═CH₃CO or acyl;
R₃ is selected from the group consisting of H, OH, O—X and OAc, where X is an alkyl or a carbohydrate containing one or more sugars or acylated derivatives thereof,
R₄ is selected from the group consisting of H, OH, O—X and OAc, where X is an alkyl or a carbohydrate containing one or more sugars or acylated derivatives thereof, R5 is H or OH; and
R6 is alkenyl, aryl, or alkyl.

20. The pharmaceutical composition of claim 98, where R₇ further contains oxygen, nitrogen, or phosphorus.

21. The pharmaceutical composition of claim 19, where R₆ further contains a function group selected from the group consisting of hydroxyl, ether, ketone, oxime, hydrazone, imine, and Schiff base.

22. A method of synthesis of a compound having the general formula: 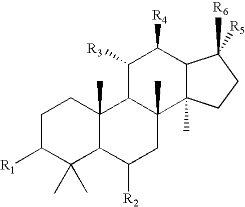
the method comprising the steps of:
(a) treating a compound having formula: 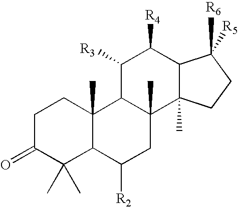
with a reducing agent, followed by an appropriate oxidizing agent to form a compound having formula: 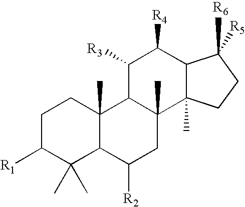
where R₁ is selected from the group consisting of α-OH, β-OH, α-O—X, β-O—X, α-R₇COO—, β-R₇COO—, α-R₇PO₃—, β-R₇PO₃—, α-NR₈R₉, β-NR₈R₉, ═O(oxo), ═NOH, ═NC(O)NHNH₂, and CH₂—X; where X is an alkyl or a carbohydrate containing one or more sugars or acylated derivatives thereof; R₇ is H, OH, an amino group, an alkenyl, aryl, or alkyl; R₈ is H, alkyl, aryl, acyl, or SO₂NHR₁₀; R₉ is H, alkyl, aryl, acyl, or SO₂NHR₁₀; R₁₀ is NH2, OH, alkyl, aryl, or cycloalkyl;
R₂ is selected from the group consisting of H, OH, OAc, and O—X, where X is a carbohydrate containing one or more sugars or acylated derivatives thereof, Ac═CH₃CO or acyl;
R₃ is selected from the group consisting of H, OH, O—X and OAc, where X is an alkyl or a carbohydrate containing one or more sugars or acylated derivatives thereof;
R₄ is selected from the group consisting of H, OH, O—X and OAc, where X is an alkyl or a carbohydrate containing one or more sugars or acylated derivatives thereof; R5 is H or OH; and
R6 is alkenyl, aryl, or alkyl.

23. The method of claim 22, where the compound having the formula: 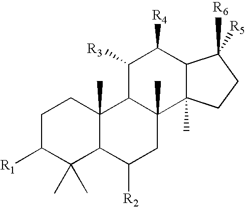
is dipterocarpol from dammar resin.

24. The method of claim 22, where the compound having formula: 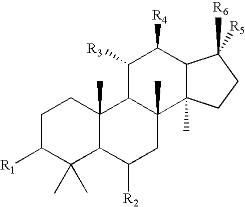
is obtained from a plant.

25. The method of claim 22, where the plant is selected from the group consisting of birch.

26. A method for the synthesis of a compound having the formula: 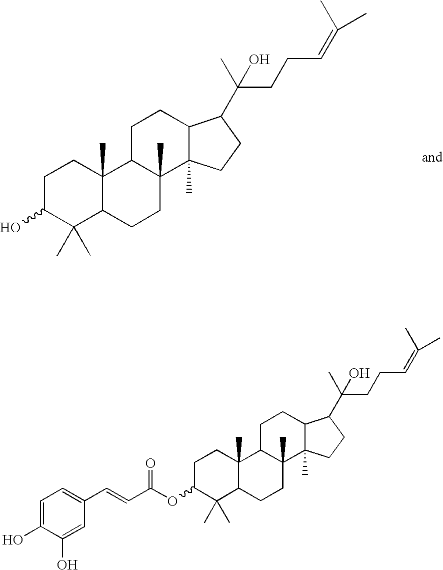
the method comprising the steps of:
(a) treating a compound having formula: 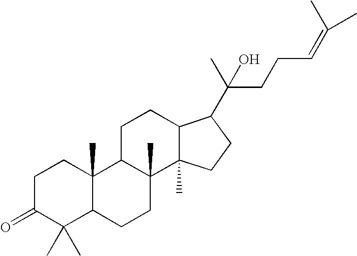
with a reducing agent, to form a compound having formula: 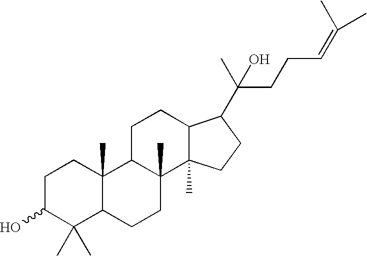
(b) treating the compound formed in step (a) with an esterificating agent followed by a hydrolyzing agent, to form a compound having formula:

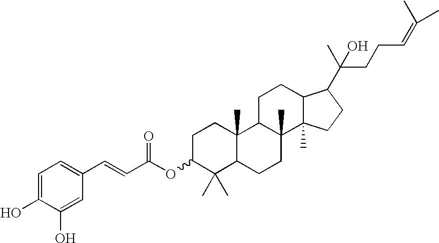

27. The method of claim 26, where the reducing agent is NaBH₄.

28. The method of claim 26, where the esterificating agent is di-O-acetylcaffeoyl chloride and the hydrolyzing agent is NaHCO₃.

29. A method for the synthesis of a compound having the formula: 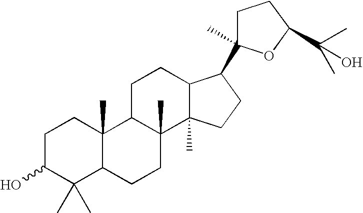
the method comprising the steps of:
(a) treating a compound having formula: 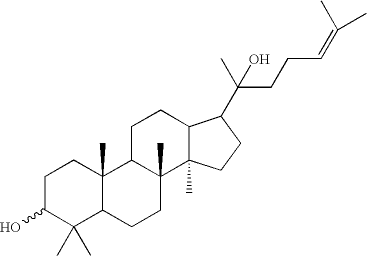
with an oxidizing agent, to form a compound having formula: 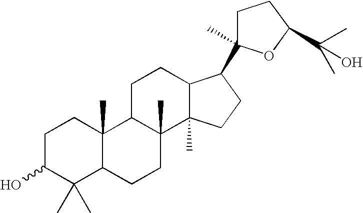
(b) treating the compound formed in step (a) with an esterificating agent to form a compound having formula:

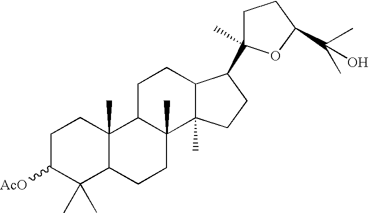

30. The method of claim 29, where the oxidizing agent is MCPBA.

31. The method of claim 29, where the esterificating agent is Ac₂O.

32. A method for the synthesis of a compound having the formula: 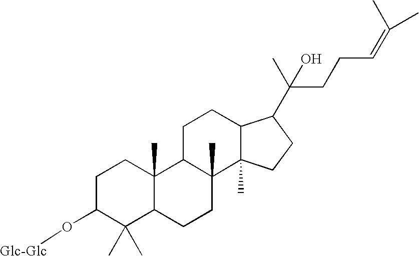
the method comprising the steps of:
(a) treating a compound having formula: 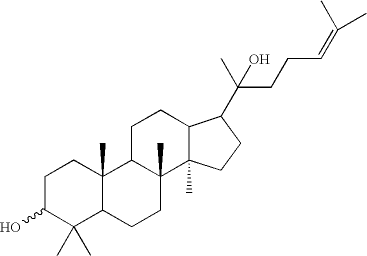
with an appropriate bromonated sugar and an oxidizing agent, followed by deprotection of the acetyl groups to form a compound having formula:

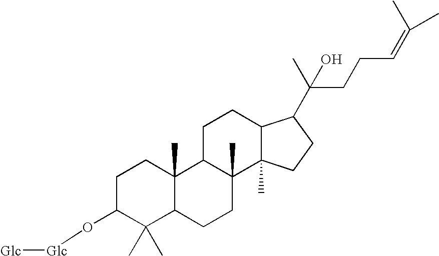

33. The method of claim 32, where the oxidizing agent is Ag2O.

34. The method of claim 32, where the compound is deprotected using NaOMe.

35. A method for the synthesis of a compound having the formula: 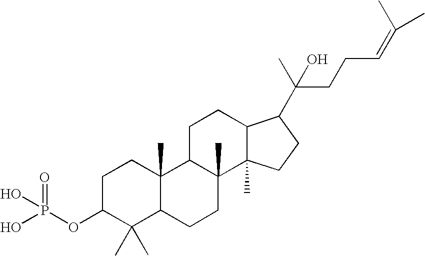
The method comprising the steps of:
(a) treating a compound having formula: 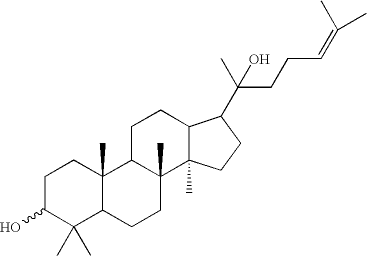
with a dibenzylphosphoric agent and a base to form a compound having formula: 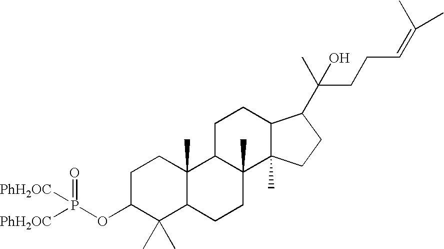
(b) treating the compound formed in step (a) with a hydrogenating agent to form a compound having formula:

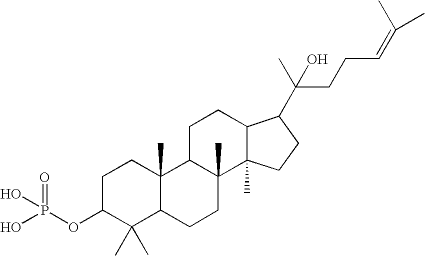

36. The method of claim 35, where the dibenzylphosphoric agent is (PhCH₂O)₂POCl.

37. The method of claim 35, where the hydrogenating agent is H₂/Pd—C.

38. A method for the synthesis of a compound having the general formula: 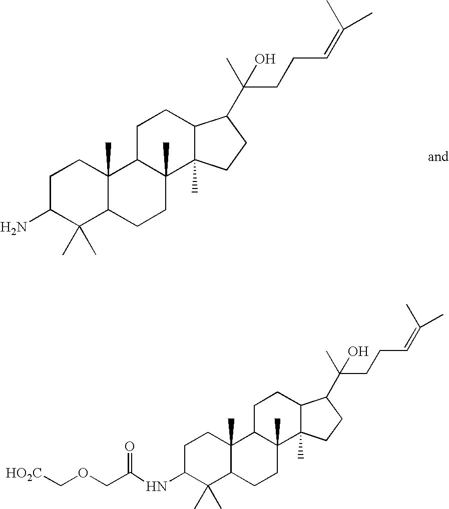
the method comprising the steps of:
(a) treating a compound having formula: 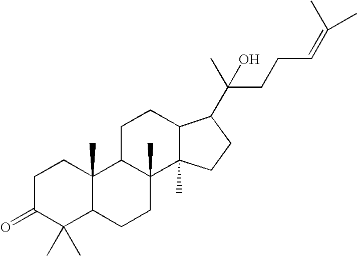
with an reducing agent to form a compound having formula: 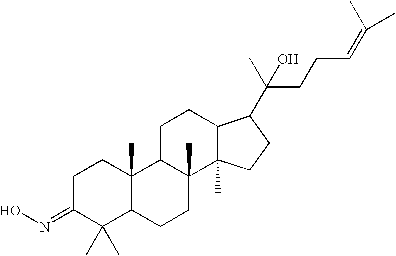
(b) treating the compound formed in step (a) with an additional reducing agent to form a compound having formula: 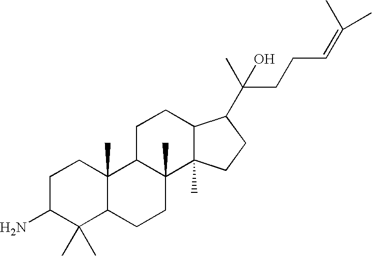
(c) treating the compound formed in step (b) with diglylcolic anhydride in the presence of a base to form a compound having formula:

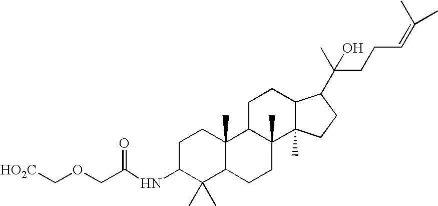

39. The method of claim 38, where the reducing agent of step (a) is NH2OH.

40. The method of claim 38, where the reducing agent of step (b) is Na.

41. A method for the synthesis of a compound having the formula: 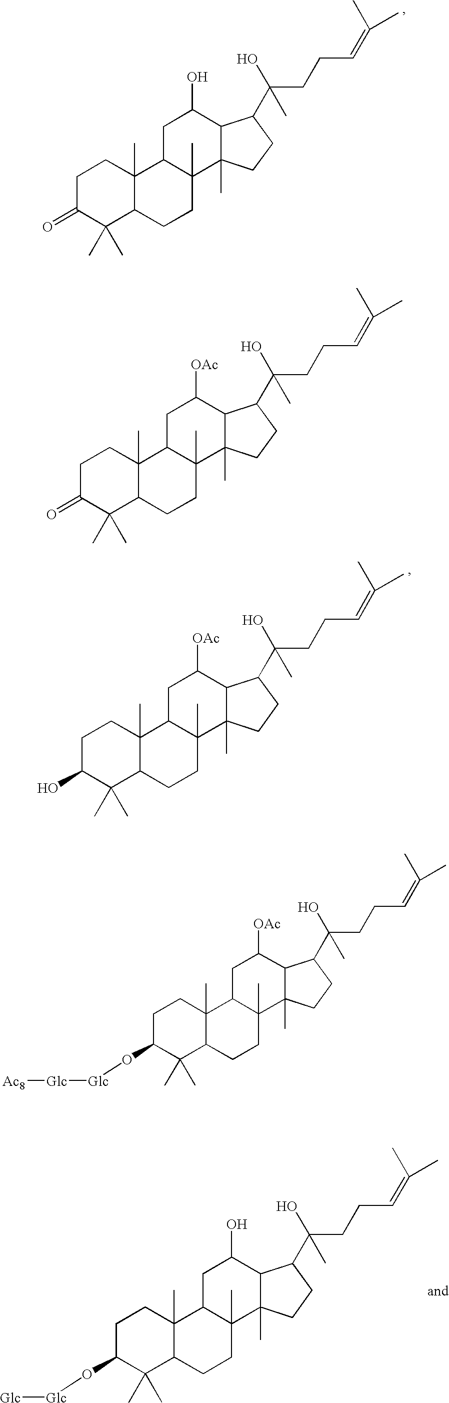 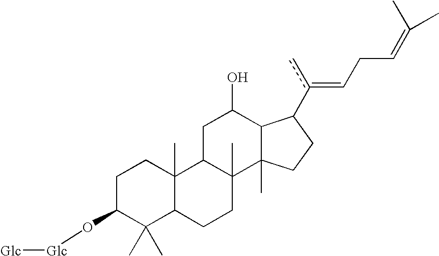
the method comprising the steps of:
(a) treating a compound having formula: 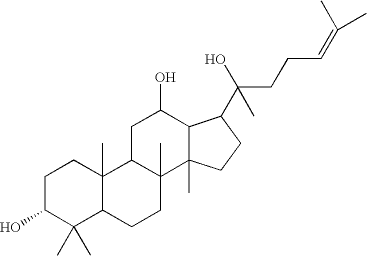
with an oxidizing agent to form a compound having formula: 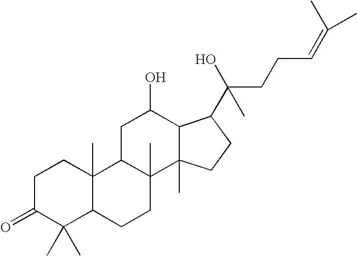
(b) treating the compound formed in step (a) with an acetylating agent to form a compound having formula: 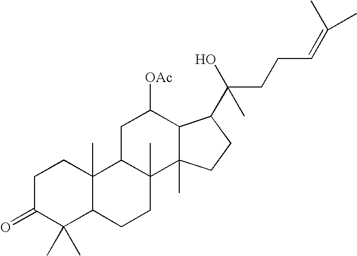
(c) treating the compound formed in step (b) with a reducing agent to form a compound having formula: 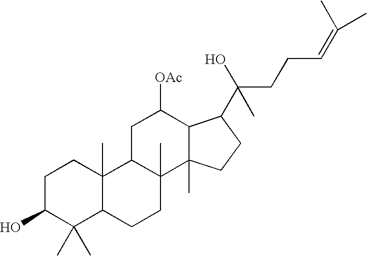
(d) treating the compound formed in step (c) with Ac₈-Glc-Glc-Br in the presence of Ag₂O to form a compound having formula: 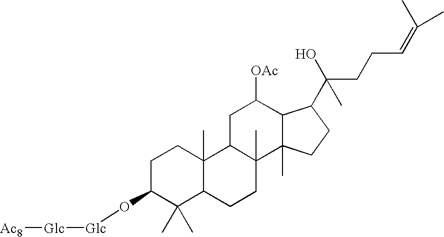
(e) treating the compound formed in step (d) with a deacetylating agent to form a compound having formula: 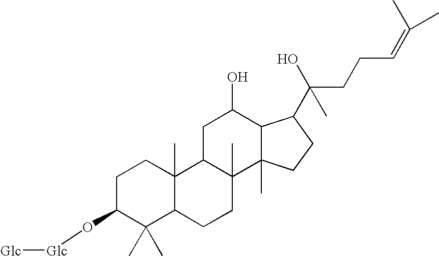
(f) treating the compound formed in step (e) with a dehydrating agent to form a compound having formula:

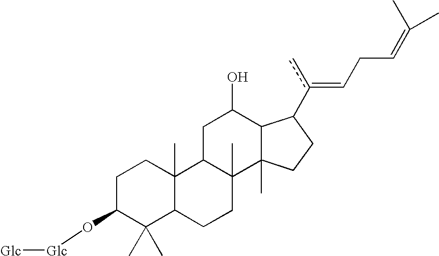

42. The method of claim 41 where the compound of formula: 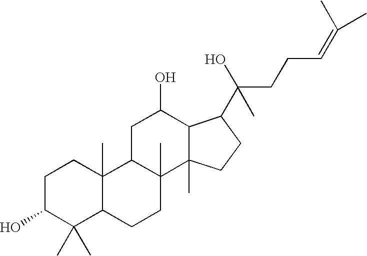
is obtained from a plant.

43. The method of claim 41, where the plant is selected from the group consisting of birch.

44. The method of claim 41, where the oxidizing agent is CrO₃ in pyridine.

45. The method of claim 41, where the acetylating agent is Ac₂O in pyridine.

46. The method of claim 41, where the reducing agent is NaBH₄.

47. The method of claim 41, where the deacetylating agent is NaOMe, and the dehydrating agent is mesyl chloride and triethylamine.

48. A method for the synthesis of a compound having the formula: 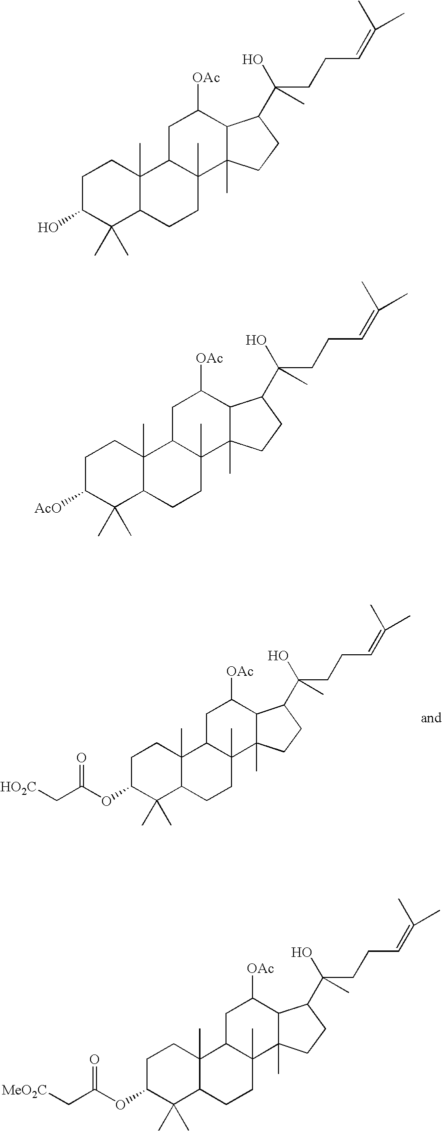
the method comprising the steps of:
(a) treating a compound having formula: 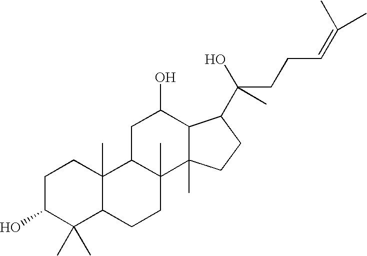
with Ac₂O to form a compound having formula: 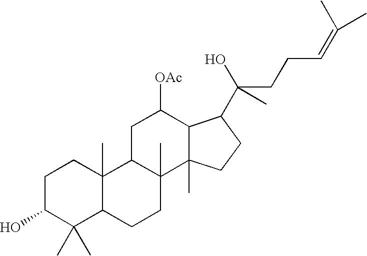
(b) treating the compound formed in step (a) with Ac₂O to form a compound having formula: 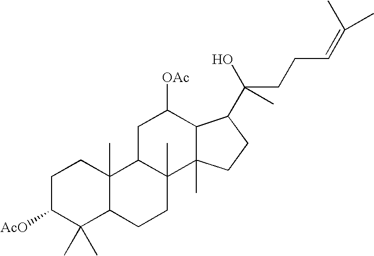
(c) treating the compound formed in step (a) with CH₂(COCl)₂ to form a compound having formula: 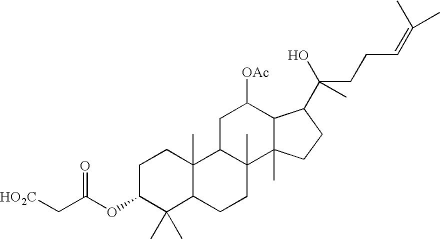
(d) treating the compound formed in step (c) with CH₂N₂ to form a compound having formula:

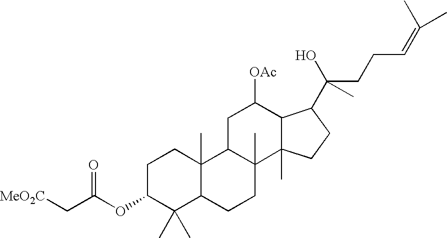

49. The method of claim 48, where the compound of formula: 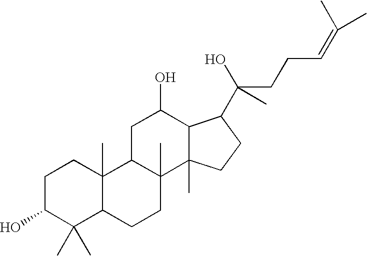
is obtained from a plant.

50. The method of claim 48, where the plant is selected from the group consisting of birch.

51. A method for the synthesis of a compound having the formula: 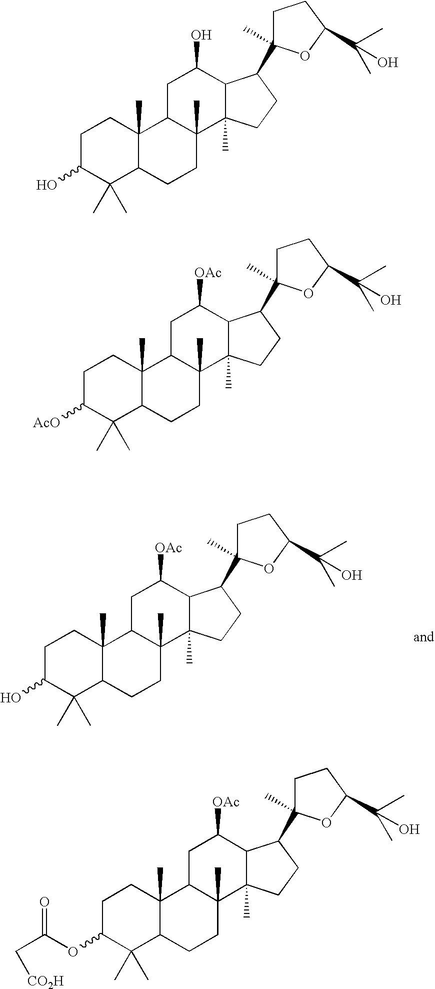
the method comprising the steps of:
(a) treating a compound having formula: 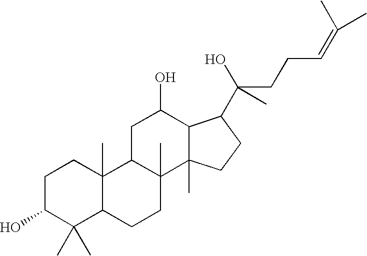
with an oxidizing agent to form a compound having formula: 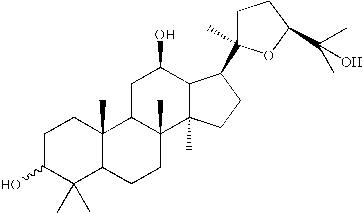
(b) treating the compound formed in step (a) with an acid anhydride or acid chloride to form a compound having formula:

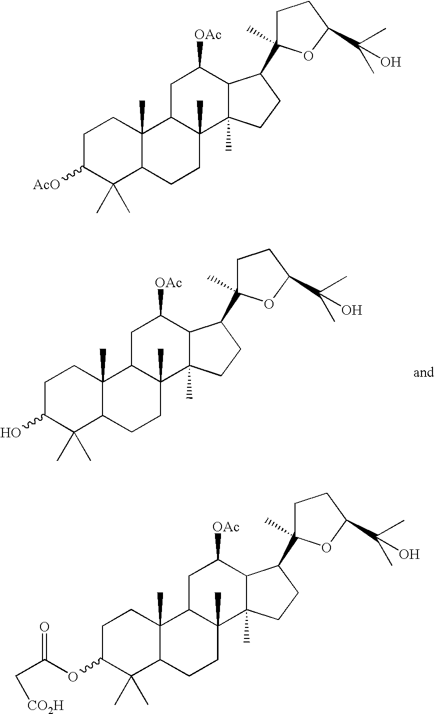

52. The method of claim 51, where the compound of formula: 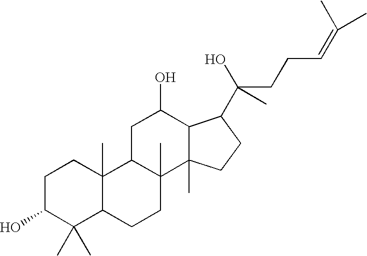
is obtained from a plant.

53. The method of claim 51, where the plant is selected from the group consisting of birch.

54. The method of claim 51, where the oxidizing agent is MCPBA.

55. The method of claim 51, where the acid anhydride is (RCO)₂O.

56. A method for treating or preventing a pathological condition in a subject, comprising administering a compound having the general formula: 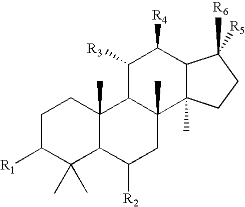
where R₁ is selected from the group consisting of α-OH, β-OH, α-O—X, β-O—X, α-R₇COO—, β-R₇COO—, α-R₇PO₃—, β-R₇PO₃—, α-NR₈R₉, β-NR₈R₉, ═O(oxo), ═NOH, ═NC(O)NHNH₂, CH₂—X; where X is an alkyl or a carbohydrate containing one or more sugars or acylated derivatives thereof; R₇ is H, OH, an amino group, an alkenyl, aryl, or alkyl; R₈ is H, alkyl, aryl, acyl, or SO₂NHR₁₀; R₉ is H, alkyl, aryl, acyl, or SO₂NHR₁₀; R₁₀ is NH2, OH, alkyl, aryl, or cycloalkyl;
R₂ is selected from the group consisting of H, OH, OAc, and O—X, where X is a carbohydrate containing one or more sugars or acylated derivatives thereof, Ac═CH₃CO or acyl;
R₃ is selected from the group consisting of H, OH, O—X and OAc, where X is an alkyl or a carbohydrate containing one or more sugars or acylated derivatives thereof;
R₄ is selected from the group consisting of H, OH, O—X and OAc, where X is an alkyl or a carbohydrate containing one or more sugars or acylated derivatives thereof; R5 is H or OH; and
R6 is alkenyl, aryl, or alkyl.

57. The method of claim 56, where R₇ further contains oxygen, nitrogen, or phosphorus; and R₆ further contains a function group selected from the group consisting of hydroxyl, ether, ketone, oxime, hydrazone, imine, and Schiff base.

58. The method of claim 56, where the pathological condition is neurodegeneration.

59. The method of claim 56, where the pathological condition is Alzheimer's disease.

60. The method of claim 56, where the pathological condition is an Aβ42-related disorder.

61. The method of claim 56, where the subject is a human.

62. A method for inhibiting O-amyloid production in a subject, comprising administering a compound having the general formula: 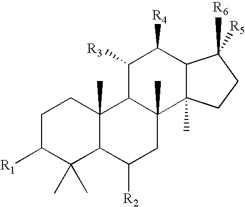
where R₁ is selected from the group consisting of α-OH, β-OH, α-O—X, β-O—X, αR-₇COO—, β-R₇COO—, α-R₇PO₃—, β-R₇PO₃—, α-NR₈R₉, β-NR₈R₉, ═O(oxo), ═NOH, ═NC(O)NHNH₂, CH₂—X; where X is an alkyl or a carbohydrate containing one or more sugars or acylated derivatives thereof; R₇ is H, OH, an amino group, an alkenyl, aryl, or alkyl; R₈ is H, alkyl, aryl, acyl, or SO₂NHR₁₀; R₉ is H, alkyl, aryl, acyl, or SO₂NHR₁₀; R₁₀ is NH2, OH, alkyl, aryl, or cycloalkyl;
R₂ is selected from the group consisting of H, OH, OAc, and O—X, where X is a carbohydrate containing one or more sugars or acylated derivatives thereof, Ac═CH₃CO or acyl;
R₃ is selected from the group consisting of H, OH, O—X and OAc, where X is an alkyl or a carbohydrate containing one or more sugars or acylated derivatives thereof;
R₄ is selected from the group consisting of H, OH, O—X and OAc, where X is an alkyl or a carbohydrate containing one or more sugars or acylated derivatives thereof; R5 is H or OH; and
R6 is alkenyl, aryl, or alkyl.

63. The method of claim 62, where the R₇ further contains oxygen, nitrogen, or phosphorus; and R₆ further contains a function group selected from the group consisting of hydroxyl, ether, ketone, oxime, hydrazone, imine, and Schiff base.
